SEQUENCE LISTING (124 sequences)

SEQ ID NO: 1 - protein, 17 aa, Felis catus
LFLTGTPDEYVEQVAQY

SEQ ID NO: 2 - protein, 16 aa, Felis catus
EQVAQYKALPVVLENA

SEQ ID NO: 3 - protein, 17 aa, Felis catus
KALPVVLENARILKNCV

SEQ ID NO: 4 - protein, 70 aa, Felis catus
EICPAVKDRVDLFLTGTPDEYVEQVAQYKALPVVLENARILKNCVDAKMTEEDKENALSLLDKIYTSPLC

SEQ ID NO: 5 - protein, 92 aa, Felis catus
VKMAETCPIFYDVFFAVANGNELLLKLSLTKVNATEPERTAMKKIQDCYVENGLISRVLDGLVMTTISSSKDCMGEAVQNTVEDLKLNTLGR

SEQ ID NO: 6 - protein, 17 aa, Felis catus
EICPAVKRDVDLFLTGT

SEQ ID NO: 7 - protein, 17 aa, Felis catus
RILKNCVDAKMTEEDKE

SEQ ID NO: 8 - protein, 16 aa, Felis catus
KMTEEDKENALSLLDK

SEQ ID NO: 9 - protein, 16 aa, Felis catus
KENALSVLDKIYTSPL

SEQ ID NO: 10 - protein, 16 aa, Felis catus
VKMAETCPIFYDVFFA

SEQ ID NO: 11 - protein, 17 aa, Felis catus
CPIFYDVFFAVANGNEL

SEQ ID NO: 12 - protein, 16 aa, Felis catus
GNELLLKLSLTKVNAT

SEQ ID NO: 13 - protein, 16 aa, Felis catus
LTKVNATEPERTAMKK

SEQ ID NO: 14 - protein, 16 aa, Felis catus
TAMKKIQDCYVENGLI

SEQ ID NO: 15 - protein, 16 aa, Felis catus
CYVENGLISRVLDGLV

SEQ ID NO: 16 - protein, 16 aa, Felis catus
SRVLDGLVMTTISSSK

SEQ ID NO: 17 - protein, 16 aa, Felis catus
ISSSKDCMGEAVQNTV

SEQ ID NO: 18 - protein, 16 aa, Felis catus
AVQNTVEDLKLNTLGR

SEQ ID NO: 19 - protein, 320 aa, Dermatophagoides pteronyssinus
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 20 - protein, 146 aa, Dermatophagoides pteronyssinus
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 21 - protein, 261 aa, Dermatophagoides pteronyssinus
MIIYNILIVLLLAINTLANPILPASPNATIVGGEKALAGECPYQISLQSSSHFCGGTILDEYWILTAAHCVAGQTASKLSIRYNSLKHSLGGEKISVAKIFAHEKYDSYQIDNDIALIKLKSPMKLNQKNAKAVGLPAKGSDVKVGDQVRVSGWGYLEEGSYSLPSELRRVDIAVVSRKECNELYSKANAEVTDNMICGGDVANGGKDSCQGDSGGPVVDVKNNQVVGIVSWGYGCARKGYPGVYTRVGNFIDWIESKRSQ

SEQ ID NO: 22 - protein, 19 aa, Dermatophagoides pteronyssinus
  misc_feature: X is an unknown amino acid
KYXNPHFIGXRSVITXLME

SEQ ID NO: 23 - protein, 132 aa, Dermatophagoides pteronyssinus
MKFIIAFFVATLAVMTVSGEDKKHDYQNEFDFLLMERIHEQIKKGELALFYLQEQINHFEEKPTKEMKDKIVAEMDTIIAMIDGVRGVLDRLMQRKDLDIFEQYNLEMAKKSGDILERDLKKEEARVKKIEV

SEQ ID NO: 24 - protein, 20 aa, Dermatophagoides pteronyssinus
  misc_feature: X ia unknown amino acid
AIGXQPAAEAEAPFQISLMK

SEQ ID NO: 25 - protein, 215 aa, Dermatophagoides pteronyssinus
MMKLLLIAAAAFVAVSADPIHYDKITEEINKAVDEAVAAIEKSETFDPMKVPDHSDKFERHIGIIDLKGELDMRNIQVRGLKQMKRVGDANVKSEDGVVKAHLLVGVHDDVVSMEYDLAYKLGDLHPNTHVISDIQDFVVELSLEVSEEGNMTLTSFEVRQFANVVNHIGGLSILDPIFAVLSDVLTAIFQDTVRAEMTKVLAPAFKKELERNNQ

SEQ ID NO: 26 - protein, 18 aa, Dermatophagoides pteronyssinus
IVGGSNASPGDAVYQIAL

SEQ ID NO: 27 - protein, 319 aa, Dermatophagoides farinae
MKFVLAIASLLVLTVYARPASIKTFEFKKAFNKNYATVEEEEVARKNFLESLKYVEANKGAINHLSDLSLDEFKNRYLMSAEAFEQLKTQFDLNAETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQRCRRPNSQHYGISNYCQIYPPDVKQIREALTQTHTAIAVIIGIKDLRAFQHYDGRTIIQHDNGYQPNYHAVNIVGYGSTQGDDYWIVRNSWDTTWGDSGYGYFQAGNNLMMIEQYPYVVIM

SEQ ID NO: 28 - protein, 146 aa, Dermatophagoides farinae
MISKILCLSLLVAAVVADQVDVKDCANNEIKKVMVDGCHGSDPCIIHRGKPFTLEALFDANQNTKTAKIEIKASLDGLEIDVPGIDTNACHFMKCPLVKGQQYDIKYTWNVPKIAPKSENVVVTVKLIGDNGVLACAIATHGKIRD

SEQ ID NO: 29 - protein, 259 aa, Dermatophagoides farinae
MMILTIVVLLAANILATPILPSSPNATIVGGVKAQAGDCPYQISLQSSSHFCGGSILDEYWILTAAHCVNGQSAKKLSIRYNTLKHASGGEKIQVAEIYQHENYDSMTIDNDVALIKLKTPMTLDQTNAKPVPLPAQGSDVKVGDKIRVSGWGYLQEGSYSLPSELQRVDIDVVSREQCDQLYSKAGADVSENMICGGDVANGGVDSCQGDSGGPVVDVATKQIVGIVSWGYGCARKGYPGVYTRVGNFVDWIESKRSQ

SEQ ID NO: 30 - protein, 20 aa, Dermatophagoides farinae
AVGGQDADLAEAPFQISLLK

SEQ ID NO: 31 - protein, 213 aa, Dermatophagoides farinae
MMKFLLIAAVAFVAVSADPIHYDKITEEINKAIDDAIAAIEQSETIDPMKVPDHADKFERHVGIVDFKGELAMRNIEARGLKQMKRQGDANVKGEEGIVKAHLLIGVHDDIVSMEYDLAYKLGDLHPTTHVISDIQDFVVALSLEISDEGNITMTSFEVRQFANVVNHIGGLSILDPIFGVLSDVLTAIFQDTVRKEMTKVLAPAFKRELEKN

SEQ ID NO: 32 - protein, 109 aa, Felis catus
MRGALLVLALLVTQALGVKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 33 - protein, 88 aa, Felis catus
MLDAALPPCPTVAATADCEICPAVKRDVDLFLTGTPDEYVEQVAQYKALPVVLENARILKNCVDAKMTEEDKENALSLLDKIYTSPLC

SEQ ID NO: 34 - protein, 92 aa, Felis catus
MKGARVLVLLWAALLLIWGGNCEICPAVKRDVDLFLTGTPDEYVEQVAQYKALPVVLENARILKNCVDAKMTEEDKENALSLLDKIYTSPLC

SEQ ID NO: 35 - protein, 138 aa, Hevea brasiliensis
MAEDEDNQQGQGEGLKYLGFVQDAATYAVTTFSNVYLFAKDKSGPLQPGVDIIEGPVKNVAVPLYNRFSYIPNGALKFVDSTVVASVTIIDRSLPPIVKDASIQVVSAIRAAPEAARSLASSLPGQTKILAKVFYGEN

SEQ ID NO: 36 - protein, 204 aa, Hevea brasiliensis
MAEEVEEERLKYLDFVRAAGVYAVDSFSTLYLYAKDISGPLKPGVDTIENVVKTVVTPVYYIPLEAVKFVDKTVDVSVTSLDGVVPPVIKQVSAQTYSVAQDAPRIVLDVASSVFNTGVQEGAKALYANLEPKAEQYAVITWRALNKLPLVPQVANVVVPTAVYFSEKYNDVVRGTTEQGYRVSSYLPLLPTEKITKVFGDEAS

SEQ ID NO: 37 - protein, 263 aa, Lolium perenne
MASSSSVLLVVALFAVFLGSAHGIAKVPPGPNITAEYGDKWLDAKSTWYGKPTGAGPKDNGGACGYKNVDKAPFNGMTGCGNTPIFKDGRGCGSCFEIKCTKPESCSGEAVTVTITDDNEEPIAPYHFDLSGHAFGSMAKKGEEQNVRSAGELELQFRRVKCKYPDDTKPTFHVEKASNPNYLAILVKYVDGDGDVVAVDIKEKGKDKWIELKESWGAVWRIDTPDKLTGPFTVRYTTEGGTKSEFEDVIPEGWKADTSYSAK

SEQ ID NO: 38 - protein, 97 aa, Lolium perenne
AAPVEFTVEKGSDEKNLALSIKYNKEGDSMAEVELKEHGSNEWLALKKNGDGVWEIKSDKPLKGPFNFRFVSEKGMRNVFDDVVPADFKVGTTYKPE

SEQ ID NO: 39 - protein, 97 aa, Lolium perenne
TKVDLTVEKGSDAKTLVLNIKYTRPGDTLAEVELRQHGSEEWEPMTKKGNLWEVKSAKPLTGPMNFRFLSKGGMKNVFDEVIPTAFTVGKTYTPEYN

SEQ ID NO: 40 - protein, 308 aa, Lolium perenne
MAVQKYTVALFLRRGPRGGPGRSYAADAGYTPAAAATPATPAATPAGGWREGDDRRAEAAGGRQRLASRQPWPPLPTPLRRTSSRSSRPPSPSPPRASSPTSAAKAPGLIPKLDTAYDVAYKAAEAHPRGQVRRLRHCPHRSLRVIAGALEVHAVKPATEEVLAAKIPTGELQIVDKIDAAFKIAATAANAAPTNDKFTVFESAFNKALNECTGGAMRPTSSSPPSRPRSSRPTPPPSPAAPEVKYAVFEAALTKAITAMTQAQKAGKPAAAAATAAATVATAAATAAAVLPPPLLVVQSLISLLIYY

SEQ ID NO: 41 - protein, 339 aa, Lolium perenne
MAVQKHTVALFLAVALVAGPAASYAADAGYAPATPATPAAPATAATPATPATPATPAAVPSGKATTEEQKLIEKINAGFKAAVAAAAVVPPADKYKTFVETFGTATNKAFVEGLASGYADQSKNQLTSKLDAALKLAYEAAQGATPEAKYDAYVATLTEALRVIAGTLEVHAVKPAAEEVKVGAIPAAEVQLIDKVDAAYRTAATAANAAPANDKFTVFENTFNNAIKVSLGAAYDSYKFIPTLVAAVKQAYAAKQATAPEVKYTVSETALKKAVTAMSEAEKEATPAAAATATPTPAAATATATPAAAYATATPAAATATATPAAATATPAAAGGYKV

SEQ ID NO: 42 - protein, 339 aa, Lolium perenne; identical to SEQ ID NO: 41

SEQ ID NO: 43 - protein, 134 aa, Lolium perenne
  misc_feature: X is unknown amino acid
DKGPGFVVTGRVYCDPCRAGFETNVSHNVEGATVAVDCRPFDGGESKLKAEATTDKDGWYKIEIDQDHQEEICEVVLAKSPDKSCSEIEEFRDRARVPLTSNXGIKQQGIRYANPIAFFRKEPLKECGGILQAY

SEQ ID NO: 44 - protein, 145 aa, Olea europaea
EDIPQPPVSQFHIQGQVYCDTCRAGFITELSEFIPGASLRLQCKDKENGDVTFTEVGYTRAEGLYSMLVERDHKNEFCEITLISSGRKDCNEIPTEGWAKPSLKFKLNTVNGTTRTVNPLGFFKKEALPKCAQVYNKLGMYPPNM

SEQ ID NO: 45 - protein, 133 aa, Parietaria judaica
MRTVSMAALVVIAAALAWTSSAEPAPAPAPGEEACGKVVQDIMPCLHFVKGEEKEPSKECCSGTKKLSEEVKTTEQKREACKCIVRATKGISGIKNELVAEVPKKCDIKTTLPPITADFDCSKIQSTIFRGYY

SEQ ID NO: 46 - protein, 133 aa, Parietaria judaica
MVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 47 - protein, 133 aa, Parietaria judaica
MRTVSMAALVVIAAALAWTSSAELASAPAPGEGPCGKVVHHIMPCLKFVKGEEKEPSKSCCSGTKKLSEEVKTTEQKREACKCIVAATKGISGIKNELVAEVPKKCGITTTLPPITADFDCSKIESTIFRGYY

SEQ ID NO: 48 - protein, 176 aa, Parietaria judaica
MRTVSAPSAVALVVIVAAGLAWTSLASVAPPAPAPGSEETCGTVVRALMPCLPFVQGKEKEPSKGCCSGAKRLDGETKTGLQRVHACECIQTAMKTYSDIDGKLVSEVPKHCGIVDSKLPPIDVNMDCKTLGVVPRQPQLPVSLRHGPVTGPSDPAHKARLERPQIRVPPPAPEKA

SEQ ID NO: 49 - protein, 138 aa, Parietaria judaica
MRTVSARSSVALVVIVAAVLVWTSSASVAPAPAPGSEETCGTVVGALMPCLPFVQGKEKEPSKGCCSGAKRLDGETKTGPQRVHACECIQTAMKTYSDIDGKLVSEVPKHCGIVDSKLPPIDVNMDCKTLGVLHYKGN

SEQ ID NO: 50 - protein, 143 aa, Parietaria judaica
MVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSRSRPPTKHGWRDPRLEFRPPHRKKPNPAFSTLG

SEQ ID NO: 51 - protein, 263 aa, Phleum pratense
MASSSSVLLVVVLFAVFLGSAYGIPKVPPGPNITATYGDKWLDAKSTWYGKPTGAGPKDNGGACGYKDVDKPPFSGMTGCGNTPIFKSGRGCGSCFEIKCTKPEACSGEPVVVHITDDNEEPIAPYHFDLSGHAFGAMAKKGDEQKLRSAGELELQFRRVKCKYPEGTKVTFHVEKGSNPNYLALLVKYVNGDGDVVAVDIKEKGKDKWIELKESWGAIWRIDTPDKLTGPFTVRYTTEGGTKTEAEDVIPEGWKADTSYESK

SEQ ID NO: 52 - protein, 262 aa, Phleum pratense
MASSSSVLLVVALFAVFLGSAHGIPKVPPGPNITATYGDKWLDAKSTWYGKPTAAGPKDNGGACGYKDVDKPPFSGMTGCGNTPIFKSGRGCGSCFEIKCTKPEACSGEPVVVHITDDNEEPIAAYHFDLSGIAFGSMAKKGDEQKLRSAGEVEIQFRRVKCKYPEGTKVTFHVEKGSNPNYLALLVKFSGDGDVVAVDIKEKGKDKWIALKESWGAIWRIDTPEVLKGPFTVRYTTEGGTKARAKDVIPEGWKADTAYESK

SEQ ID NO: 53 - protein, 122 aa, Phleum pratense
MSMASSSSSSLLAMAVLAALFAGAWCVPKVTFTVEKGSNEKHLAVLVKYEGDTMAEVELREHGSDEWVAMTKGEGGVWTFDSEEPLQGPFNFRFLTEKGMKNVFDDVVPEKYTIGATYAPEE

SEQ ID NO: 54 - protein, 276 aa, Phleum pratense
ADLGYGGPATPAAPAEAAPAGKATTEEQKLIEKINDGFKAALAAAAGVPPADKYKTFVATFGAASNKAFAEGLSAEPKGAAESSSKAALTSKLDAAYKLAYKTAEGATPEAKYDAYVATLSEALRIIAGTLEVHAVKPAAEEVKVIPAGELQVIEKVDSAFKVAATAANAAPANDKFTVFEAAFNNAIKASTGGAYESYKFIPALEAAVKQAYAATVATAPEVKYTVFETALKKAFTAMSEAQKAAKPATEATATATAAVGAATGAATAATGGYKV

SEQ ID NO: 55 - protein, 276 aa, Phleum pratense
ADLGYGGPATPAAPAEAAPAGKATTEEQKLIEKINDGFKAALAAAAGVPPADKYKTFVATFGAASNKAFAEGLSAEPKGAAESSSKAALTSKLDAAYKLAYKTAEGATPEAKYDAYVATLSEALRIIAGTLEVHAVKPAAEEVKVIPAGELQVIEKVDSAFKVAATAANAAPANDKFTVFEAAFNNAIKASTGGAYESYKFIPALEAAVKQAYAATVATAPEVKYTVFETALKKAITAMSEAQKAAKPATEATATATAAVGAATGAATAATGGYKV

SEQ ID NO: 56 - protein, 284 aa, Phleum pratense
AAAAVPRRGPRGGPGRSYTADAGYAPATPAAAGAAAGKATTEEQKLIEDINVGFKAAVAAAASVPAADKFKTFEAAFTSSSKAAAAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 57 - protein, 286 aa, Phleum pratense
ADLGYGPATPAAPAAGYTPATPAAPAGADAAGKATTEEQKLIEKINAGFKAALAGAGVQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDEIKASTGGAYESYKFIPALEAAVKQAYAATVATAPEVKYTVFETALKKAITAMSEAQKAAKPAAAATATATAAVGAATGAATAATGGYKV

SEQ ID NO: 58 - protein, 287 aa, Phleum pratense
MAVQKYTVALFLAVALVAGPAASYAADAGYAPATPAAAGAEAGKATTEEQKLIEDINVGFKAAVAAAASVPAADKFKTFEAAFTSSSKAATAKAPGLVPKLDAAYSVSYKAAVGATPEAKFDSFVASLTEALRVIAGALEVHAVKPVTEEPGMAKIPAGELQIIDKIDAAFKVAATAAATAPADTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 59 - protein, 290 aa, Phleum pratense
MAVQKYTVALFLAVALVAGPAASYAADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEDPAWPKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTATGAASGAATVAAGGYKV

SEQ ID NO: 60 - protein, 265 aa, Phleum pratense
ADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 61 - protein, 295 aa, Phleum pratense
SVKRSNGSAEVHRGAVPRRGPRGGPGRSYAADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 62 - protein, 312 aa, Phleum pratense
MAVHQYTVALFLAVALVAGPAGSYAADLGYGPATPAAPAAGYTPATPAAPAGAEPAGKATTEEQKLIEKINAGFKAALAAAAGVPPADKYRTFVATFGAASNKAFAEGLSGEPKGAAESSSKAALTSKLDAAYKLAYKTAEGATPEAKYDAYVATVSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 63 - protein, 276 aa, Phleum pratense; identical to SEQ ID NO: 54

SEQ ID NO: 64 - protein, 284 aa, Phleum pratense; identical to SEQ ID NO: 56

SEQ ID NO: 65 - protein, 286 aa, Phleum pratense; identical to SEQ ID NO: 57

SEQ ID NO: 66 - protein, 281 aa, Phleum pratense
AVPRRGPRGGPGRSYAADAGYAPATPAAAGAEAGKATTEEQKLIEDINVGFKAAVAAAASVPAGDKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTATGAASGAATVAAGGYKV

SEQ ID NO: 67 - protein, 280 aa, Phleum pratense
MAVPRRGPRGGPGRSYTADAGYAPATPAAAGAAAGKATTEEQKLIEDINVGFKAAVAARQRPAADKFKTFEAASPRHPRPLRQGAGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAAEVKYAVFEAALTKAITAMSEVQKVSQPATGAATVAAGAATTAAGAASGAATVAAGGYKV

SEQ ID NO: 68 - protein, 312 aa, Phleum pratense
MAVHQYTVALFLAVALVAGPAASYAADLGYGPATPAAPAAGYTPATPAAPAEAAPAGKATTEEQKLIEKINAGFKAALAAAAGVQPADKYRTFVATFGAASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 69 - protein, 257 aa, Phleum pratense
EAPAGKATTEEQKLIEKINAGFKAALARRLQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAAELQVIEKVDAAFKVAATAANAAPANDKFTVFEAAFNDEIKASTGGAYESYKFIPALEAAVKQAYAATVATAPEVKYTVFETALKKAITAMSEAQKAAKPPPLPPPPQPPPLAATGAATAATGGYKV

SEQ ID NO: 70 - protein, 312 aa, Phleum pratense; identical to SEQ ID NO: 68

SEQ ID NO: 71 - protein, 280 aa, Phleum pratense; identical to SEQ ID NO: 67

SEQ ID NO: 72 - protein, 285 aa, Phleum pratense
ADLGYGPATPAAPAAGYTPATPAAPAGADAAGKATTEEQKLIEKINAGFKAALAGAGVQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDEIKASTGGAYESYKFIPALEAAVKQAYAATVATAPEVKYTVFETALKKAITAMSEAQKAAKPPPLPPPPQPPPLAATGAATAATGGYKV

SEQ ID NO: 73 - protein, 312 aa, Phleum pratense; identical to SEQ ID NO: 68

SEQ ID NO: 74 - protein, 138 aa, Phleum pratense
MAAHKFMVAMFLAVAVVLGLATSPTAEGGKATTEEQKLIEDVNASFRAAMATTANVPPADKYKTFEAAFTVSSKRNLADAVSKAPQLVPKLDEVYNAAYNAADHAAPEDKYEAFVLHFSEALRIIAGTPEVHAVKPGA

SEQ ID NO: 75 - protein, 57 aa, Phleum pratense
SKAPQLVPKLDEVYNAAYNAADHAAPEDKYEAFVLHFSEALHIIAGTPEVHAVKPGA

SEQ ID NO: 76 - protein, 80 aa, Phleum pratense
ADKYKTFEAAFTVSSKRNLADAVSKAPQLVPKLDEVYNAAYNAADHAAPEDKYEAFVLHFSEALHIIAGTPEVHAVKPGA

SEQ ID NO: 77 - protein, 106 aa, Phleum pratense
TEEQKLIEDVNASFRAAMATTANVPPADKYKTLEAAFTVSSKRNLADAVSKAPQLVPKLDEVYNAAYNAADHAAPEDKYEAFVLHFSEALRIIAGTPEVHAVKPGA

SEQ ID NO: 78 - protein, 138 aa, Phleum pratense
MAAHKFMVAMFLAVAVVLGLATSPTAEGGKATTEEQKLIEDINASFRAAMATTANVPPADKYKTFEAAFTVSSKRNLADAVSKAPQLVPKLDEVYNAAYNAADHAAPEDKYEAFVLHFSEALHIIAGTPEVHAVKPGA

SEQ ID NO: 79 - protein, 132 aa, Phleum pratense
MVAMFLAVAVVLGLATSPTAEGGKATTEEQKLIEDVNASFRAAMATTANVPPADKYKTFEAAFTVSSKRNLADAVSKAPQLVPKLDEVYNAAYNAADHAAPEDKYEAFVLHFSEALRIIAGTPEVHAVKPGA

SEQ ID NO: 80 - protein, 78 aa, Phleum pratense
MADDMERIFKRFDTNGDGKISLSELTDALRTLGSTSADEVQRMMAEIDTDGDGFIDFNEFISFCNANPGLMKDVAKVF

SEQ ID NO: 81 - protein, 131 aa, Phleum pratense
MSWQTYVDEHLMCEIEGHHLASAAILGHDGTVWAQSADFPQFKPEEITGIMKDFDEPGHLAPTGMFVAGAKYMVIQGEPGRVIRGKKGAGGITIKKTGQALVVGIYDEPMTPGQCNMVVERLGDYLVEQGM

SEQ ID NO: 82 - protein, 227 aa, Vespula vulgaris
MEISGLVYLIIIVTIIDLPYGKANNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFMNEELYQTK

SEQ ID NO: 83 - protein, 300 aa, Vespula maculifrons
GPKCPFNSDTVSIIIETRENRNRDLYTLQTLQNHPEFKKKTITRPVVFITHGFTSSASEKNFINLAKALVDKDNYMVISIDWQTAACTNEYPGLKYAYYPTAASNTRLVGQYIATITQKLVKDYKISMANIRLIGHSLGAHVSGFAGKRVQELKLGKYSEIIGLDPARPSFDSNHCSERLCETDAEYVQIIHTSNYLGTEKILGTVDFYMNNGKNNPGCGRFFSEVCSHTRAVIYMAECIKHECCLIGIPRSKSSQPISRCTKQECVCVGLNAKKYPSRGSFYVPVESTAPFCNNKGKII

SEQ ID NO: 84 - protein, 336 aa, Vespula vulgaris
MEENMNLKYLLLFVYFVQVLNCCYGHGDPLSYELDRGPKCPFNSDTVSIIIETRENRNRDLYTLQTLQNHPEFKKKTITRPVVFITHGFTSSASETNFINLAKALVDKDNYMVISIDWQTAACTNEAAGLKYLYYPTAARNTRLVGQYIATITQKLVKHYKISMANIRLIGHSLGAHASGFAGKKVQELKLGKYSEIIGLDPARPSFDSNHCSERLCETDAEYVQIIHTSNYLGTEKTLGTVDFYMNNGKNQPGCGRFFSEVCSHSRAVIYMAECIKHECCLIGIPKSKSSQPISSCTKQECVCVGLNAKKYPSRGSFYVPVESTAPFCNNKGKII

SEQ ID NO: 85 - protein, 331 aa, Vespula vulgaris
SERPKRVFNIYWNVPTFMCHQYDLYFDEVTNFNIKRNSKDDFQGDKIAIFYDPGEFPALLSLKDGKYKKRNGGVPQEGNITIHLQKFIENLDKIYPNRNFSGIGVIDFERWRPIFRQNWGNMKIHKNFSIDLVRNEHPTWNKKMIELEASKRFEKYARFFMEETLKLAKKTRKQADWGYYGYPYCFNMSPNNLVPECDVTAMHENDKMSWLFNNQNVLLPSVYVRQELTPDQRIGLVQGRVKEAVRISNNLKHSPKVLSYWWYVYQDETNTFLTETDVKKTFQEIVINGGDGIIIWGSSSDVNSLSKCKRLQDYLLTVLGPIAINVTEAVN

SEQ ID NO: 86 - protein, 206 aa, Vespula vidua
KVNYCKIKCLKGGVHTACKYGTSTKPNCGKMVVKAYGLTEAEKQEILKVHNDFRQKVAKGLETRGNPGPQPPAKNMNNLVWNDELANIAQVWASQCNYGHDTCKDTEKYPVGQNIAKRSTTAALFDSPGKLVKMWENEVKDFNPNIEWSKNNLKKTGHYTQMVWAKTKEIGCGSVKYVKDEWYTHYLVCNYGPSGNFRNEKLYEKK

SEQ ID NO: 87 - protein, 160 aa, Betula pendula
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 88 - protein, 133 aa, Betula pendula
MSWQTYVDEHLMCDIDGQASNSLASAIVGHDGSVWAQSSSFPQFKPQEITGIMKDFEEPGHLAPTGLHLGGIKYMVIQGEAGAVIRGKKGSGGITIKKTGQALVFGIYEEPVTPGQCNMVVERLGDYLIDQGL

SEQ ID NO: 89 - protein, 205 aa, Betula pendula
MPCSTEAMEKAGHGHASTPRKRSLSNSSFRLRSESLNTLRLRRIFDLFDKNSDGIITVDELSRALNLLGLETDLSELESTVKSFTREGNIGLQFEDFISLHQSLNDSYFAYGGEDEDDNEEDMRKSILSQEEADSFGGFKVFDEDGDGYISARELQMVLGKLGFSEGSEIDRVEKMIVSVDSNRDGRVDFFEFKDMMRSVLVRSS

SEQ ID NO: 90 - protein, 85 aa, Betula pendula
MADDHPQDKAERERIFKRFDANGDGKISAAELGEALKTLGSITPDEVKHMMAEIDTDGDGFISFQEFTDFGRANRGLLKDVAKIF

SEQ ID NO: 91 - protein, 24 aa, Quercus alba
  misc_feature: X is unknown amino acid
GVFTXESQETSVIAPAXLFKALFL

SEQ ID NO: 92 - protein, 40 aa, Carpinus betulus
  misc_feature: X is unknown amino acid
GVFNYEAETPSVIPAARLFKSYVLDGDKLIPKVAPQAIXK

SEQ ID NO: 93 - protein, 44 aa, Alnus glutinosa
GVFNYEAETPSVIPAARLFKAFILDGDKLLPKVAPEAVSSVENI

SEQ ID NO: 94 - protein, 110 aa, Betula pendula
VQCMQVWPPLGLKKFETLSYLPPLSSEQLAKEVDYLLRKNLIPCLEFELEHGFVYREHNRSPGYYDGRYWTMWKLPMFGCNDSSQVLKELEECKKAYPSAFIRIIGFDDK

SEQ ID NO: 95 - protein, 626 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 96 - protein, 392 aa, Ambrosia artemisiifolia
MGIKHCCYILYFTLALVTLLQPVRSAEDLQQILPSANETRSLTTCGTYNIIDGCWRGKADWAENRKALADCAQGFAKGTIGGKDGDIYTVTSELDDDVANPKEGTLRFGAAQNRPLWIIFARDMVIRLDRELAINNDKTIDGRGAKVEIINAGFAIYNVKNIIIHNIIMHDIVVNPGGLIKSHDGPPVPRKGSDGDAIGISGGSQIWIDHCSLSKAVDGLIDAKHGSTHFTVSNCLFTQHQYLLLFWDFDERGMLCTVAFNKFTDNVDQRMPNLRHGFVQVVNNNYERWGSYALGGSAGPTILSQGNRFLASDIKKEVVGRYGESAMSESINWNWRSYMDVFENGAIFVPSGVDPVLTPEQNAGMIPAEPGEAVLRLTSSAGVLSCQPGAPC

SEQ ID NO: 97 - protein, 397 aa, Ambrosia artemisiifolia
MGIKHCCYILYFTLALVTLVQAGRLGEEVDILPSPNDTRRSLQGCEAHNIIDKCWRCKPDWAENRQALGNCAQGFGKATHGGKWGDIYMVTSDQDDDVVNPKEGTLRFGATQDRPLWIIFQRDMIIYLQQEMVVTSDKTIDGRGAKVELVYGGITLMNVKNVIIHNIDIHDVRVLPGGRIKSNGGPAIPRHQSDGDAIHVTGSSDIWIDHCTLSKSFDGLVDVNWGSTGVTISNCKFTHHEKAVLLGASDTHFQDLKMHVTLAYNIFTNTVHERMPRCRFGFFQIVNNFYDRWDKYAIGGSSNPTILSQGNKFVAPDFIYKKNVCLRTGAQEPEWMTWNWRTQNDVLENGAIFVASGSDPVLTAEQNAGMMQAEPGDMVPQLTMNAGVLTCSPGAPC

SEQ ID NO: 98 - protein, 397 aa, Ambrosia artemisiifolia
MGIKQCCYILYFTLALVALLQPVRSAEGVGEILPSVNETRSLQACEALNIIDKCWRGKADWENNRQALADCAQGFAKGTYGGKWGDVYTVTSNLDDDVANPKEGTLRFAAAQNRPLWIIFKNDMVINLNQELVVNSDKTIDGRGVKVEIINGGLTLMNVKNIIIHNINIHDVKVLPGGMIKSNDGPPILRQASDGDTINVAGSSQIWIDHCSLSKSFDGLVDVTLGSTHVTISNCKFTQQSKAILLGADDTHVQDKGMLATVAFNMFTDNVDQRMPRCRFGFFQVVNNNYDRWGTYAIGGSSAPTILCQGNRFLAPDDQIKKNVLARTGTGAAESMAWNWRSDKDLLENGAIFVTSGSDPVLTPVQSAGMIPAEPGEAAIKLTSSAGVFSCHPGAPC

SEQ ID NO: 99 - protein, 398 aa, Ambrosia artemisiifolia
MGIKHCCYILYFTLALVTLLQPVRSAEDVEEFLPSANETRRSLKACEAHNIIDKCWRCKADWANNRQALADCAQGFAKGTYGGKHGDVYTVTSDKDDDVANPKEGTLRFAAAQNRPLWIIFKRNMVIHLNQELVVNSDKTIDGRGVKVNIVNAGLTLMNVKNIIIHNINIHDIKVCPGGMIKSNDGPPILRQQSDGDAINVAGSSQIWIDHCSLSKASDGLLDITLGSSHVTVSNCKFTQHQFVLLLGADDTHYQDKGMLATVAFNMFTDHVDQRMPRCRFGFFQVVNNNYDRWGTYAIGGSSAPTILSQGNRFFAPDDIIKKNVLARTGTGNAESMSWNWRTDRDLLENGAIFLPSGSDPVLTPEQKAGMIPAEPGEAVLRLTSSAGVLSCHQGAPC

SEQ ID NO: 100 - protein, 396 aa, Ambrosia artemisiifolia
MGIKHCCYILYFTLALVTLLQPVRSAEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 101 - protein, 373 aa, Cryptomeria japonica
MDSPCLVALLVFSFVIGSCFSDNPIDSCWRGDSNWAQNRMKLADCAVGFGSSTMGGKGGDLYTVTNSDDDPVNPPGTLRYGATRDRPLWIIFSGNMNIKLKMPMYIAGYKTFDGRGAQVYIGNGGPCVFIKRVSNVIIHGLYLYGCSTSVLGNVLINESFGVEPVHPQDGDALTLRTATNIWIDHNSFSNSSDGLVDVTLTSTGVTISNNLFFNHHKVMSLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPHLTQNAGVLTCSLSKRC

SEQ ID NO: 102 - protein, 374 aa, Cryptomeria japonica
MDSPCLVALLVLSFVIGSCFSDNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRC

SEQ ID NO: 103 - protein, 514 aa, Cryptomeria japonica
MAMKLIAPMAFLAMQLIIMAAAEDQSAQIMLDSVVEKYLRSNRSLRKVEHSRHDAINIFNVEKYGAVGDGKHDCTEAFSTAWQAACKNPSAMLLVPGSKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKDIKLSDISLKLTSGKIASCLNDNANGYFSGHVIPACKNLSPSAKRKESKSHKHPKTVMVENMRAYDKGNRTRILLGSRPPNCTNKCHGCSPCKAKLVIVHRIMPQEYYPQRWICSCHGKIYHP

SEQ ID NO: 104 - protein, 514 aa, Cryptomeria japonica
MAMKFIAPMAFVAMQLIIMAAAEDQSAQIMLDSDIEQYLRSNRSLRKVEHSRHDAINIFNVEKYGAVGDGKHDCTEAFSTAWQAACKKPSAMLLVPGNKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKDIKLSDISLKLTSGKIASCLNDNANGYFSGHVIPACKNLSPSAKRKESKSHKHPKTVMVKNMGAYDKGNRTRILLGSRPPNCTNKCHGCSPCKAKLVIVHRIMPQEYYPQRWMCSRHGKIYHP

SEQ ID NO: 105 - protein, 373 aa, Cryptomeria japonica
MDSPCLVALLVLSFVIGSCFSDNPIDSCWRGDSNWAQNRMKLADCAVGFGSSTMGGKGGDLYTVTNSDDDPVNPPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRC

SEQ ID NO: 106 - protein, 374 aa, Cryptomeria japonica
MDSPCLVALLVFSFVIGSCFSDNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLYLYGCSTSVLGNVLINESFGVEPVHPQDGDALTLRTATNIWIDHNSFSNSSDGLVDVTLTSTGVTISNNLFFNHHKVMSLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPHLTQNAGVLTCSLSKRC

SEQ ID NO: 107 - protein, 174 aa, Canis familiaris
MKTLLLTIGFSLIAILQAQDTPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 108 - protein, 24 aa, Canis familiaris
EAYKSEIAHRYNDLGEEHFRGLVL

SEQ ID NO: 109 - protein, 265 aa, Canis familiaris
LSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVVEVSRKLGKVGTKCCKKPESERMSCADDFLS

SEQ ID NO: 110 - protein, 180 aa, Canis familiaris
MQLLLLTVGLALICGLQAQEGNHEEPQGGLEELSGRWHSVALASNKSDLIKPWGHFRVFIHSMSAKDGNLHGDILIPQDGQCEKVSLTAFKTATSNKFDLEYWGHNDLYLAEVDPKSYLILYMINQYNDDTSLVAHLMVRDLSRQQDFLPAFESVCEDIGLHKDQIVVLSDDDRCQGSRD

SEQ ID NO: 111 - protein, 187 aa, Equus caballus
MKLLLLCLGLILVCAQQEENSDVAIRNFDISKISGEWYSIFLASDVKEKIEENGSMRVFVDVIRALDNSSLYAEYQTKVNGECTEFPMVFDKTEEDGVYSLNYDGYNVFRISEFENDEHIILYLVNFDKDRPFQLFEFYAREPDVSPEIKEEFVKIVQKRGIVKENIIDLTKIDRCFQLRGNGVAQA

SEQ ID NO: 112 - protein, 29 aa, Equus caballus
  misc_feature: X is unknown amino acid
SQXPQSETDYSQLSGEWNTIYGAASNIXK

SEQ ID NO: 113 - protein, 211 aa, Euroglyphus maynei
TYACSINSVSLPSELDLRSLRTVTPIRMQGGCGSCWAFSGVASTESAYLAYRNMSLDLAEQELVDCASQNGCHGDTIPRGIEYIQQNGVVQEHYYPYVAREQSCHRPNAQRYGLKNYCQISPPDSNKIRQALTQTHTAVAVIIGIKDLNAFRHYDGRTIMQHDNGYQPNYHAVNIVGYGNTQGVDYWIVRNSWDTTWGDNGYGYFAANINL

SEQ ID NO: 114 - protein, 211 aa, Euroglyphus maynei; identical to SEQ ID NO: 113

SEQ ID NO: 115 - protein, 211 aa, Euroglyphus maynei
ETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDL

SEQ ID NO: 116 - protein, 212 aa, Euroglyphus maynei
ETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQQCRRPNSQHYGISNYCQIYPPDVKQIREALTQTHTAIAVIIGIKDLRAFQHYDGRTIIQHDNGYQPNYHAVNIVGYGSTQGVDYWIVRNSWDTTWGDSGYGYFQAGNNL

SEQ ID NO: 117 - protein, 307 aa, Poa pratensis
MAVQKYTVALFLVALVVGPAASYAADLSYGAPATPAAPAAGYTPAAPAGAAPKATTDEQKMIEKINVGFKAAVAAAGGVPAANKYKTFVATFGAASNKAFAEALSTEPKGAAVDSSKAALTSKLDAAYKLAYKSAEGATPEAKYDDYVATLSEALRIIAGTLEVHGVKPAAEEVKATPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAATGTATAAVGAATGAATAAAGGYKV

SEQ ID NO: 118 - protein, 333 aa, Poa pratensis
MAVHQYTVALFLAVALVAGPAASYAADVGYGAPATLATPATPAAPAAGYTPAAPAGAAPKATTDEQKLIEKINAGFKAAVAAAAGVPAVDKYKTFVATFGTASNKAFAEALSTEPKGAAAASSNAVLTSKLDAAYKLAYKSAEGATPEAKYDAYVATLSEALRIIAGTLEVHAVKPAGEEVKAIPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAVTATATGAVGAATGAVGAATGAATAAAGGYKTGAATPTAGGYKV

SEQ ID NO: 119 - protein, 373 aa, Poa pratensis
MDKANGAYKTALKAASAVAPAEKFPVFQATFDKNLKEGLSGPDAVGFAKKLDAFIQTSYLSTKAAEPKEKFDLFVLSLTEVLRFMAGAVKAPPASKFPAKPAPKVAAYTPAAPAGAAPKATTDEQKLIEKINVGFKAAVAAAAGVPAASKYKTFVATFGAASNKAFAEALSTEPKGAAVASSKAVLTSKLDAAYKLAYKSAEGATPEAKYDAYVATLSEALRIIAGTLEVHGVKPAAEEVKAIPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAVTGTATSAVGAATGAATAAAGGYKV

SEQ ID NO: 120 - protein, 685 aa, Periplaneta americana
MKTALVFAAVVAFVAARFPDHKDYKQLADKQFLAKQRDVLRLFHRVHQHNILNDQVEVGIPMTSKQTSATTVPPSGEAVHGVLQEGHARPRGEPFSVNYEKHREQAIMLYDLLYFANDYDTFYKTACWARDRVNEGMFMYSFSIAVFHRDDMQGVMLPPPYEVYPYLFVDHDVIHMAQKYWMKNAGSGEHHSHVIPVNFTLRTQDHLLAYFTSDVNLNAFNTYYRYYYPSWYNTTLYGHNIDRRGEQFYYTYKQIYARYFLERLSNDLPDVYPFYYSKPVKSAYNPNLRYHNGEEMPVRPSNMYVTNFDLYYIADIKNYEKRVEDAIDFGYAFDEHMKPHSLYHDVHGMEYLADMIEGNMDSPNFYFYGSIYHMYHSMIGHIVDPYHKMGLAPSLEHPETVLRDPVFYQLWKRVDHLFQKYKNRLPRYTHDELAFEGVKVENVDVGKLYTYFEQYDMSLDMAVYVNNVDQISNVDVQLAVRLNHKPFTYNIEVSSDKAQDVYVAVFLGPKYDYLGREYDLNDRRHYFVEMDRFPYHVGAGKTVIERNSHDSNIIAPERDSYRTFYKKVQEAYEGKSQYYVDKGHNYCGYPENLLIPKGKKGGQAYTFYVIVTPYVKQDEHDFEPYNYKAFSYCGVGSERKYPDNKPLGYPFDRKIYSNDFYTPNMYFKDVIIFHKKYDEVGVQGH

SEQ ID NO: 121 - protein, 446 aa, Periplaneta americana
INEIHSIIGLPPFVPPSRRHARRGVGINGLIDDVIAILPVDELKALFQEKLETSPDFKALYDAIRSPEFQSIISTLNAMQRSEHHQNLRDKGVDVDHFIQLIRALFGLSRAARNLQDDLNDFLHSLEPISPRHRHGLPRQRRRSARVSAYLHADDFHKIITTIEALPEFANFYNFLKEHGLDVVDYINEIHSIIGLPPFVPPSRRHARRGVGINGLIDDVIAILPVDELKALFQEKLETSPDFKALYDAIRSPEFQSIISTLNAMPEYQELLQNLRDKGVDVDHFIRVDQGTLRTLSSGQRNLQDDLNDFLALIPTDQILAIAMDYLANDAEVQELVAYLQSDDFHKIITTIEALPEFANFYNFLKEHGLDVVDYINEIHSIIGLPPFVPPSQRHARRGVGINGLIDDVIAILPVDELKALFQEKLETSPDFKALYDAIDLRSSRA

SEQ ID NO: 122 - protein, 352 aa, Blattella germanica
MIGLKLVTVLFAVATITHAAELQRVPLYKLVHVFINTQYAGITKIGNQNFLTVFDSTSCNVVVASQECVGGACVCPNLQKYEKLKPKYISDGNVQVKFFDTGSAVGRGIEDSLTISNLTTSQQDIVLADELSQEVCILSADVVVGIAAPGCPNALKGKTVLENFVEENLIAPVFSIHHARFQDGEHFGEIIFGGSDWKYVDGEFTYVPLVGDDSWKFRLDGVKIGDTTVAPAGTQAIIDTSKAIIVGPKAYVNPINEAIGCVVEKTTTRRICKLDCSKIPSLPDVTFVINGRNFNISSQYYIQQNGNLCYSGFQPCGHSDHFFIGDFFVDHYYSEFNWENKTMGFGRSVESV

SEQ ID NO: 123 - protein, 182 aa, Blattella germanica
AVLALCATDTLANEDCFRHESLVPNLDYERFRGSWIIAAGTSEALTQYKCWIDRFSYDDALVSKYTDSQGKNRTTIRGRTKFEGNKFTIDYNDKGKAFSAPYSVLATDYENYAIVEGCPAAANGHVIYVQIRFSVRRFHPKLGDKEMIQHYTLDQVNQHKKAIEEDLKHFNLKYEDLHSTCH

SEQ ID NO: 124 - protein, 200 aa, Blattella germanica
YKLTYCPVKALGEPIRFLLSYGEKDFEDYRFQEGDWPNLKPSMPFGKTPVLEIDGKQTHQSVAISRYLGKQFGLSGKDDWENLEIDMIVDTISDFRAAIANYHYDADENSKQKKWDPLKKETIPYYTKKFDEVVKANGGYLAAGKLTWADFYFVAILDYLNHMAKEDLVANQPNLKALREKVLGLPAIKAWVAKRPPTDL

(19) United States
(12) Patent Application Publication
(10) Pub. No.: US 2004/0265342 A1
(43) Pub. Date: Dec. 30, 2004

(54) Methods and compositions for desensitisation

(75) Inventors: Mark Larche, Surrey, London (GB); Anthoney Barrington Kay, London (GB)

Correspondence Address: PALMER & DODGE, LLP, KATHLEEN M. WILLIAMS, 111 HUNTINGTON AVENUE, BOSTON, MA, 02199, US

(73) Assignee: Circassia Limited

(21) Appl. No.: 10/809,689

(22) Filed: Mar. 25, 2004

Related U.S. Application Data
(63) Continuation of application No. 09/610,134, filed on Jul. 5, 2000, now abandoned.
Application No. 09/610,134 is a continuation of application No. PCT/GB99/00080, filed on Jan. 11, 1999.

(30) Foreign Application Priority Data
Jan. 9, 1998 (GB) 9800445.0
Sep. 21, 1998 (GB) 9820474.6

(51) Int. Cl. A61K 39/35

(52) U.S. Cl. 424/275.1

(57) ABSTRACT

The invention relates to a method of desensitising a patient to a polypeptide allergen the method comprising administering to the patient a peptide derived from the allergen wherein restriction to a MHC Class II molecule possessed by the patient can be demonstrated by the peptide and the peptide is able to induce a late phase response in an individual who possesses the said MHC Class II molecule. A composition comprising a plurality of peptides derived from a polypeptide allergen wherein for at least one of the peptides in the composition restriction to a MHC Class II molecule can be demonstrated, and the composition is able to induce a late phase response in an individual possessing the given MHC Class II moleculeis also provided. The invention also relates to a method of selecting a peptide for use as an immunotherapeutic agent for desensitising a patient to a polypeptide allergen capable of eliciting an allergic response in the patient.

The present invention relates to methods and compositions for desensitising patients who are hypersensitive to particular allergens, especially polypeptide allergens. Moreover, the invention relates to immunological vaccines which may be used to prevent and/or treat conditions involving hypersensitivity to allergens.

The ability of the immune system to elicit a response to a particular molecule depends critically upon its ability to recognise the presence of an antigen. Classically, the term antigen has been associated with the ability of a molecule to be an antibody generator via induction of B-cells. It is now known, however, that T cells also possess the ability to recognise antigens. T-cell antigen recognition requires antigen presenting cells (APCs) to present antigen fragments (peptides) on their cell surface in association with molecules of the major histocompatibility complex (MHC). T cells use their antigen specific T-cell receptors (TCRs) to recognise the antigen fragments presented by the APC. Such recognition acts as a trigger to the immune system to generate a range of responses to eradicate the antigen which has been recognised.

T lymphocytes have been implicated in the pathogenesis of a wide variety of diseases involving immune recognition of antigens derived both from the internal (host) and external environments. Autoimmune diseases such as autoimmune thyroiditis, rheumatoid arthritis and lupus erythrematosus arise from the recognition by the immune system of host, or self, antigens.

Recognition of external antigens by the immune system of an organism, such as man, can in some cases result in diseases, known as atopic conditions. An example of the latter are the allergic diseases including asthma, atopic dermatitis and allergic rhinitis. In this group of diseases, B lymphocytes generate antibodies of the IgE class (in humans) which bind externally derived antigens, which are referred to in this context as allergens, since these molecules elicit an allergic response. Production of allergen-specific IgE is dependent upon T lymphocytes which are also activated by (are specific for) the allergen. Allergen-specific IgE antibodies bind to the surface of cells such as basophils and mast cells by virtue of the expression by these cells of surface receptors for IgE. Crosslinking of surface bound IgE molecules by allergen results in degranulation of these effector cells causing release of inflammatory mediators such as histamine, 5-hydroxtryptamine and lipid mediators such as the sulphidoleukotrienes. In addition to IgE-dependent events, certain allergic diseases such as asthma are characterised by IgE-independent events. It has been demonstrated that the induction of the late phase reaction is an IgE-independent event which is dependent upon the activation of allergen-specific T lymphocytes.

Allergic IgE-mediated diseases are currently treated with agents which provide symptomatic relief or prevention. Examples of such agents are anti-histamines, β₂ agonists, and glucocorticosteroids. In addition, some IgE-mediated diseases are treated by desensitisation procedures that involve the periodic injection of allergen components or extracts. Desensitisation treatments may induce an IgG response that competes with IgE for allergen, or they may induce specific suppressor T cells that block the synthesis of IgE directed against allergen. This form of treatment is not always effective and poses the risk of provoking serious side effects, particularly general anaphylactic shock. This can be fatal unless recognised immediately and treated with adrenaline. A therapeutic treatment that would decrease or eliminate the unwanted allergic-immune response to a particular allergen, without altering the immune reactivity to other foreign antigens or triggering an allergic response itself would be of great benefit to allergic individuals.

Asthma can be provoked by inhalation of allergen in the clinical laboratory under controlled conditions. The response is characterised by an early asthmatic reaction (EAR) followed by a delayed-in-time late asthmatic reaction (LAR) (See Allergy and Allergic Diseases (1997), A. B. Kay (Ed.), Blackwell Science, pp 1113 to 1130). The EAR occurs within minutes of exposure to allergen, is maximal between 10 and 15 min and usually returns to near baseline by 1 hour. It is generally accepted that the EAR is dependent on the IgE-mediated release of mast cell-derived mediators such as histamine and leukotrienes. In contrast the LAR reaches a maximum at 6-9 hours and is believed to represent, at least in part, the inflammatory component of the asthmatic response and in this sense has served as a useful model of chronic asthma.

The late asthmatic response is typical of responses to allergic stimuli collectively known as late phase responses (LPR). LPR is seen particularly in the skin and the nose following intracutaneous or intranasal administration of allergens.

Using cat allergic individuals (rhinitic and asthmatic), Norman et al (1996) Am. J. Respir. Crit. Care Med. 154:1623-8 attempted to induce the counterpart of murine experimental T cell tolerance by subcutaneous injection of “T cell reactive peptides” (termed IPC1 and IPC2) in humans. Peptides were designed on the basis of patterns of epitope recognition of short overlapping peptides by Fel d 1 reactive T cell lines. It was found that peptides derived from chain 1 gave greater proliferative responses than chain 2, with the majority of activity being associated in the N terminal region of chain 1. IPC1 and IPC2 were considerably longer (27 amino acids each) than previously defined T-cell epitopes. This may have been partly responsible for immediate (presumed IgE-mediated) reactions in some patients following administration (Norman et al, Op. Cit.). Large peptide doses (4×750 μg) were required to achieve minimal clinical efficacy. The choice of peptides for therapy was based upon reactivity of secondary T-cell lines derived from a large number of cat-allergic individuals and did not take into account primary T-cell reactivity (ie ex vivo), which may be more sensitive, or MHC class II haplotype.

Norman et al reported a number of adverse hypersensitivity reactions including respiratory, and other allergic, symptoms. As stated, some had a rapid time of onset ie with 10 minutes whereas others were not observed until several hours after IPC1/IPC2 administration (although there was no local redness or swelling at the site of injection). These results have been interpreted as indicating the unsuitability of the peptides for immunotherapy, the production of a LPR being considered to be undesirable (Wheeler & Drachenberg (1997) Allergy 52:602-612).

WO 92/11859 describes a method of reducing the immune response to an allergen in which a non-allergen derived, non-stimulating peptide which binds to specific MHC class II molecules of APCs is used to inhibit T-cell response to particular allergens.

WO 91/06571 purports to disclose peptides derived from human T-cell reactive feline protein which can be used in the diagnosis, treatment or prevention of cat allergy.

WO 94/24281 relates to peptides and modified peptides of the major house dust mite allergens. The modified peptides have the intent of reducing the level of undesirable side effects associated with desensitising therapies.

We have observed that peptide allergens used in immunotherapy associate with particular MHC types in patients. Moreover, successful desensitisation of patients is achieved where a peptide allergen is used which is capable of giving an initial LPR in an individual to whom it is administered.

The MHC complex is a genetic locus made up of a number of genes which encode MHC molecules. MHC molecules are also known as Human Leucocyte Antigens (HLA).

Each individual inherits a number of MHC genes from each parent and the genes are referred to collectively as the individual's haplotype. This is a genetic term referring to the genes rather than the molecules they encode. Although the term “haplotype” should, strictly speaking, be used to describe the genes inherited from one parent, it is generally used to include genes from both sets of parents. Where the term is used in this patent specification it is given this general meaning unless the context suggests the stricter meaning.

A first aspect of the invention provides a method of desensitising a patient to a polypeptide allergen the method comprising administering to the patient a peptide derived from the allergen wherein restriction to a MHC Class II molecule possessed by the patient can be demonstrated for the peptide and the peptide is able to induce a late phase response in an individual who possesses the said MHC Class II molecule.

Restriction to a MHC Class II molecule possessed by the patient can be demonstrated for the peptide by, for example, T cell reactivity to the peptide. By “MHC Class II molecule possessed by the patient” is meant the particular type which type, of course, may be possessed by other individuals which have the genes that encode the particular type of MHC Class II molecule.

By a “peptide derived from the allergen” we include the meaning that the peptide is chemically derived from the polypeptide allergen, for example by proteolytic cleavage and we also include the meaning that the peptide is derived in an intellectual sense from the polypeptide allergen, for example by making use of the amino acid sequence of the polypeptide allergen and synthesising peptides based on the sequence. Peptides may be synthesised using methods well known in the art, some of which are described in more detail below.

By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Mézière et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

By “restriction to a MHC Class II molecule possessed by the patient can be demonstrated for the peptide” we mean that the peptide is able to bind to a particular MHC Class II possessed by the patient. That is not to say that a particular peptide cannot bind to another MHC Class II molecule. Peptides are generally only recognised in the context of a “self” MHC molecule, thus recognition of MHC-bound peptides by an individual's T cells is generally restricted by the MHC molecules expressed by the individual molecule.

Although binding to the given MHC Class II molecule may be demonstrated directly using suitable samples from the patient, whether or not a particular peptide can bind to a particular MHC Class II molecule (ie is restricted by a particular Class II molecule) can readily be determined in vitro using methods well known in the art, some of which are disclosed below.

Determination of the MHC Class II haplotype of the patient or the identification of particular MHC Class II genes possessed by the patient can readily be determined using any suitable method as is well known in the art, including the PCR-based methods described more fully below for example techniques based on those of Olerup & Zetterquist (1992) Tissue Antigens 29:225-235. Determination of the MHC Class II haplotype indicates which MHC molecules are expressible by an individual.

By “late phase response” we include the meaning as set forth in Allergy and Allergic Diseases (1997) A. B. Kay (Ed.), Blackwell Science, pp 1113-1130. The late phase response may be any late phase response (LPR). Preferably, the peptide is able to induce a late asthmatic response (LAR) or a late rhinitic response, or a late phase skin response or a late phase ocular response. Whether or not a particular peptide can give rise to a LPR can be determined using methods well known in the art; a particularly preferred method is that described in Cromwell O, Durham S R, Shaw R J, Mackay J and Kay A B. Provocation tests and measurements of mediators from mast cells and basophils in asthma and allergic rhinitis. In: Handbook of Experimental Immunology (4) Chapter 127, Editor: Weir D M, Blackwell Scientific Publications, 1986. Not all individuals who possess the particular MHC Class II molecule would experience a LPR following the administration of allergen or allergen-derived peptides since generation of the LPR is dependent upon prior allergic sensitisation to the allergen in question.

Thus, preferably, the peptide is able to induce a LPR in an individual who possesses the said MHC Class II molecule and who has been sensitised to the allergen in question. Whether or not an individual has been sensitised to the allergen in question may be determined by well known procedures such as skin prick testing with solutions of allergen extracts, induction of cutaneous LPRs, clinical history, allergen challenge and radio-allergosorbent test (RAST) for measurement of allergen specific IgE.

Preferably, the peptide is included in a composition containing a plurality of peptides derived from the said allergen. The peptides in the composition may or may not be multiple overlapping peptides (MOPs) derived from the polypeptide allergen. The plurality of peptides may be derived from the whole of the polypeptide allergen and therefore the peptides span the whole of the polypeptide chain or chains of the allergen.

However, they may be derived from only portions of the polypeptide allergen such that some portions of the polypeptide allergen are not represented in the plurality of peptides (for example, as is shown below, some peptides derived from an allergen may not be very soluble in aqueous solution and so may not be useful and other peptides may not show restriction to MHC Class II molecules). MOPs or any peptides derived from the allergen and present in the composition can be designed by reference to the amino acid sequence of the polypeptide allergen. Typically, the peptides are at least seven amino acid residues. Typically, the peptides would be between around 14 to 18 amino acid residues in length. It is preferred that the peptides have a reduced ability to bind IgE compared to longer peptides containing the same sequence. It is particularly preferred if the peptides are substantially incapable of binding IgE. Typically, when the MOPs overlap, the overlap is around one amino acid residue. This is particularly useful when the MOPs are used in in vitro T cell assays in order to identify MHC-binding peptides which may then be screened for their ability to induce LPR in an individual. More details of screening procedures are given below.

MHC Class II molecules are encoded by MHC Class II genes. There are at least three loci (DR, DQ and DP) that encode MHC Class II molecules, and each individual has two copies of each locus. These loci exhibit considerable genetic diversity and the preponderance of different MHC Class II genes (alleles) varies. The approximate frequencies of various MHC Class II genes (alleles) from a normal (disease free) population of people in England is described in Haworth S, Sinnott P, Davidson J & Dyer P. Caucasian England Normal In: HLA Typing 1997, Eds: Terasaki, P I and Gjertson, D W, Publishers: UCLA tissue typing laboratory, incorporated herein by reference.

For DR molecules, the most common in the Caucasian population are those that can be classified DR1, DR2, DR3, DR4, DR5, DR6, DR7, DR51, DR52 and DR53.

For DP molecules, the most common are DPB1*0201, DPB1*0301 and DPB1 *0401.

For DQ molecules, the most common are DQB1*0201, DQB1*0301, DQB1*0501, DQB1*0601 and DQB1*0602.

It is particularly preferred if the plurality of polypeptides administered to the patient includes peptides for which restriction to MHC Class II molecules can be demonstrated. It is particularly preferred if the plurality of peptides administered to the patient includes peptides for which restriction to the MHC Class II DR molecules DR2, DR3, DR4, and DR7 can be demonstrated. In a further embodiment it is preferred if the plurality of peptides further includes peptides for which restriction to any one or more of the MHC Class II DR molecules DR1, DR5 and DR6 can be demonstrated.

It is also particularly preferred if the plurality of peptides administered to the patient includes peptides for which restriction to the MHC Class II DR molecules DR51, DR52 and DR53 has been demonstrated.

It is also particularly preferred if the plurality of peptides administered to the patient includes peptides for which restriction to the MHC Class II DP molecules DPB1*0201, DPB1*0301 and DPB1*0401 can be demonstrated.

It is also particularly preferred if the plurality of peptides administered to the patient includes peptides for which restriction to the MHC Class II DQ molecules DQB1*0301 and DQB1*0601 can be demonstrated. In a further embodiment it is preferred if the plurality of peptides further includes peptides for which restriction to any one or more of the MHC Class II DQ molecules DQB1*0201, DQB1*0501 and DQB1*0602 can be demonstrated.

It is preferred if the plurality of peptides includes only a single peptide for which restriction to a particular MHC Class II molecule can be demonstrated.

Restriction to a particular Class II molecule can be demonstrated as has been described above and is described in more detail below. It will be appreciated that it may not be possible to derive a peptide for which restriction to a particular Class II molecule can be demonstrated; for example, a particular polypeptide allergen may not contain a T cell epitope which can be presented by every MHC Class II molecule. In this case, of course, such a peptide is not present in the plurality of peptides derived from the polypeptide allergen.

By “desensitising a patient to a polypeptide allergen” is meant inhibition or dampening of allergic tissue reactions induced by allergens in appropriately sensitised individuals. It will be appreciated that whether or not a patient is sensitive to a particular polypeptide allergen can be assessed using well known procedures such as skin prick testing with solutions of allergen extracts, induction of cutaneous LPRs, clinical history, allergen challenge and radio-allergosorbent test (RAST) for measurement of allergen specific IgE, and whether or not a particular patient is one who is expected to benefit from treatment may be determined by the physician based, for example, on such tests.

Administration of the peptide (such as the composition containing a plurality of peptides) may be by any suitable method, some of which are described below in more detail. Suitable amounts of the peptide may be determined empirically, but typically are in the range given below. As is described in a further aspect of the invention below, the invention also includes a method of determining an initial dose of peptide which is suitable to administer to the patient. A single administration of the peptide may be sufficient to have a beneficial effect for the patient, but it will be appreciated that it may be beneficial if the peptide is administered more than once, in which case typical administration regimes may be, for example, once or twice a week for 2-4 weeks every 6 months, or once a day for a week every four to six months.

A second aspect of the invention provides a composition comprising a plurality of peptides derived from a polypeptide allergen wherein for at least one of the peptides in the composition restriction to a MHC Class II molecule can be demonstrated and the composition is able to induce a late phase response in an individual possessing the given MHC Class II molecule. Preferably, at least one peptide is present in the composition for which restriction to each of MHC Class II DR molecules DR2, DR3, DR4 and DR7 can be demonstrated, provided of course that such peptides can be derived from the allergen.

Also preferably the composition may include peptides for which restriction to any one or more of the MHC Class II DR molecules DR1, DR5 and DR6 can be demonstrated.

Preferably, at least one peptide is present in the composition for which restriction to each of MHC Class II DR molecules DR51, DR52 and DR53 has been demonstrated.

Preferably, at least one peptide is present in the composition for which restriction to each of MHC Class II DP molecules DPB1*0201, DPB1*0301, and DPB1*0401 can be demonstrated.

Preferably, at least one peptide is present in the composition for which restriction to each of MHC Class DQ molecules DQB1*0301 and DQB 1*0601 can be demonstrated. In a further embodiment it is preferred if the plurality of peptides further includes peptides for which restriction to any one or more of the MHC Class II DQ molecules DQB1*0201, DQB1*0501 and DQB1*0602 can be demonstrated.

These preferences are all with the proviso that for any particular allergen it may not be possible to derive a peptide for which restriction to a particular Class II molecule can be demonstrated.

Although the composition (or a peptide within the composition) is able to induce a LPR in an individual possessing the given MHC Class II molecule (and as described below in more detail suitable compositions and peptides may be identified by their ability to induce a LPR), it should be appreciated that when the composition (or a peptide within the composition) is used to treat a patient it is preferable that a sufficiently low concentration of the composition or peptide is used such that no observable LPR will occur but the response will be sufficient to partially desensitise the T cells such that the next (preferably higher) dose may be given, and so on. In this way the dose is built up to give fill desensitisation but often without ever inducing a LPR in the patient (although, of course, the composition or peptide is able to do so at a higher concentration than is administered. It will be appreciated further, and as discussed in more detail below, induction of LPR in an individual is particularly useful in selecting appropriate compositions and peptides but is not essential in the clinical efficacy and treatment stages.

It will be appreciated that the composition may contain as many or as few peptides derived from the polypeptide allergen as will make it useful. Although in one embodiment of the method of desensitising the patient of the first aspect of the invention a single peptide may be administered to the patient wherein the peptide demonstrates restriction to a MHC Class II molecule possessed by the patient and the peptide is able to induce a late phase response in an individual who possesses the said MHC Class II molecule, it is preferred if the composition of the second aspect of the invention contains sufficient number of peptides, each of which demonstrate restriction to a particular MHC Class II molecule and which are able to induce a late phase response in an individual who possesses the said MHC Class II molecule, such that for at least 75% of the population a peptide is present in the composition which is MHC Class II restricted and which is capable of inducing a late phase response in an individual with an appropriate restricted MHC Class II molecule. More preferably the composition contains sufficient peptides such that for at least 80% of the population (and still more preferably at least 85%, or yet still more preferably 90% of the population) a peptide is present in the composition which is MHC Class II restricted and which is capable of inducing a late phase response in an individual with an appropriate restricted MHC Class II molecule.

In a particularly preferred embodiment, the composition contains (as the only polypeptide allergen-derived peptide components of the composition) peptides which are MHC Class II restricted and which are capable of inducing a LPR in an individual who possesses the given MHC Class II molecule. Preferably, the composition contains as the only polypeptide allergen-derived peptide components a sufficient number of peptides, each of which demonstrate restriction to a particular MHC Class II molecule and which are able to induce a LPR in an individual who possesses the said MHC Class II molecule, such that for at least 75% of the population a peptide is present in the composition which is MHC Class II restricted and which is capable of inducing a LPR in an individual with an appropriate restricted MHC Class II molecule.

It is well known that the frequency of particular MHC Class II molecules in a population varies with ethnic groups, and that for at least some ethnic groups the frequency of particular MHC Class II molecules is known (see, for example, HLA Typing 1997, supra). For example, the frequency of particular MHC Class II molecules is different in the Caucasian population compared to the Mongoloid population or Negroid population and so on. It will readily be appreciated that the polypeptide allergen-derived peptides to be included in a composition of tie invention may be selected according to the ethnic group to which the patient belongs. For example, compositions of the invention may readily be prepared for desensitisation to a particular polypeptide allergen by reference to the MHC Class II gene frequencies in the Caucasian or Mongoloid or Negroid populations.

A third aspect of the invention provides a composition of the second aspect of the invention packaged and presented for use in medicine. In particular, the composition will be packaged and presented with an indication of who may be treated (in particular who may benefit from being treated) with the composition including, if desirable, an indication of the MHC Class II molecules to which the peptides within the composition are restricted.

It will be appreciated that the composition of the second aspect of the invention is conveniently administered to the patient according to the method of the first aspect of the invention.

A fourth aspect of the invention provides a pharmaceutical formulation comprising a composition according to the second aspect of the invention and a pharmaceutically acceptable carrier. Suitable ingredients for pharmaceutical formulations are described in more detail below.

A fifth aspect of the invention provides the use of a peptide derived from a polypeptide allergen wherein restriction to a MHC Class II molecule possessed by a patient can be demonstrated for the peptide and the peptide is able to induce a late phase response in an individual who possesses the said MHC Class II molecule in the manufacture of a medicament for desensitising a patient to said polypeptide allergen.

A sixth aspect of the invention provides the use of a composition according to the second aspect of the invention in the manufacture of a medicament for desensitising a patient to said polypeptide allergen.

It will be appreciated that with respect to the method of the first aspect of the invention it may be desirable to determine which MHC Class II molecules the patient possesses in order to select an appropriate peptide or composition to administer to the patient. (It will be appreciated that this may be determined by determining the MHC haplotype of the individual by genetic means.) This is particularly desirable when the administration of a single peptide is contemplated. However, it will also be appreciated that when a composition is used which contains sufficient number of peptides, each of which demonstrate restriction to a particular MHC Class II molecule and which are able to induce a late phase response in an individual who possesses the said MHC Class II molecule, such that for at least 75% (or more preferably 80%, or 85% or 90%) of the population a peptide is present in the composition which is MHC Class II restricted and which is capable of inducing a late phase response in an individual with an appropriate restricted MHC Class II molecule, then it may not be necessary or desirable to type the patient to determine which MHC Class II molecules he or she possesses.

The polypeptide allergen may be any polypeptide allergen, some of which are described in more detail below.

A seventh aspect of the invention provides a method of selecting a peptide for use as an immunotherapeutic agent for desensitising a patient to a polypeptide allergen capable of eliciting an allergic response in the patient, which patient possesses a particular MHC Class II molecule, the method comprising the steps of (1) selecting a candidate peptide derived from the polypeptide allergen, (2) determining whether the candidate peptide demonstrates restriction to the said MHC Class II molecule, and (3) determining whether the candidate peptide is able to induce a late phase response in an individual who possesses the said MHC Class II molecule.

The candidate peptide may be any peptide derived from the polypeptide allergen and is, conveniently, a polypeptide in the size range described elsewhere as being a suitable size of a peptide for use in immunotherapy.

Whether or not the candidate demonstrates restriction to the said MHC Class II molecule may be determined by any suitable method such as those well known in the art, some of which are described in the Examples.

Whether or not the candidate peptide is able to induce a LPR can be determined by the methods described herein and which are well known in the art. It is particularly preferred if step (2) is carried out prior to step (3) and only candidate peptides which demonstrate restriction to the particular MHC Class II molecules are selected for testing in step (3).

It is particularly preferred that the individual in step (3) is an appropriately sensitised individual; that is to say an individual who has been sensitised previously to the allergen in question. It is those peptides which are capable of inducing a LPR and which demonstrate restriction to the particular MHC Class II molecule which are selected as an immunotherapeutic agent.

Determination of whether the candidate peptide demonstrates restriction to the said MHC Class II molecules may conveniently be done using a suitable T cell activation assay.

Thus, in one preferred embodiment the invention provides a method for selecting a peptide for use as an immunotherapeutic agent for desensitising a patient to an allergen capable of eliciting an allergic response in the patient which patient possesses a particular MHC Class II haplotype, comprising the steps of:

a) administering a candidate peptide to an individual who possesses the same said MHC Class II molecule as the patient and determining whether the peptide induces a late phase response; and

b) selecting a peptide capable of inducing a late-phase response as an immunotherapeutic agent.

The individual to whom the candidate peptide is administered for the purpose of determining whether the peptide induces a LPR may or may not be the patient.

In an eighth aspect, the invention provides a method for testing for candidate peptides for further selection according to the preferred embodiment discussed immediately above of the invention, comprising the steps of:

a) assaying a peptide or peptides in a T-cell activation assay and selecting peptides capable of inducing activation of an individual's T-cells;

b) tissue-typing the individual to determine MHC type;

c) determining the MHC molecule(s) bound by each candidate peptide; and

d) selecting a peptide or peptides satisfying part (a) above and capable of binding to an MHC type possessed by the individual, for use as a candidate peptide in a method according to the preferred embodiment discussed immediately above.

In a ninth aspect, the invention provides a method for selecting a peptide for use as an immunotherapeutic agent for desensitising a patient to an allergen comprising the steps of:

a) tissue-typing the patient to determine MHC Class II type; and

b) selecting, from a database of peptides which are known to bind to particular MHC molecules and induce a late phase response in an individual possessing such MHC Class II molecules, one or more peptides capable of binding to the MHC Class II molecules possessed by the individual.

Preferably, the individual is an appropriately sensitised individual who has been sensitised previously to the allergen in question.

In a tenth aspect, the invention provides a database of peptides characterised according to the seventh and eighth aspects of the invention.

TCRs are highly variable in their specificity. Variability is generated, as with antibody molecules, through gene recombination events within the cell. TCRs recognise antigen in the form of short peptides bound to molecules encoded by the genes of the Major Histocompatibility Complex (MHC). These gene products are the same molecules that give rise to “tissue types” used in transplantation and are also referred to as Human Leukocyte Antigen molecules (HLAs) which terms may be used interchangeably within this document. Individual MHC molecules possess peptide binding grooves which, due to their shape and charge are only capable of binding a limited group of peptides. The peptides bound by one MHC molecule may not necessarily be bound by other MHC molecules. As a result of this restricted peptide-MHC binding, T cell receptor recognition of a particular peptide is said to be “restricted” by the MHC molecule to which the peptide is bound. As used herein the term “allergen peptide-binding MHC” will be used to mean the MHC molecule(s) that bind the said allergen or allergen-derived peptide.

When a protein molecule such as an antigen or allergen is taken up by antigen presenting cells such as B lymphocytes, dendritic cells, monocytes and macrophages, the molecule is enzymatically degraded within the cell. The process of degradation gives rise to peptide fragments of the molecule which, if they are of the appropriate size, charge and shape, may then bind within the peptide binding groove of certain MHC molecules and be subsequently displayed upon the surface of antigen presenting cells. If the peptide/MHC complexes are present upon the antigen presenting cell surface in sufficient numbers they may then activate T cells which bear the appropriate peptide/MHC-specific T cell receptors.

Due to the polymorphic nature of the MHC, individuals in an outbred population such as man will express different combinations of MHC molecules on their cell surfaces. Since different MHC molecules can bind different peptides from the same molecule based on the size, charge and shape of the peptide, different individuals will display a different repertoire of peptides bound to their MHC molecules.

Identification of universal MHC-binding peptide epitopes in an outbred population such as man is more difficult than in inbred animals (such as certain strains of laboratory mice). On the basis of differential MHC expression between individuals and the inherent differences in peptide binding and presentation which this brings, it is unlikely that a single peptide can be identified which will be of use for desensitisation therapy in man for most diseases unless the association of a particular MHC molecule with that disease is very strong. For example, the HLA-B27 molecule has been shown to have a close relationship with ankylosing spondylitis, where approximately 90% of sufferers express HLA-B27. For some autoimmune diseases, certain disease HLA associations have been demonstrated eg HLA-DR4 and rheumatoid arthritis, but these associations are much weaker than for ankylosing spondylitis.

In allergic diseases, associations are even weaker if demonstrated at all. For this reason, it is unlikely that therapies centred around a single peptide (even an immunodominant one) or small numbers of peptides will be optimally effective as desensitisation therapies. The conclusion drawn in the art where MHC binding allergen epitopes have been identified is that even if an immunodominant epitope is identified, it would appear that it is required to react with a variety of restricted MHCs to be of therapeutic value (see Van Neerven R J J et al (1994) J Immunol 152, 4203-4210; Higgins J A et al (1994) J Allerg Clin Immunol 93, 891-899).

As set forth herein, it has now been observed that a patient may be desensitised to a particular allergen by the administration of a peptide or a composition containing a peptide that is able to bind to at least one MHC molecule of said patient and which is able to induce a LPR in an individual who possesses the same MHC Class II molecule type. According to the present invention, therefore, the concept of “universal” desensitising peptides is rejected in favour of a selective approach which takes into account tissue type. Nevertheless, it will be appreciated that using a composition containing a plurality of peptides according to the present invention may be “universal” in the sense that a single composition may be used for most of the population, but that this is still selective on the basis that the composition contains peptides which are restricted by a particular MHC Class II molecule.

It can be hypothesised that eosinophil-dependent mucosal tissue damage, including LPR, is under T-cell control. For example, by in situ hybridisation the numbers of mRNA positive cells for the Th2-type (IL-4 and IL-5) and eosinophil-active cytokines (IL-3, IL-5 and GM-CSF) were shown to be elevated in asthmatics both at baseline (Robinson et al (1992) N Engl J Med 326: 298-304) and following allergen-induced LAR (Bentley et al (1993) Am J Respir Cell Mol Biol 8:35-42). Furthermore IL-4 and IL-5 mRNA co-localised largely to CD4+ T cells (Ying et al (1997) J Immunol 158:3539-3544). A T cell component of the LAR is also suggested by the observation that cyclosporin A attenuated the LAR, but not the EAR, provoked by allergen inhalation (Sihra et al (1997) Thorax 52:447-452). Furthermore a single infusion of anti-CD4 produced significant improvement in lung function in chronic corticosteroid-dependent asthmatics. However it has been difficult to determine whether T cell activation, as an initiating event, leads directly to airway narrowing in asthmatic patients and therefore an asthmatic response.

As described herein, it has now been shown that T cells can be selectively activated, and then rendered unresponsive. Moreover the anergising or elimination of these T-cells leads to desensitisation of the patient for a particular allergen. The desensitisation manifests itself as a reduction in response to an allergen or allergen-derived peptide, or preferably an elimination of such a response, on second and further administrations of the allergen or allergen-derived peptide. The second administration may be made after a suitable period of time has elapsed to allow desensitisation to occur; this is preferably any period between one day and several weeks. An interval of around two weeks is preferred.

Based on these results, the invention provides a method for desensitising a patient to a polypeptide allergen which comprises the administration to the patient of a peptide specifically selected to induce LPR and subsequent desensitisation in the patient wherein the peptide is restricted by a particular MHC Class II molecule and capable of inducing LPR in an individual who possesses the given MHC Class II molecule to which the peptide is restricted. The peptides for desensitisation may be selected according to whether they induce LPR.

LPR is defined as set forth in Allergy and Allergic Diseases (1997) A. B. Kay (Ed.), Blackwell Science, pp 1113 to 1130, and includes asthmatic, cutaneous and nasal late phase responses as described above.

As noted above, the peptide which is administered may be included in a composition containing a plurality of peptides derived from the allergen.

Preferably, the peptides are derivatives of the allergen itself, and retain at least one common antigenic determinant of the allergen. “Common antigenic determinant” means that the derivative in question retains at least one antigenic function of the allergen. Antigenic functions include possession of an epitope or antigenic site that is capable of binding to TCRs which recognise the allergen or fragments thereof. Thus, the peptides provided by the present invention include splice variants encoded by mRNA generated by alternative splicing of a primary transcript encoding the allergen, amino acid mutants, glycosylation variants and other covalent derivatives of the allergen which retain at least an MHC-binding property of the allergen. Exemplary derivatives include molecules wherein the peptide of the invention is covalently modified by substitution, chemical, enzymatic, or other appropriate means with a moiety other than a naturally occurring amino acid. Further included are naturally occurring variants of the allergen found in a particular species. Such a variant may be encoded by a related gene of the same gene family, by an allelic variant of a particular gene, or represent an alternative splicing variant of the allergen gene.

Derivatives of the allergen also comprise mutants thereof, which may contain amino acid deletions, additions or substitutions, subject to the requirement to maintain at least one feature characteristic of the allergen. Thus, conservative amino acid substitutions may be made to peptides according to the invention substantially without altering the nature of the allergen, as may truncations from the N or C termini. Deletions and substitutions may moreover be made to the fragments of the allergen comprised by the invention. Peptides may be produced from a DNA which has been subjected to in vitro mutagenesis resulting eg in an addition, exchange and/or deletion of one or more amino acids. Preferably, peptides are produced by peptide synthesis according to known techniques using commercially available peptide synthesisers. Mutations and/or truncations may thus be made by changing the amino acid sequence during the synthesis procedure.

Suitable variants capable of binding to TCRs may be derived empirically or selected according to known criteria. Within a single peptide there are certain residues which contribute to binding within the MHC antigen binding groove and other residues which interact with hypervariable regions of the T cell receptor (Allen et al (1987) Nature 327:713-5). Within the residues contributing to T cell receptor interaction, a hierarchy has been demonstrated which pertains to dependency of T cell activation upon substitution of a given peptide residue. Using peptides which have had one or more T cell receptor contact residues substituted with a different amino acid, several groups have demonstrated profound effects upon the process of T cell activation. Evavold & Allen (1991) Nature 252:1308-10) demonstrated the dissociation of T cell proliferation and cytokine production. In this in vitro model, a T cell clone specific for residues 64-76 of haemoglobin (in the context of I-E^{k}), was challenged with a peptide analogue in which a conservative substitution of aspartic acid for glutamic acid had been made. This substitution did not significantly interfere with the capacity of the analogue to bind to I-E^{k}. Following in vitro challenge of a T cell clone with this analogue, no proliferation was detected although IL-4 secretion was maintained, as was the capacity of the clone to help B cell responses. In a subsequent study the same group demonstrated the separation of T cell-mediated cytolysis from cytokine production. In this instance, the former remained unaltered while the latter was impaired. The efficacy of altered peptide ligands in vivo was initially demonstrated in a murine model of EAE (experimental allergic encephalomyelitis) by McDevitt and colleagues (Smilek et al (1991) Proc Natl Acad Sci USA 88:9633-9637). In this model EAE is induced by immunisation with the encephalitogenic peptide Ac1-11 of MBP (myelin basic protein). Substitution at position four (lysine) with an alanine residue generated a peptide which bound well to its restricting element (Aα^{u}Aβ^{u}), but which was non-immunogenic in the susceptible PL/JxSJLF1 strain and which, furthermore prevented the onset of EAE when administered either before or after immunisation with the encephalitogenic peptide. Thus, residues can be identified in peptides which affect the ability of the peptides to induce various functions of T-cells.

Advantageously, peptides may be designed to favour T-cell proliferation and induction of desensitisation. Metzler and Wraith have demonstrated improved tolerogenic capacity of peptides in which substitutions increasing peptide-MHC affinity have been made (Metzler & Wraith (1993) Int Immunol 5:1159-65). The demonstration that an altered peptide ligand can cause long-term and profound anergy in cloned T cells (Sloan-Lancaster et al (1993) Nature 363:156-9) is particularly relevant to the applications of such peptide analogues in immunotherapy for diseases such as autoimmunity and allergy, in addition to the induction of host/donor-specific tolerance in transplantation.

Derivatives which retain common antigenic determinants are preferably fragments of the allergen. Fragments of the allergen comprise individual domains thereof, as well as smaller polypeptides derived from the domains. Preferably, smaller polypeptides derived from the allergen according to the invention define a single epitope of the allergen capable of binding a TCR. Fragments may in theory be almost any size, although smaller fragments are more likely to be restricted to a single MHC molecule and are thus preferred. Preferably, fragments will be between 5 and 50, preferably between 5 and 25, and advantageously about 17 amino acids in length. It is preferred if the peptides do not invoke an IgE response and do not lead to the release of histamine from enriched basophils or mast cell preparations from most sensitised individuals.

Candidate peptides potentially capable of inducing LPR in a patient may be preselected in order to maximise the chances of identifying a therapeutically useful peptide in in vivo tests. The steps of this aspect of the invention comprise the determination that the peptide is MHC Class II restricted, for example it is capable of causing T-cell proliferation when associated with an MHC molecule present in the patient to be treated. Thus, in a particular embodiment the selection procedure can be broken down into three steps, performed either sequentially (in any order) or together:

a) assaying a peptide or peptides in a T-cell activation assay and selecting peptides capable of inducing activation in an individual's T-cells;

b) tissue-typing the individual to determine MHC Class II type; and

c) determining the MHC Class II molecule bound by each candidate peptide.

Steps (a) and (c), in particular, may be combined in a single T-cell activation assay. Preferably, the assay involves the use of cells transfected to express a particular MHC molecule, and the binding of the peptide to this MHC assessed by its ability to induce T-cell proliferation in the presence of the transfected cells alone. Suitable transfected cells are readily available and can, in any case, be readily made by transfecting the cloned genes into suitable cell lines.

Preferably, a peptide selected according to the above procedure is tested for its ability to induce LPR in an individual. If LPR is induced, repeated administration will result in desensitisation to the allergen from which the peptide is derived.

However, once a peptide has been determined to bind a particular MHC Class II type and to be capable of inducing LPR when administered to an individual possessing that MHC Class II type, it can be used to induce desensitisation to the relevant allergen in substantially any patient possessing the required MHC Class II molecule. Therefore, peptides derived from particular allergens may be characterised according to their binding to particular MHC Class II types and their ability to induce LPR, thus providing a database from which a suitable peptide may be selected for any given patient upon tissue typing of that patient. Additionally or alternatively, a preparation containing a plurality of MHC-binding peptides capable of inducing LPR may be employed which will be effective in desensitising the majority of sensitised individuals.

Thus, in one embodiment antigen presenting cells may be isolated from a patient known to be sensitive to a particular allergen or allergens, and based on the peptide-binding MHC molecules displayed by said cells, a peptide may be selected for use in desensitising said patient by virtue of its ability to bind to at least one MHC molecule. The invention accordingly provides a method for selecting a peptide for use as an immunotherapeutic agent for desensitising a patient to an allergen comprising the steps of:

a) tissue-typing the patient to determine MHC Class II type; and

b) selecting, from a database of peptides which are known to bind to particular MHC Class II molecules and induce a late phase response in an individual possessing such MHC Class II molecules, one or more peptides capable of binding to the MHC Class II molecules possessed by the patient.

For the avoidance of doubt, the individual referred to in part (b) above need not necessarily be the same individual as the patient undergoing treatment whom is tissue typed in part (a). In fact, once the MHC Class II restriction of a particular allergen-derived peptide is determined, and it has been determined that the peptide is capable of inducing a LPR in an individual, particularly an appropriately sensitised individual, who possesses the said MHC Class II molecule, there is no requirement to test the ability of the patient's own MHC Class II molecules.

Allergens that may be amenable to desensitisation procedures as described herein include the peptides derived or chosen from the list comprising the allergens; Fel d 1 (the feline skin and salivary gland allergen of the domestic cat Felis domesticus—the amino acid sequence of which is disclosed in WO 91/06571), Der p I, Der p II, Der fI or Der fII (the major protein allergens from the house dust mite dermatophagoides—amino acid sequences disclosed in WO 94/24281).

The invention is applicable substantially to any allergen, including allergens present in any of the following: grass, tree and weed (including ragweed) pollens; fungi and moulds; foods eg fish, shellfish, crab lobster, peanuts, nuts, wheat gluten, eggs and milk; stinging insects eg bee, wasp and hornet and the chirnomidae (non-biting midges); spiders and mites, including the house dust mite; allergens found in the dander, urine, saliva, blood or other bodily fluid of mammals such as cat, dog, cows, pigs, sheep, horse, rabbit, rat, guinea pig, mouse and gerbil; airborne particulates in general; latex; and protein detergent additives.

Where the allergen is an insect protein, the peptides may be selected from the group comprising: housefly, fruit fly, sheep blow fly, screw worm fly, grain weevil, silkworm, honeybee, non-biting midge larvae, bee moth larvae, mealworm, cockroach and larvae of Tenibrio molitor beetle. All these being insect allergens, they are of particular relevance to allergic problems arising in the workplace.

Where the allergen is the Fel d 1 allergen, useful peptides may preferably comprise a sequence as shown in any one of SEQ ID Nos. 1 to 3. Particular preferred peptides for use in the methods of the invention are those with the sequence given in SEQ ID Nos. 1 or 2 or 3. Preferred compositions of the invention include those that contain the peptides with the sequence given in SEQ ID Nos. 1, 2 and 3, and compositions containing the MHC Class II-restricted peptides of the thirteen peptides described in Example 7 and for which can be determined a LPR in an individual possessing appropriate MHC Class II molecules.

A database according to the invention includes information on the MHC Class II molecule(s) bound by peptides in the database and the ability of the peptides to induce a LPR in patients possessing such MHC Class II molecule(s). Thus, the database allows a practitioner to select peptides capable or potentially capable of eliciting a LPR and therefore desensitisation in a particular patient on the basis of that patient's tissue type.

The invention moreover provides a peptide listed in a database according to the invention, for use in therapy. Preferably, such peptides are useful in methods for desensitising patients to allergens in accordance with the methods set forth herein. Peptides to be included in the database, and peptides which may be useful either individually or as a mixture in a composition of the invention may readily be selected by the methods of the invention from polypeptide allergens whose polypeptide sequences, or reference to polypeptide sequences, are given in Example 6.

The MHC molecules expressed on APCs which bind peptides derived from a specific allergen may be identified by methods known in the art, such as T cell proliferation studies with MHC blocking antibodies, and PCR techniques, for example techniques based on those of Olerup & Zetterquist (1992) Tissue Antigens 29:225-235. Thus, antigen-presenting cells, expressing a variety of MHC molecules may be incubated with allergen and T cells and the latter observed for proliferation. Addition of antibodies to specific MHC classes may then be made in repeat incubations in order to identify the restricted MHC in respect of the allergen being tested. See Van Neerven R J J et al (1994) Immunol 82:351-356, and Yssel H et al (1992) J Immunol 148:738-745.

Alternatively, cells presenting a single MHC Class II type, for example cells such as fibroblast cells transfected with the genes encoding an MHC Class II molecule, may be incubated with individual peptides for which T-cell clones or lines are known to be specific. Culturing of such T-cell clones or lines with peptide presented by the appropriate MHC Class II molecule will lead to T-cell proliferation. T cell proliferation is not the only indicator that a particular peptide binds to a particular MHC Class II molecule on an APC. Other indicators include the secretion of measurable soluble products such as cytokines, changes in intracellular calcium levels, and other means of measuring T cell activation which are well known in the art.

Preferred fibroblasts for use in this aspect of the invention include human or murine fibroblasts, particularly L-cells.

The latter method may be used in a combinatorial approach, in which groups of peptides may be tested together and effective peptides identified by standard combinatorial techniques.

Specific epitopes of the allergen or peptide derived therefrom that bind to at least one MHC Class II molecule may then be identified by standard procedures and used in desensitisation procedures as described herein. Accordingly, the invention provides peptides when selected according to the foregoing aspects of the invention.

For example, when the allergen is a cat allergen such as the Fel d 1 protein, then the MHC molecule may include DR13 or DR1 class II MHC, and a peptide that binds to DR13 and/or DR1 or any of its sub-types that may be used in a desensitisation procedure is that shown in SEQ. ID No. 3.

The peptides identified in such a manner, and those of use in the methods of the present invention may be used in desensitisation procedures that typically involve sequential administration of said peptide. Although the first administration of the peptide may induce a measurable or observable LPR, as has been described elsewhere the peptide or composition administered to the patient may be at a concentration that does not invoke a measurable or observable LPR. Subsequent administration will lead to desensitisation of the patient. For example, if the peptide is that of SEQ. ID No. 3 (a fragment of the Fel d 1 allergen), then upon first administration of this peptide a LPR will be observed. Subsequent administration of this peptide results in a weaker reaction or no reaction, the patient having been desensitised.

The invention also relates to the use of a peptide in desensitising a patient against an allergen, the peptide being identified by its capability to bind to at least one MHC Class II molecule present in an individual and induce LPR in an individual who possesses the said MHC Class II molecule, wherein the patient also possesses the given MHC Class II molecule.

Peptides may be administered to a patient singly or in combination (for example as a composition as defined above). Thus, the database according to the invention may be used to prepare a designer vaccine which may be used to desensitise a patient to a chosen allergen, on the basis of the patient's MHC Class II type. The MHC Class II type can be correlated to the known MHC Class II binding characteristics of the peptides listed in the database, and the appropriate peptides selected and combined to form a designer vaccine. Similarly, the database may be used to design compositions (ie mixtures of peptides) which contain sufficient number of peptides, each of which demonstrate restriction to a particular MHC Class II molecule and which are able to induce a late phase response in an individual who possesses the said MHC Class II molecule, such that for at least 75% (preferably at least 80% or 85% or 90%) of the population a peptide is present in the composition which is MHC Class II restricted and which is capable of inducing a late phase response in an individual with the appropriate MHC Class II molecule.

Whilst it may be possible to design a vaccine which targets all or most of the epitopes on a particular antigen, this is unnecessary due to linked suppression of T-cells. Linked suppression is a phenomenon in which administration of a single epitope from a protein leads to the induction of a population of regulatory peptide-specific T lymphocytes which, by release of soluble factors such as TGFβ and/or IL-10, are able to suppress or modify responses of non-tolerant T cells specific for other epitopes within the same protein and in some models epitopes derived from other proteins (“bystander suppression”) (Davies et al (1996) J Immunol 156:3602-7). In transplantation models, such regulatory T cells have been demonstrated to be capable of inducing a similar phenotype in naive T cells. This has given rise to the term “infectious tolerance” (Qin et al (1993) Science 259:974-7) which may be a mechanism for effecting long-term hyporesponsiveness.

Linked suppression is thought to occur when peptide-specific regulatory T cells engage peptide/MHC complexes on the surface of the same or neighbouring APC as T cells specific for other epitopes. The latter may be responding to epitopes derived from the same molecule as the regulatory T cells or from a distinct molecule being processed by the same APC. This phenomenon allows desensitisation of patients to one or multiple allergens by the administration of a limited number of peptides.

Whilst it may be possible for the peptides or compositions according to the invention to be presented in raw form, it is preferable to present them as a pharmaceutical formulation. Thus, according to a further aspect, the present invention provides a pharmaceutical formulation comprising a peptide or composition according to the invention together with one or more pharmaceutically acceptable carriers therefor and optionally one or more other therapeutic ingredients. The carrier(s) must be ‘acceptable’ in the sense of being compatible with the other ingredients of the formulation and not deleterious to the recipient thereof. Typically, carriers for injection, and the final formulation, are sterile and pyrogen free.

The formulations include those suitable for oral (particularly inhaled), parenteral (including subcutaneous, transdermal, intradermal, intramuscular and intravenous and rectal) administration, although the most suitable route may depend upon for example the condition and disorder of the recipient. The formulations may conveniently be presented in unit dosage form and may be prepared by any of the methods well known in the art of pharmacy. All methods include the step of bringing into association a compound of the present invention as herein defined or a pharmacologically acceptable salt or solvate thereof (“active ingredient”) with the carrier which constitutes one or more accessory ingredients.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Formulations for inhalation may be presented in any of the ways known to be effective eg metered dose inhalers.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Preferred unit dosage formulations are those containing an effective dose, as hereinbelow recited, or an appropriate fraction thereof, of the active ingredient.

The compounds of the invention may typically be administered intranasally, by inhalation, orally or via injection at a dose of from 0.0001 to 1 μg/kg per dose. Preferred are doses in the region of 10 to 150 μg per human patient, advantageously about 80 μg.

A further aspect of the invention provides a method of determining an initial dose of an immunotherapeutic peptide for desensitising a patient to a polypeptide allergen, which peptide is derived from the allergen and wherein restriction to a MHC Class II molecule possessed by the patient can be demonstrated for the peptide and the peptide is able to induce a late phase response in an individual who possesses the said MHC molecule, the method comprising (1) determining the dose which is able to generate an observable late phase response in a given proportion of individuals who possess the said MHC molecule and in whom the peptide is able to induce a late phase response and (2) selecting a lower dose which is incapable of inducing an observable late phase response in substantially all individuals who possess the said MHC molecule and in whom the peptide is able to induce a late phase response.

Preferably, the individuals who possess the said MHC molecules are appropriately sensitised; that is to say that the individuals have been sensitised previously to the allergen in question.

The initial dose which is administered to the patient to be desensitised is, as is described above, one which may not itself give rise to an observable LPR.

In step (1) of the method of determining an initial dose the given proportion of individuals may be any suitable proportion of, but not all, individuals as given. Typically, the proportion is 50% of individuals as given, but it may be, for example, 30% or 40% or 60% or 70% of individuals as given. In step (2), the lower dose may be the maximum dose that is incapable of inducing an observable late phase response in substantially all individuals who possess the said MHC molecules and in whom the peptide is able to induce a LPR.

Typically, but it will be appreciated that this will vary from peptide to peptide, the lower dose is between 10-fold and 100-fold lower than the dose which induces an observable LPR in 50% of suitable individuals (a suitable individual is one who is appropriately sensitised and has the appropriate MHC Class II molecule(s) to facilitate peptide reactivity.

The LPR may be any suitable LPR as herein disclosed. Suitably, late asthmatic reactions are determined in asthmatics, late nasal reactions in rhinitics and late phase skin reactions in all allergic individuals.

It is preferred if the LPR is a late cutaneous reaction.

The methods of the invention are particularly suited for use in connection with human patients. However, it will be appreciated that animals, particularly mammals, and more particularly domestic and farm animals such as dogs and cats, may suffer from allergies due to polypeptide allergens. The methods of the invention include methods in connection with such animals. Although the specification refers to MHC and HLA Class II molecules, equivalent molecules exist in mammals other than humans as is well known in the art.

The invention is further described, for the purpose of illustration only, in the following examples, which refer to the figures.

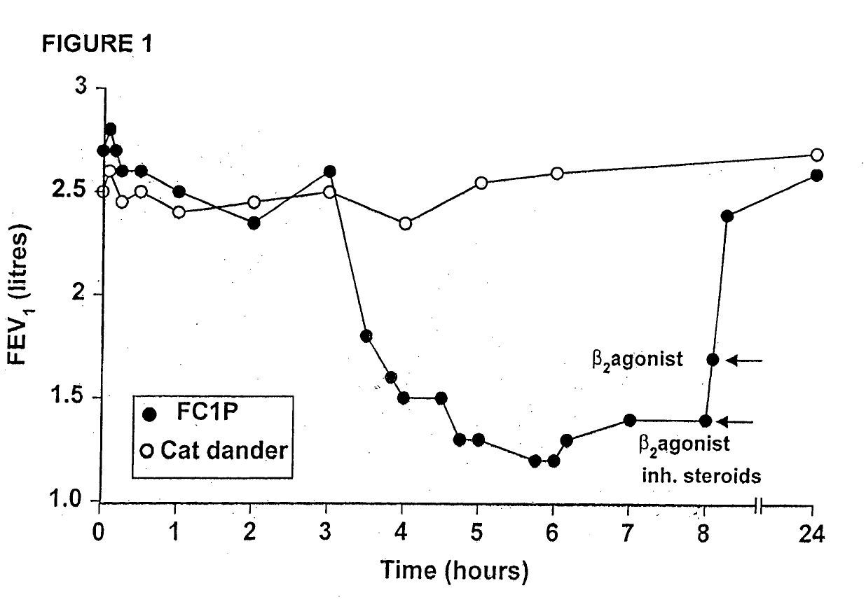

FIG. 1. The three peptides comprising FCIP (solid circles; 80 μg) or vehicle control (open circles) are injected intradermally at time zero on two separate days. Forced expiratory volume in 1 second (FEV1) is measured at intervals as a readout of lung function over a 24 hr period. The use of rescue medication is indicated by arrows.

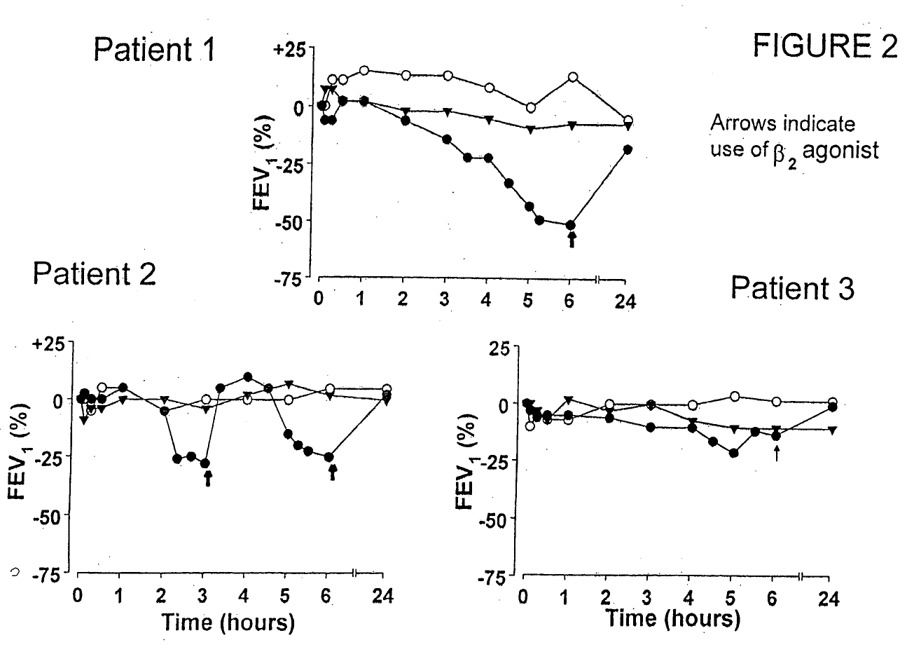

FIG. 2. Repeated administration of FC1P leads to a reduced lung response. Three patient volunteers who develop a late asthmatic reaction following administration of FC1P (closed circles), are challenged again with the same dose after a period of at least 2 weeks. No significant fall in FEV1 is observed following the second challenge (closed triangles). Open circles indicate the control day. Arrows indicate the use of bronchodilators.

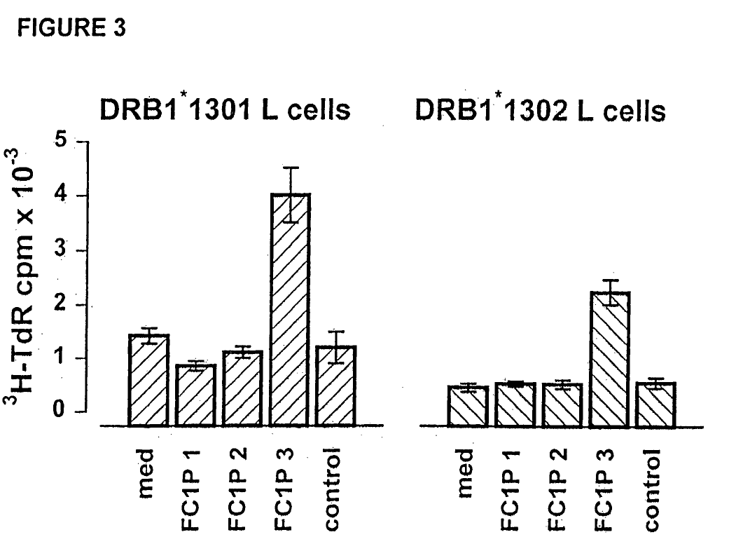

FIG. 3. Murine L cells expressing two DR13 variants, DRB1*1301 and 1302 are incubated overnight with each of the three FC1P peptides, or a control peptide or medium alone. Cells are washed and incubated for one hour with a cytostatic agent to prevent proliferation in the subsequent assay. L cells are then incubated for 48 hours with T cells from a T cell line raised to whole cat dander (and including the Fel d 1 protein). Proliferation of the T cells is measured by their incorporation of the radiolabelled compound ³H-thymidine. T cells demonstrate a statistically significant response to the DR13 L cells and peptide FC1P3 (KALPVVLENARILNCV) but not to the other peptides/control.

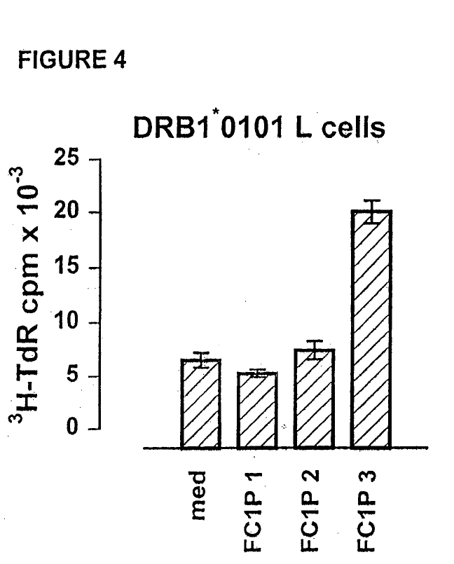

FIG. 4. Human fibroblasts expressing the DR1 allele DRB1*0101 are incubated overnight with each of the three FC1P peptides, or medium alone, as described for FIG. 3. In T cell proliferation assays, T cells demonstrate a statistically significant response to the DR1 expressing cells and peptide FC1P3 (KALPVVLENARILNCV) but not to the other peptides/control.

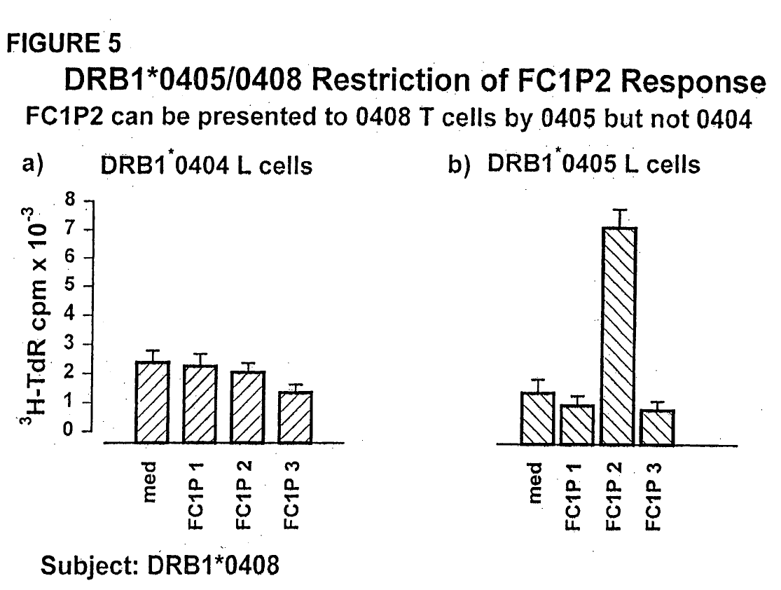

FIG. 5. Human fibroblasts expressing the DR4 alleles DRB1*0404 and DRB1*0405 are incubated overnight with each of the three FC1P peptides, or medium alone, as described for FIG. 3. FIG. 5 a) and b): in T cell proliferation assays, DRB1*0408 responder cells demonstrate a statistically significant response to the DRB1*0405 expressing cells and peptide FC1P2 (EQVAQYKALPVVLENA) but not to DRB1*0404 expressing cells and peptide FC1P2 or to the other peptides/control.

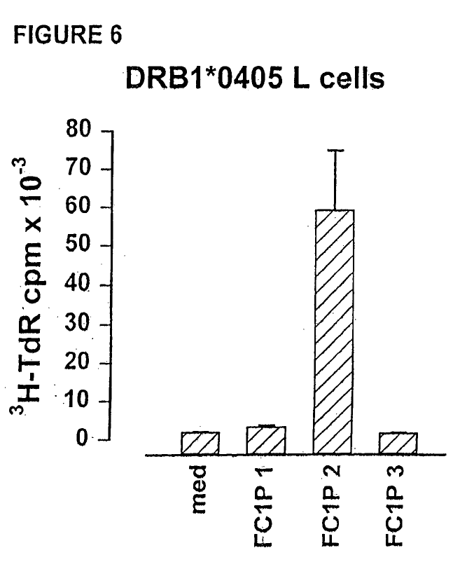

FIG. 6. Human fibroblasts expressing the DR4 allele DRB1*0405 are incubated overnight with each of the three FC1P peptides, or medium alone, as described for FIG. 3. In T cell proliferation assays, DRB1*0405 responder cells demonstrate a statistically significant response to the DRB1*0405 expressing cells and peptide FC1P2 (EQVAQYKALPVVLENA) but not to the other peptides/control.

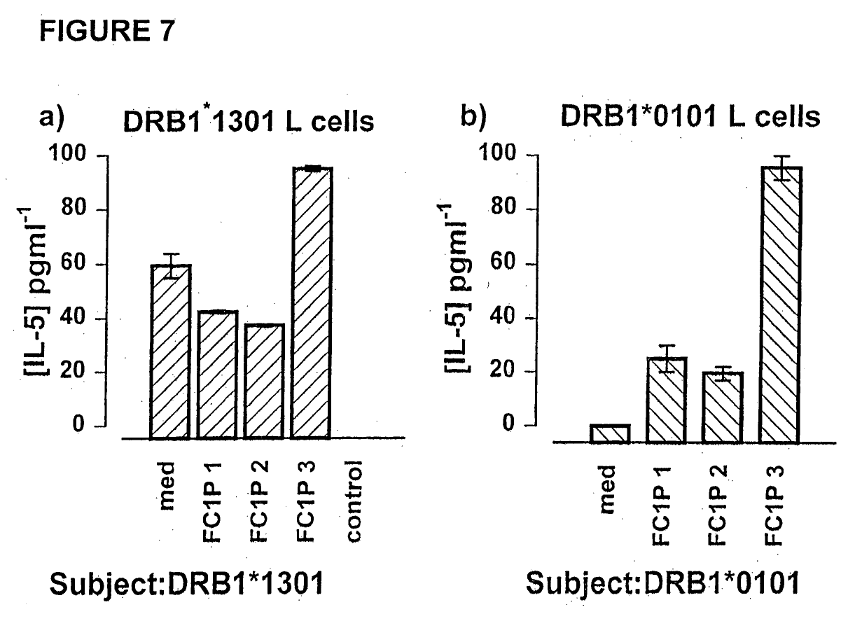

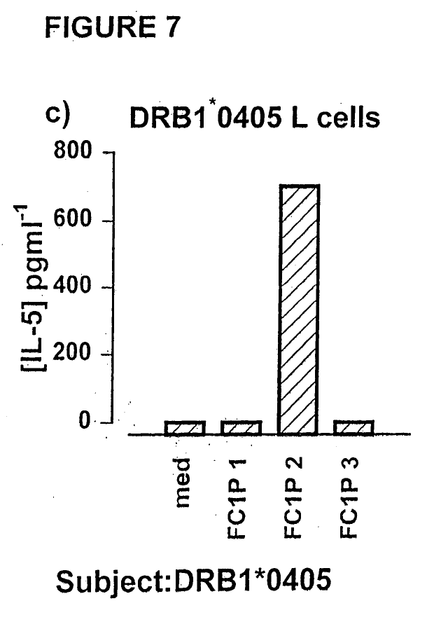

FIG. 7. The T cell proliferation responses observed in FIGS. 3, 4 and 6 are confirmed by [IL-5] measurement in FIGS. 7(a), 7(b) and 7(c) respectively. As expected, these results show that IL-5 production correlates with T-cell proliferation.

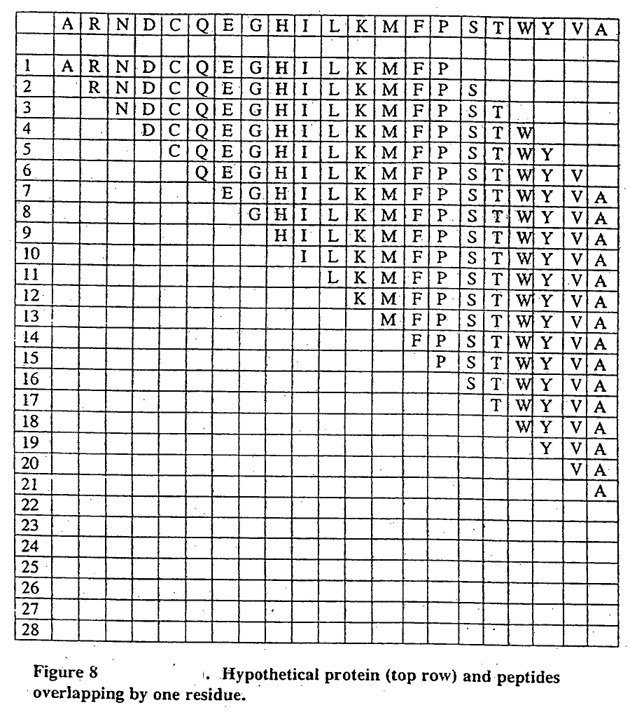

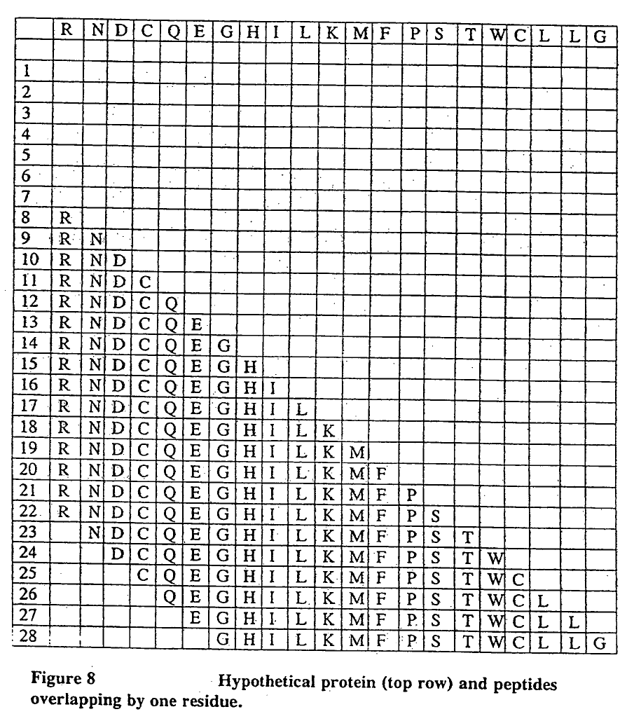

FIG. 8. Hypothetical protein and peptides (15mers) derived from overlapping by one residue.

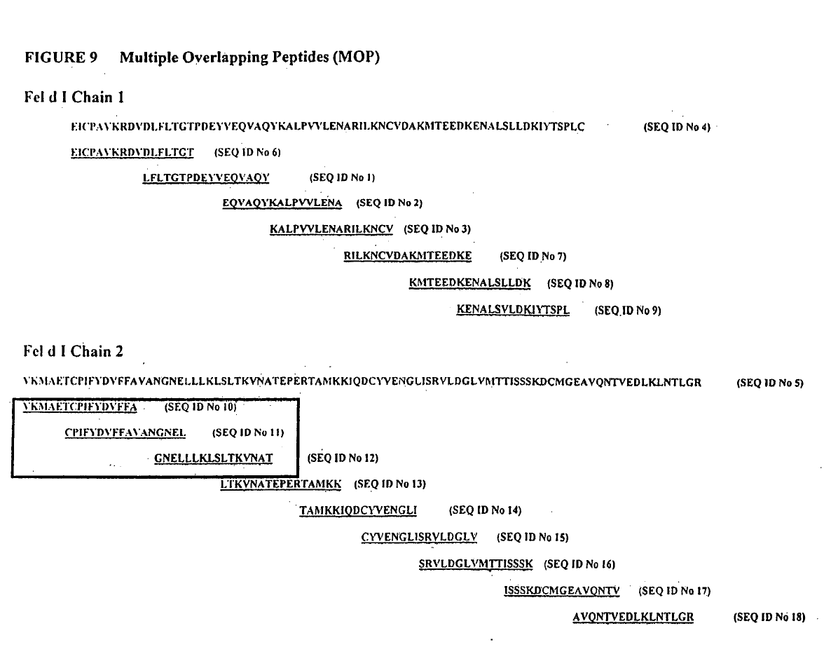

FIG. 9. Multiple overlapping peptides (MOP) from die cat allergen Fel d I. The three sequences within the box were insoluble in aqueous solution and as a result were excluded from the MOP preparation for clinical use.

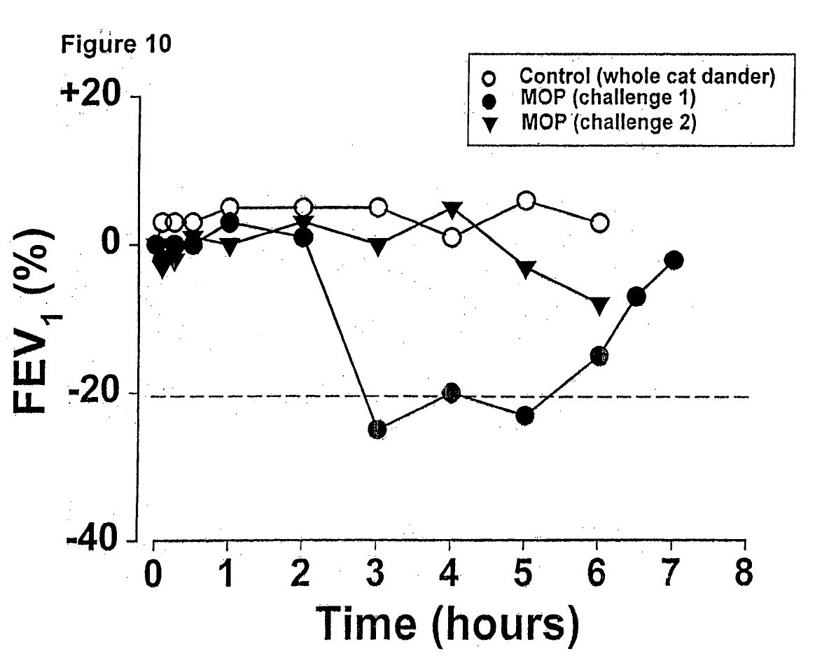

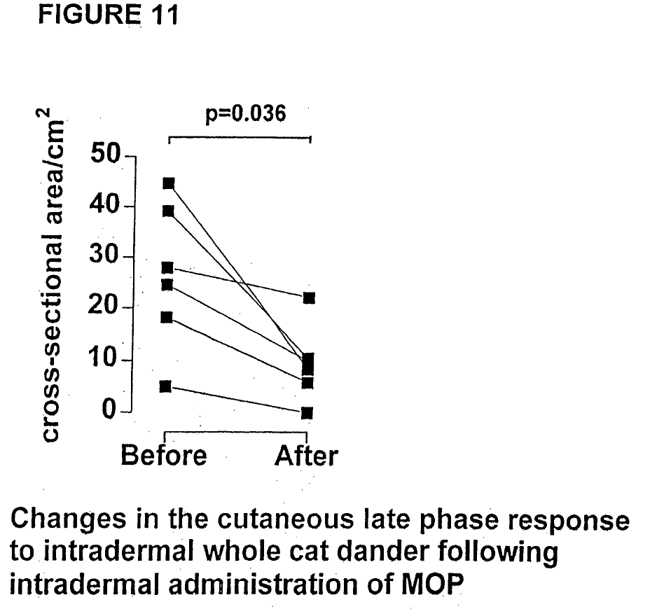

FIG. 10. An example of a LAR induced by the Fel d I MOP. The intradermal administration of 13 peptides which comprise MOP (solid circles; 2.5 μg, day 1) induce a fall in FEV1 of greater than 20% at 3 hours. Control day administration of 30BU cat dander extract does not induce a fall in FEV1 (open circles). A second administration of MOP (solid triangles; 2.5 μg, day 66) results in an attenuated fall in FEV1 which does not reach 20%. Arrows indicate the use of rescue medication (β₂ agonists).

FIG. 11. Changes in the cutaneous late phase response to whole allergen 6 hours after intradermal administration of whole cat dander extract before and after intradermal administration of MOP.

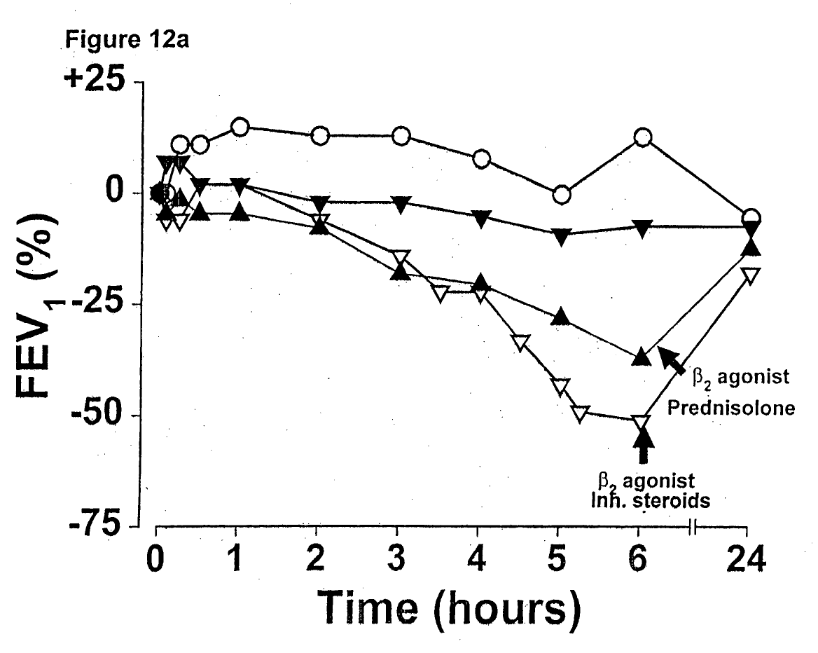

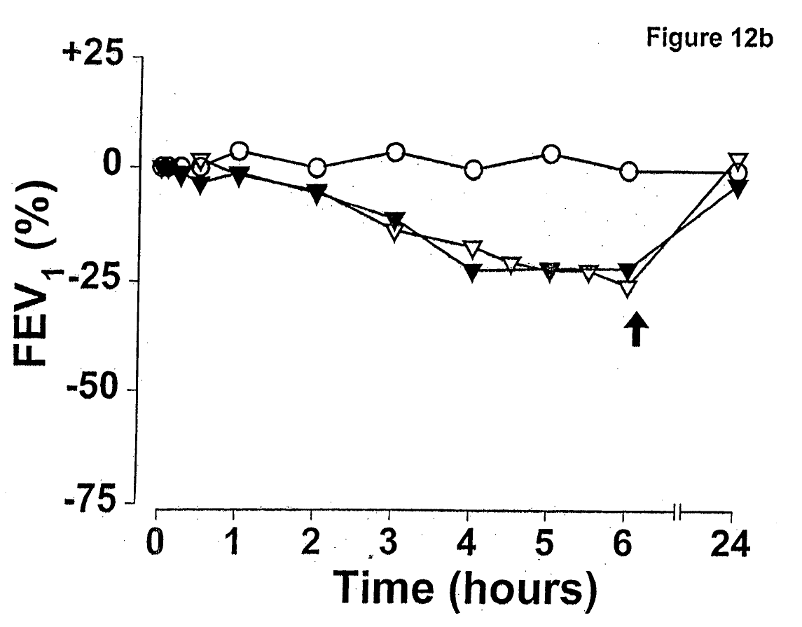

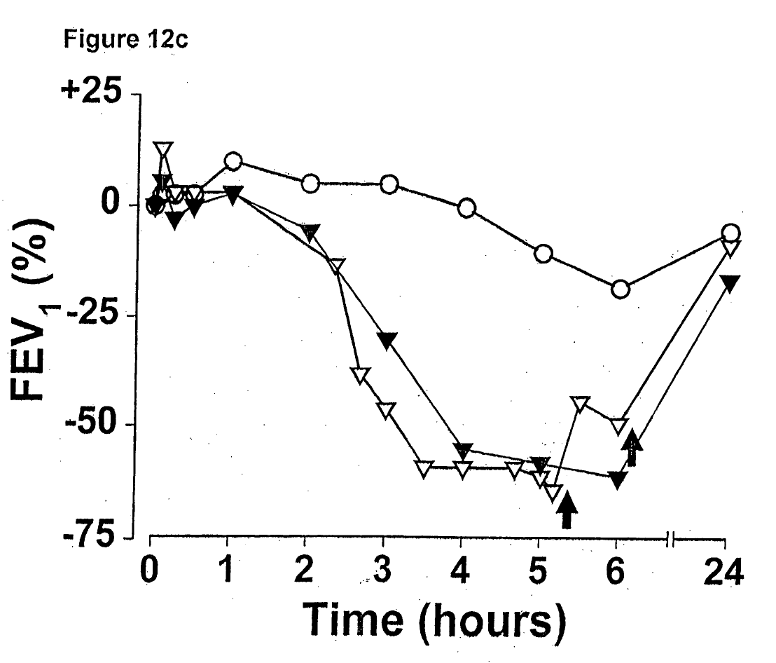

FIG. 12. The 3 peptides comprising FC1P (open down triangles: 80 μg, Figures (a), (b) and (c)) were administered intradermally to cat allergic asthmatic subjects inducing a fall in FEV1 of greater than 20% compared to a control day (open circles; 30BU whole cat dander extract, FIGS. 12(a), (b) and (c)). A second administration of FCIP within 6weeks (closed down triangles; 80 μg, FIG. 12(a)) demonstrated an attenuation of the response. Following administration of FC1P greater than one year after the initial dose (closed up triangles; 80 μg, FIGS. 12(a), (b) and (c)), a fall in FEV1 of similar magnitude to the initial injection was observed. Arrows indicate the use of rescue medication (β₂ agonists).

Schedule of sequences for sequence listing:

| | | |
|---|---|---|
| SEQ ID No 1: | LFLTGTPDEYVEQVAQY | (FC1P1) |
| SEQ ID No 2: | EQVAQYKALPVVLENA | (FC1P2) |
| SEQ ID No 3: | KALPVVLENARILKNCV | (FC1P3) |
| SEQ ID No 4: | Fel d I chain 1 in FIG. 9 | |
| SEQ ID No 5: | Fel d 2 chain 2 in FIG. 9 | |

Other SEQ ID Nos. for peptides are shown on FIG. 9.

### EXAMPLES

### Experimental Techniques

Primary Proliferation Assays

PBMCs are separated from whole blood by density gradient centrifugation according to standard methods. Cultures are established at 2×10⁵ cells per well in flat bottomed 96 well plates with 3 concentrations each individual peptide, or an optimum concentration of cat dander cat allergen extract, medium (negative control) or PPD (positive control). Cells are cultured for 8 days (cat dander) and 6 days (all others) and pulsed with 1 μCi tritiated thymidine. Cultures are harvested and counted after 8-16 hours.

T Cell Clones

PBMCs are cultured in 24 well plates with cat dander for 10-12 days, with the addition of approximately 10ng IL-2 on days 5 and 7, restimulated twice with irradiated autologous PBMCs and cat dander, and the line expanded with Phytohaemaglutinin (PHA) and IL-2. Clones are established by limiting dilution and will subsequently be frozen for use at a later stage to determine changes in cytokine secretion.

### Example 1

### Preparation of Allergen Peptides

The sequence of chain 1 of the cat allergen Fel d 1 is shown in FIG. 9 (SEQ. ID. No. 4); chain 2 is also shown in FIG. 9 (SEQ. ID. No. 5). Multiple overlapping peptides are designed around this sequence, as well as that of chain 2 of Fel d 1, as shown in FIG. 9.

### Example 2

### Observation of LAR in Patients on Peptide Administration

A single intradermal administration (80 μg of each peptide) of a mixture containing three short peptides (FIG. 9; (SEQ. ID Nos. 1, 2 or 3)) is given to 18 cat asthmatic individuals. 6 patients develop an isolated late asthmatic reaction as shown in FIG. 1 wherein a greater than 20% fall in Forced Expiratory Volume in 1 second (FEV1—a measure of lung function) is considered as a positive asthmatic effect. The results are shown in FIG. 1 where the three peptides comprising FC1P [solid circles; FC1P comprises FC1P1 (SEQ. ID. No. 1), FC1P2 (SEQ. ID. No. 2) and FC1P3 (SEQ. ID. No. 3)] or vehicle control (open circles) are injected intradermally at time zero on two separate days. FEV1 is measured at intervals as a readout of lung function over a 24 hr period. The use of rescue medication is indicated by arrows.

This result demonstrates that peptides capable of causing a LPR can be derived from a common allergen such as cat dander and tested for LAR production in cat asthmatic individuals.

Three patient volunteers who develop a late asthmatic reaction following administration of FC1P (closed circles), are challenged again with the same dose after a period of at least 2 weeks. No significant fall in FEV1 is observed following the second challenge (closed triangles). Open circles indicate the control day. Arrows indicate the use of bronchodilators. As shown in FIG. 2, none of the three develop a late asthmatic reaction to the second peptide administration indicating that the immune response to this peptide has been downregulated.

### Example 3

### Correlation Between Tissue Type and LAR

The 18 patients observed in Example 2 are MHC-typed using PCR, based upon the method of Olerup & Zetterquist (1992) Tissue Antigens 29:225-235. Four of the 6 reactors express HLA-DR13 (a closely related family of MHC molecules) compared to 1 out of 12 of the non-reactors. These results indicate that one of the three peptides injected is capable of binding to a DR13 family member and thus stimulating peptide-specific T cells from the reactors.

In order to demonstrate that specific T cells have been activated, L cells which have been transfected with the human genes encoding two DR13 family members are obtained from Georgetown University Medical School, USA. (DR13 is a split of DR6). Murine L cells expressing two DR13 variants, DRB1*1301 and 1302 are incubated overnight with each of the three FC1P peptides, or a control peptide, or medium alone. Cells are washed and incubated for one hour with a cytostatic agent to prevent proliferation in the subsequent assay. L cells are then incubated for 48 hours with T cells from a T cell line raised from PBMCs isolated from a reactor patient as described above and stimulated weekly with whole cat dander (and including the Fel d 1 protein). Proliferation of the T cells is measured by their incorporation of the radiolabelled compound ³H-thymidine. T cells demonstrate a statistically significant response to the DR13 L cells and peptide FC1P3 (SEQ. ID No 3) but not to the other peptides/control as shown in FIG. 3.

A further experiment is performed with human fibroblasts expressing the DR1 variant DRB1*0101. Cells are incubated overnight with each of the three FC1P peptides, or medium alone, washed, treated and incubated with T-cells as described above for the DR13 variants. T cells demonstrate a statistically significant response to the DR1 L cells and peptide FC1P3 (SEQ. ID No 3) but not to the other peptides/control as shown in FIG. 4

It is demonstrated halt FC1P3 is capable of binding to both DR1 and DR13 MHC molecules and activating T cells, thereby inducing the isolated late asthmatic reaction shown in FIG. 1. This result correlates extremely well with the tissue type data obtained from the patient population, wherein 4 out of six reactors are DR13 and two are DR1, compared with 1 out of 12 DR1 and 1 out of 12 DR13 non-reactors.

In a further series of experiments, patients reacting to FC1P are identified which express HLA-DR4 (DRB1*0405 and 0408). The same experiments are conducted as set forth above for HLA-DR13 patients, using DRB1*0404 and 0405 L-cells (0408 cells are not available). The results are shown in FIG. 5 and FIG. 6.

The results indicate that patients expressing DRB1* 0408 respond to FC1P2 presented by 0405 L cells but not 0404 L cells or to other peptides or controls. Likewise, patients expressing DRB1* 0405 respond to FC1P2 presented by 0405 L cells but not to other peptides or controls.

FIG. 7 shows the IL-5 secretion levels for DR13(a), DR1(b) and DR4(c) HLA types which correlate with T cell proliferation data as expected.

### Example 4

### FC1P3 Induces LAR and Desensitisation in Tissue-Typed Patients

Patients are selected on the basis of being allergic to cat dander, as in the previous examples. T-cell lines are prepared from each patient as described above, and maintained with weekly stimulation with cat dander extract. The patients are tissue-typed, and patients possessing DR1 or DR13 variants selected.

In order to predict the ability of peptide FC1P3 to desensitise the patients against cat dander, T-cell proliferation assays are performed using T-cells isolated from the patients as described and human fibroblasts or murine L-cells transfected with DR1 or DR13 alleles in the presence of FC1P3 according to Example 3. The T-cells are observed to proliferate, by the incorporation of ³H-thymidine, indicating that T-cells isolated from DR13 and DR1 possessing patients are responsive to stimulation with the FC1P3 peptide.

FC1P3 peptide is injected into patients which are DR1 and/or DR13 positive and in respect of whom a positive result has been obtained in the T-cell proliferation assay. These patients experience a LAR response, as measured by a 20% or greater fall in FEV1.

Patients who develop a late asthmatic reaction following administration of FC1P3 are challenged again with the same dose after a period of 2 weeks. As in Example 2. no significant fall or a reduced fall in FEV1 is observed following the second challenge, indicating that the immune response to this peptide has been downregulated.

### Example 5

### MHC Restriction Mapping of Fel d 1

In order to prepare a database of Fel d 1 derived peptides characterised according to MHC type restriction, an in vitro study of MHC class II restriction mapping is performed using a panel of L cells, T-cell lines to whole cat allergen and the overlapping peptides from chain 1 and chain 2, as described in Example 1. T cell lines with specificity for whole cat extract (which includes Fel d 1) are generated from the peripheral blood of subjects before peptide administration according to the procedures described above. Subjects are HLA-DR, DP and DQ typed, and, based on their expression, initially of DR alleles, transfected fibroblasts are selected to assay T-cell stimulation by each of the peptides.

Where the required HLA type clone is not available, MHC genes are cloned directly from the patient's cells by PCR amplification and cloning, as described above. Cloned genes are subsequently expressed in murine L-cells.

Cell lines (generous gifts from Prof. J. R. Lamb, University of Edinburgh, Prof. R. I. Lechler and Dr. G. Lombardi, ICSM, Hammersmith Hospital, Dr. C. Hurley and Dr. J. R. Richert, Georgetown University Medical Center. Washington, USA) expressing the appropriate restriction element are incubated with each individual Fel d 1 peptide as described in Example 3. Equivalent cell lines are generally available or may be readily made by transfecting appropriate genes expressing MHC Class II molecules. Following incubation in the cytostatic agent mitomycin C to prevent L cell division, cells are extensively washed and incubated with the T cell line. Proliferative responses are measured after 48 hours by addition of tritiated thymidine to all cultures for 8-16 hours. Peptides eliciting a proliferative response from the T cell line are thus restricted by the HLA allele expressed by the chosen L cell line.

Administration of peptides obtained from the database to patients possessing the HLA type in respect of which a proliferative response is seen in the above assay in the majority of cases results in a LAR, as expected, which is followed by desensitisation of the patient to cat dander on subsequent administration of the peptides.

In this way an MHC class II restriction map of the Fel d 1 molecule is constructed such that the appropriate peptides for immunotherapy may subsequently be selected on an individual patient basis, solely by virtue of that subject's HLA type.

### Example 6

### Identification of MHC-Restricted Peptides Capable of Inducing Late Phase Reactions in Individuals Possessing the Appropriate MHC Molecule

(1) Overlapping peptides of 15 amino acid residues (range approx. 7-20) which are offset by one residue are chemically synthesised for example using FastMoc chemistry. An example of a hypothetical protein and the overlapping peptides (in this example 15mers) which may be derived from it is given in FIG. 8.

(2) Each individual peptide is incubated with murine or human cells such as fibroblasts for example, which have been transfected with, or already express, the genes encoding a particular MHC molecule such as, for example DRA and DRB1*0101. The concentration of peptide used for the incubation stage may vary from approximately 0.01 mg/ml to 1 mg/ml or more. An example is 200 μg/ml. The incubation period may vary from approximately a few minutes to several hours. An example is 16 hours.

(3) Following incubation with peptide, the cells are washed several times (for example 3 times) in tissue culture medium (for example RPMI-1640 medium supplemented with 5% normal human AB serum, 2 mM L-glutamine, 100 microgram/ml streptomycin and 100U/ml penicillin).

(4) Cells are then incubated with mitomycin C (at approximately 50 μg/ml) or another suitable cytostatic agent to prevent cell division. Cells are washed several times (for example 5 times) in culture medium and dispensed into 96 well tissue culture plates at a concentration of approximately 3×10⁴ cells per well for example.

(5) To these cells are added approximately 1×10⁴ cells of a T lymphocyte cell line which has been cultured in the presence of, and is reactive with, the protein from which the peptides in step (1) were derived. The MHC molecules expressed by the individual from which the T lymphocyte line was raised would usually include the MHC molecule expressed on the cells in step (2). Alternatively, the MHC molecules expressed by the individual from whom the T lymphocyte line was raised may differ from those expressed on the cells in step (2). Additionally, T lymphocytes from the same cell line are cultured on their own and also with the MHC-expressing cells described in stage (2) which have either not been incubated with a peptide, or have been incubated with an irrelevant peptide such as a peptide from another protein.

6) The cell mixture is cultured for approximately 2-3 days prior to the addition to each well of approximately 37MBq (1 μCi) of tritiated thymidine or similar for several hours (for example 6-16 hours).

7) Cultures are then harvested onto glass fibre filters and cellular proliferation (of the T lymphocytes), as correlated with uptake of tritiated thymidine into the DNA of the cells, is measured by liquid scintillation spectroscopy or a similar technique.

Peptides capable of binding to the relevant MHC molecules and inducing T cell activation are identified by the incorporation of the tritiated thymidine into the newly synthesised DNA of the activated T cells. When the DNA is analysed by liquid scintillation spectroscopy (or other suitable techniques) the radioactive label (tritium) generated counts per minute which correlate with the degree of T cell proliferation and thus activation.

Thus, MOPs derived from a polypeptide allergen are useful principally in the selection procedure for identifying the one or more useful peptides (which show MHC Class II restriction and which are able to give rise to a LPR in an individual who possesses the appropriate MHC Class II molecules) which may be used either individually or in combination as an immunotherapeutic agent.

The following is a list of known allergen sequences and database accession numbers (NCBI Entrez accession numbers). NCBI is the National Center for Biotechnology information and is a division of the US National Institutes of Health. The NCBI web site, from which access to the database may be sought, www.ncbi.nlm.nih.gov/. The allergens may be used as described above in order to identify MHC-restricted peptides capable of inducing LPR in individuals who possess a particular MHC molecule.

Allergen sequences and database accession numbers (NCBI Entrez accession numbers):

House dust mite
Dermatophagoides pteronyssinus
Der p 1
MKIVLAIASLLALSAVYARPSSIKTFEEYKKAFNKSYATFEDEEAAR
KNFLESVKYVQSNGGAINHLSDLSLDEFKNRFLMSAEAFEHLKTQFD
LNAETNACSINGNAPAEIDLRQMRTVTPIRMQGGCGSCWAFSGVAAT
ESAYLAYRNQSLDLAEQELVDCASQHGCHGDTIPRGIEYIQHNGVVQ
ESYYRYVAREQSCRRPNAQRFGISNYCQIYPPNVNKIREALAQTHSA
IAVIIGIKDLDAFRHYDGRTIIQRDNGYQPNYHAVNIVGYSNAQGVD
YYWIVRNSWDTNWGDNGYGYFAANIDLMMIEEYPYVVIL
Der p 2
MMYKILCLSLLVAAVARDQVDVKDCANHEIKKVLVPGCHGSEPCIIH
RGKPFQLEAVFEANQNTKTAKIEIKASIDGLEVDVPGIDPNACHYMK
CPLVKGQQYDIKYTWNVPKIAPKSENVVVTVKVMGDDGVLACAIATH
AKIRD
Der p 3
MIIYNILIVLLLAINTLANPILPASPNATIVGGEKALAGECPYQISL
QSSSHFCGGTILDEYWILTAAHCVAGQTASKLSIRYNSLKHSLGGEK
ISVAKIFAHEKYDSYQIDNDIALIKLKSPMKLNQKNAKAVGLPAKGS
DVKVGDQVRVSGWGYLEEGSYSLPSELRRVDIAVVSRKECNELYSKA
NAEVTDNMICGGDVANGGKDSCQGDSGGPVVDVKNNQVVGIVSWGYG
CARKGYPGVYTRVGNFIDWIESKRSQ
Der p 4
KYXNPHFIGXRSVITXLME
Der p 5
MKFIIAFFVATLAVMTVSGEDKKHDYQNEFDFLLMERIHEQIKKGEL
ALFYLQEQINHFEEKPTKEMKDKIVAEMDTIIAMIDGVRGVLDRLMQ
RKDLDIFEQYNLEMAKKSGDILERDLKKEEARVKKIEV
Der p 6
AIGXQPAAEAEAPFQISLMK
Der p 7
MMKLLLIAAAAFVAVSADPIHYDKITEEINKAVDEAVAAIEKSETFD
PMKVPDHSDKFERHIGIIDLKGELDMRNIQVRGLKQMKRVGDANVKS
EDGVVKAHLLVGVHDDVVSMEYDLAYKLGDLHPNTHVISDIQDFVVE
LSLEVSEEGNMTLTSFEVRQFANVVNHIGGLSILDPIFAVLSDVLTA
IFQDTVRAEMTKVLAPAFKKELERNNQ
Der p 9
IVGGSNASPGDAVYQIAL
Dermatophagoides farinae
Der f 1
MKFVLAIASLLVLTVYARPASIKTFEFKKAFNKNYATVEEEEVARKN
FLESLKYVEANKGAINHLSDLSLDEFKNRYLMSAEAFEQLKTQFDLN
AETSACRINSVNVPSELDLRSLRTVTPIRMQGGCGSCWAFSGVAATE
SAYLAYRNTSLDLSEQELVDCASQHGCHGDTIPRGIEYIQQNGVVEE
RSYPYVAREQRCRRPNSQHYGISNYCQIYPPDVKQIREALTQTHTAI
AVIIGIKDLRAFQHYDGRTIIQHDNGYQPNYHAVNIVGYGSTQGDDY
WIVRNSWDTTWGDSGYGYFQAGNNLMMIEQYPYVVIM
Der f 2
MISKILCLSLLVAAVVADQVDVKDCANNEIKKVMVDGCHGSDPCIIH
RGKPFTLEALFDANQNTKTAKIEIKASLDGLEIDVPGIDTNACHFMK
CPLVKGQQYDIKYTWNVPKIAPKSENVVVTVKLIGDNGVLACAIATH
GKIRD
Der f 3
MMILTIVVLLAANILATPILPSSPNATIVGGVKAQAGDCPYQISLQS
SSHFCGGSILDEYWILTAAHCVNGQSAKKLSIRYNTLKHASGGEKIQ
VAEIYQHENYDSMTIDNDVALIKLKTPMTLDQTNAKPVPLPAQGSDV
KVGDKIRVSGWGYLQEGSYSLPSELQRVDIDVVSREQCDQLYSKAGA
DVSENMICGGDVANGGVDSCQGDSGGPVVDVATKQIVGIVSWGYGCA
RKGYPGVYTRVGNPVDWIESKRSQ
Der f 4
AVGGQDADLAEAPFQISLLK
Der f 7
MMKFLLIAAVAFVAVSADPIHYDKITEEINKAIDDAIAAIEQSETID
PMKVPDHADKFERHVGIVDFKGELAMRNIEARGLKQMKRQGDANVKG
EEGIVKAHLLIGVHDDIVSMEYDLAYKLGDLHPTTHVISDIQDFVVA
LSLEISDEGNITMTSFEVRQFANVVNHIGGLSILDPIFGVLSDVLTA
IFQDTVRKEMTKVLAPAFKRELEKN

Additional mite allergen sequences (NCBI entrez accession):

1170095; 1359436; 2440053; 666007; 487661; 1545803; 84702; 84699; 625532; 404370; 1091577; 1460058; 7413; 9072; 387592.

Cat
Felis sequences
1082946 Fel dI chain 2 precursor-cat
MRGALLVLALLVTQALGVKMAETCPIFYDVFFAVANGNELLLDLS
LTKVNATEPERTAMKKIQDCYVENGLISRVLDGLVMTTISSSKDC
MGEAVQNTVEDLKLNTLGR
1082945 Fel dI chain 1 short form-cat
MLDAALPPCPTVAATADCEICPAVKRDVDLFLTGTPDEYVEQVAQ
YKALPVVLENARILKNCVDAKMTEEDKENALSLLDKIYTSPLC
1082944 Fel dI chain 1 long form precursor-cat
MKGARVLVLLWAALLLIWGGNCEICPAVKRDVDLFLTGTPDEYVE
QVAQYKALPVVLENARILKNCVDAKMTEEDKENALSLLDMYTSPL
C

Additional Felis sequences (NCBI entrez accession):

539716; 539715; 423193; 423192; 423191; 423190; 1364213; 1364212; 395407; 163827; 1638923 163825; 1169665; 232086; 1169666.

Latex
Hevea sequences:
Hev b 1
MAEDEDNQQGQGEGLKYLGFVQDAATYAVTTFSNVYLFAKDKSG
PLQPGVDIIEGPVKNVAVPLYNRFSYIPNGALKFVDSTVVASVT
IIDRSLPPIVKDASIQVVSAIRAAPEAARSLASSLPGQTKILAK
VFYGEN
Hev b 3
MAEEVEEERLKYLDFVRAAGVYAVDSFSTLYLYAKDISGPLKPG
VDTIENVVKTVVTPVYYIPLEAVKFVDKTVDVSVTSLDGVVPPV
IKQVSAQTYSVAQDAPRIVLDVASSVFNTGVQEGAKALYANLEP
KAEQYAVITWRALNKLPLVPQVANVVVPTAVYFSEKYNDVVRGT
TEQGYRVSSYLPLLPTEKITKVFGDEAS

Additional Hevea sequences (NCBI entrez accession):

3319923; 3319921; 3087805; 1493836; 1480457; 1223884; 3452147; 3451147; 1916805; 232267; 123335; 2501578; 3319662; 3288200; 1942537; 2392631; 2392630; 1421554; 1311006; 494093; 3183706; 3172534; 283243; 1170248; 1708278; 1706547; 464775; 266892; 231586; 123337, 116359; 123062; 2213877; 542013; 2144920; 1070656; 2129914; 2129913; 2129912; 100135; 82026; 1076559; 82028; 82027; 282933; 280399; 100138; 1086972; 108697; 1086976; 1086978; 1086978; 1086976; 1086974; 1086972; 913758; 913757; 913756; 234388; 1092500; 228691; 1177405; 18839; 18837; 18835; 18833; 18831; 1209317; 1184668; 168217; 168215; 168213; 168211; 168209; 348137.

Rye grass
Lolium sequences:
126385 Lol p 1
MASSSSVLLVVALFAVFLGSAHGIAKVPPGPNITAEYGDKWLDAKS
TWYGKPTGAGPKDNGGACGYKNVDKAPFNGMTGCGNTPIFKDGRGC
GSCFEIKCTKPESCSGEAVTVTITDDNEEPIAPYHFDLSGHAFGSM
AKKGEEQNVRSAGELELQFRRVKCKYPDDTKPTFHVEKASNPNYLA
ILVKYVDGDGDVVAVDIKEKGKDKWIELKESWGAVWRIDTPDKLTG
PFTVRYTTEGGTKSEFEDVIPEGWKADTSYSAK
126386 Lol p 2a
AAPVEFTVEKGSDEKNLALSIKYNKEGDSMAEVELKEHGSNEWLAL
KKNGDGVWEIKSDKPLKGPFNFRFVSEKGMRNVFDDVVPADFKVGT
TYKPE
126387 Lol p 3
TKVDLTVEKGSDAKTLVLNIKYTRPGDTLAEVELRQHGSEEWEPMT
KGNLWEVKSAKPLTGPMNFRFLSKGGMKNVFDEVIPTAFTVGKTYT
PEYN
2498581 Lol p 5a
MAVQKYTVALFLRRGPRGGPGRSYAADAGYTPAAAATPATPAATPA
GGWREGDDRRAEAAGGRQRLASRQPWPPLPTPLRRTSSRSSRPPSP
SPPRASSPTSAAKAPGLIPKLDTAYDVAYKAAEAHPRGQVRRLRHC
PHRSLRVIAGALEVHAVKPATEEVLAAKIPTGELQIVDKIDAAFKI
AATAANAAPTNDKFTVFESAFNKALNECTGGAMRPTSSSPPSRPRS
SRPTPPPSPAAPEVKYAVFEAALTKAITAMTQAQKAGKPAAAAATA
AATVATAAATAAAVLPPPLLVVQSLISLLIYY
2498582 Lol p 5b
MAVQKHTVALFLAVALVAGPAASYAADAGYAPATPATPAAPATAAT
PATPATPATPAAVPSGKATTEEQKLIEKINAGFKAAVAAAAVVPPA
DKYKTFVETFGTATNKAFVEGLASGYADQSKNQLTSKLDAALKLAY
EAAQGATPEAKYDAYVATLTEALRVIAGTLEVHAVKPAAEEVKVGA
IPAAEVQLIDKVDAAYRTAATAANAAPANDKFTVFENTFNNAIKVS
LGAAYDSYKFIPTLVAAVKQAYAAKQATAPEVKYTVSETALKKAVT
AMSEAEKEATPAAAATATPTPAAATATATPAAAYATATPAAATATA
TPAAATATPAAAGGYKV
455288 Lol p isoform 9
MAVQKHTVALFLAVALVAGPAASYAADAGYAPATPATPAAPATAAT
PATPATPATPAAVPSGKATTEEQKLIEKINAGFKAAVAAAAVVPPA
DKYKTFVETPGTATNKAFVEGLASGYADQSKNQLTSKLDAALKLAY
EAAQGATPEAKYDAYVATLTEALRVIAGTLEVHAVKPAAEEVKVGA
IPAAEVQLIDKVDAAYRTAATAANAAPANDKFTVFENTFNNAIKVS
LGAAYDSYKFIPTLVAAVKQAYAAKQATAPEVKYTVSETALKKAVT
AMSEAEKEATPAAAATATPTPAAATATATPAAAYATATPAAATATA
TPAAATATPAAAGGYKV
1582249 Lol p 11
DKGPGFVVTGRVYCDPCRAGFETNVSHNVEGATVAVDCRPFDGGES
KLKAEATTDKDGWYKIEIDQDHQEEICEVVLAKSPDKSCSEIEEFR
DRARVPLTSNXGIKQQGIRYANPIAFFRKEPLKECGGILQAY

Additional Lolium sequences (NCBI entrez accession):

135480; 417103; 687261; 687259; 1771355; 2388662; 631955; 542131; 542130; 542129; 100636; 626029; 542132; 320616; 320615; 320614; 100638; 100634; 82450; 626028; 100639; 283345; 542133; 1771353: 1763163; 1040877; 1040875; 250525; 551047; 515377; 510911; 939932; 439950; 2718; 168316; 168314; 485371; 2388664; 2832717; 2828273; 548867.

Olive tree
Olive sequences
416610 Ole e 1
EDIPQPPVSQFHIQGQVYCDTCRAGFITELSEFIPGASLRLQCKDKEN
GDVTFTEVGYTRAEGLYSMLVE
RDHKNEFCEITLISSGRKDCNEIPTEGWAKPSLKFKLNTVNGTTRTVN
PLGFFKKEALPKCAQVYNKLGM
YPPNM
Parietaria
Parietaria sequences:
2497750 Par j P2
MRTVSMAALVVIAAALAWTSSAEPAPAPAPGEEACGKVVQDIMPCLHF
VKGEEKEPSKECCSGTKKLSEEVKTTEQKREACKCIVRATKGISGIKN
ELVAEVPKKCDIKTTLPPITADFDCSKIQSTIFRGYY
1352506 Par j P5
MVRALMPCLPFVQGKEKEPSKGCCSGAKRLDGETKTGPQRVHACECIQ
TAMKTYSDIDGKLVSEVPKHCGIVDSKLPPIDVNMDCKTVGVVPRQPQ
LPVSLRHGPVTGPSDPAHKARLERPQIRVPPPAPEKA
1532056 Par j P8
MRTVSMAALVVIAAALAWTSSAELASAPAPGEGPCGKVVHHIMPCLKF
VKGEEKEPSKSCCSGTKKLSEEVKTTEQKREACKCIVAATKGISGIKN
ELVAEVPKKCGITTTLPPITADFDCSKIESTIFRGYY
1532058 Par j P9
MRTVSAPSAVALVVIVAAGLAWTSLASVAPPAPAPGSEETCGTVVRAL
MPCLPFVQGKEKEPSKGCCSGAKRLDGETKTGLQRVHACECIQTAMKT
YSDIDGKLVSEVPKHCGIVDSKLPPIDVNMDCKTLGVVPRQPQLPVSL
RHGPVTGPSDPAHKARLERPQIRVPPPAPEKA
2497749 Par j P9
MRTVSARSSVALVVIVAAVLVWTSSASVAPAPAPGSEETCGTVVGALM
PCLPFVQGKEKEPSKGCCSGAKRLDGETKTGPQRVHACECIQTAMKTY
SDIDGKLVSEVPKHCGIVDSKLPPIDVNMDCKTLGVLHYKGN
1086003 Par j 1
MVRALMPCLPFVQGKEKEPSKGCCSGAKRLDGETKTGPQRVHACECIQ
TAMKTYSDIDGKLVSEVPKHCGIVDSKLPPIDVNMDCKTVGVVPRQPQ
LPVSLRHGPVTGPSRSRPPTKHGWRDPRLEFRPPHRKKPNPAFSTLG

Additional Parietaria sequences (NCBI entrez accession):

543659; 1836011; 1836010; 1311513; 1311512; 1311511; 1311510; 1311509; 240971.

Timothy grass
Phleum sequences:
Phl p 1
MASSSSVLLVVVLFAVFLGSAYGIPKVPPGPNITATYGDKWLDAKS
TWYGKPTGAGPKDNGGACGYKDVDKPPFSGMTGCGNTPIFKSGRGC
GSCFEIKCTKPEACSGEPVVVHITDDNEEPIAPYHFDLSGHAFGAM
AKKGDEQKLRSAGELELQFRRVKCKYPEGTKVTFNVEKGSNPNYLA
LLVKYVNGDGDVVAVDIKEKGKDKWIELKESWGAIWRIDTPDKLTG
PFTVRYTTEGGTKTEAEDVIPEGWKADTSYESK
Phl p 1
MASSSSVLLVVALFAVFLGSAHGIPKVPPGPNITATYGDKWLDAKS
TWYGKPTAAGPKDNGGACGYKDVDKPPFSGMTGCGNTPIFKSGRGC
GSCFEIKCTKPEACSGEPVVVHITDDNEEPIAAYHFDLSGIAFGSM
AKKGDEQKLRSAGEVEIQFRRVKCKYPEGTKVTFHVEKGSNPNYLA
LLVKFSGDGDVVAVDIKEKGKDKWIALKESWGAIWRIDTPEVLKGP
FTVRYTTEGGTKARAKDVIPEGWKADTAYESK
Phl p 2
MSMASSSSSSLLAMAVLAALFAGAWCVPKVTFTVEKGSNEKHLAVL
VKYEGDTMAEVELREHGSDEWVAMTKGEGGVWTFDSEEPLQGPFNF
RFLTEKGMKNVFDDVVPEKYTIGATYAPEE
Phl p 5
ADLGYGGPATPAAPAEAAPAGKATTEEQKLIEKINDGFAALAAAAG
VPPADKYKTFVATFGAASNKAFAEGLSAEPKGAAESSSKAALTSKL
DAAYKLAYKTAEGATPEAKYDAYVATLSEALRIIAGTLEVHAVKPA
AEEVKVIPAGELQVIEKVDSAFKVAATAANAAPANDKFTVFEAAFN
NAIKASTGGAYESYKFIPALEAAVKQAYAATVATAPEVKYTVFETA
LKKAFTAMSEAQKAAKPATEATATATAAVGAATGAATAATGGYKV
Phl p 5
ADLGYGGPATPAAPAEAAPAGKATTEEQKLIEKINDGFKAALAAAA
GVPPADKYKTFVATFGAASNKAFAEGLSAEPKGAAESSSKAALTSK
LDAAYKLAYKTAEGATPEAKYDAYVATLSEALRIIAGTLEVHAVKP
AAEEVKVIPAGELQVIEKVDSAFKVAATAANAAPANDKFTVFEAAF
NNAIKASTGGAYESYKFIPALEAAVKQAYAATVATAPEVKYTVFET
ALKKAITAMSEAQKAAKPATEATATATAAVGAATGAATAATGGYKV
Phl p 5b
AAAAVPRRGPRGGPGRSYTADAGYAPATPAAAGAAAGKATTEEQKL
IEDINVGFKAAVAAAASVPAADKFKTFEAAFTSSSKAAAAKAPGLV
PKLDAAYSVAYKAAVGATPEAKFDSFVASLTEALRVIAGALEVHAV
KPVTEEPGMAKIPAGELQIIDMDAAFKVAATAAATAPADDKFTVFE
AAFNKAIKESTGGAYDTYKCIPSLEAAVKQAYAATVAAAPQVKYAV
FEAALTKAITAMSEVQKVSQPATGAATVAAGAATTAAGAASGAATV
AAGGYKV
Phl p 5a
ADLGYGPATPAAPAAGYTPATPAAPAGADAAGKATTEEQKLIEKIN
AGFKAALAGAGVQPADKYRTFVATFGPASNKAFAEGLSGEPKGAAE
SSSKAALTSKLDAAYKLAYKTAEGATPEAKYDAYVATLSEALRIIA
GTLEVHAVKPAAEEVKVIPAGELQVIEKVDAAFKVAATAANAAPAN
DKFTVFEAAFNDEIKASTGGAYESYKFIPALEAAVKQAYAATVATA
PEVKYTVFETALKKAITAMSEAQKAAKPAAAATATATAAVGAATGA
ATAATGGYKV
Phl p 5
MAVQKYTVALFLAVALVAGPAASYAADAGYAPATPAAAGAEAGKAT
TEEQKLIEDINVGFKAAVAAAASVPAADKFKTFEAAFTSSSKAATA
KAPGLVPKLDAAYSVSYKAAVGATPEAKFDSFVASLTEALRVIAGA
LEVHAVKPVTEEPGMAKIPAGELQIIDKIDAAFKVAATAAATAPAD
TVFEAAFNKAIKESTGGAYDTYKCIPSLEAAVKQAYALATVAAAPQ
VKYAVFEAALTKAITAMSEVQKVSQPATGAATVAAGAATTAAGAAS
GAATVAAGGYKV
Phl p 5
MAVQKYTVALFLAVALVAGPAASYAADAGYAPATPAAAGAEAGKAT
TEEQKLIEDINVGFAVAAAASVPAADKFKTFEAAFTSSSKAATAKA
PGLVPKLDAAYSVAYKAAVGATPEAKFDSFVASLTEALRVIAGALE
VHAVKPVTEDPAWPKIPAGELQIIDKIDAAFKVAATAAATAPADDK
FTVFEAAFNKAIKESTGGAYDTYKCIPSLEAAVKQAYAATVAAAPQ
VKYAVFEAALTKAITAMSEVQKVSQPATGAATVAAGAATTATGAAS
GAATVAAGGYKV
Phl p 5
ADAGYAPATPAAAGAEAGKATTEEQKLIEDINVGFKAAVAAAASVP
AADKFKTFEAAFTSSSKAATAKAPGLVPKLDAAYSVAYKAAVGATP
EAKFDSFVASLTEALRVIAGALEVHAVKPVTEEPGMAKIPAGELQI
IDKIDAAFKVAATAAATAPADDKFTVFEAAFNKAIKESTGGAYDTY
KCIPSLEAAVKQAYAATVAAAPQVKYAVFEAALTKAITAMSEVQKV
SQPATGAATVAAGAATTAAGAASGAATVAAGGYKV
Phl p 5
SVKRSNGSAEVHRGAVPRRGPRGGPGRSYAADAGYAPATPAAAGAE
AGKATTEEQKLIEDINVGFKAAVAAAASVPAADKFKTFEAAFTSSS
KAATAKAPGLVPKLDAAYSVAYKAAVGATPEAKFDSFVASLTEALR
VIAGALEVHAVKPVTEEPGMAKIPAGELQIIDKIDAAFKVAATAAA
TAPADDKFTVFEAAFNKAIKESTGGAYDTYKCIPSLEAAVKQAYAA
TVAAAPQVKYAVFEAALTKAITAMSEVQKVSQPATGAATVAAGAAT
TAAGAASGAATVAAGGYKV
Phl p 5
MAVHQYTVALFLAVALVAGPAGSYAADLGYGPATPAAPAAGYTPAT
PAAPAGAEPAGKATEEQKLIEKINAGFKAALAAAAGVPPADKYRTF
VATFGAASNKAFAEGLSGEPKGAAESSSKAALTSKLDAAYKLAYKT
AEGATPEAKYDAYVATVSEALRIIAGTLEVHAVKPAAEEVKVIPAG
ELQVIEKVDAAFKVAATAANAAPANDKFTVFEAAFNDAIKASTGGA
YESYKFIPALEAAVKQAYAATVATAPEVKYTVFETALKKAITAMSE
AQKAAKPAAAATATATAAVGAATGAATAATGGYKV
Phl p 5
ADLGYGGPATPAAPAEAAPAGKATTEEQKLIEKINDGFKAALAAAA
GVPPADKYKTFVATFGAASNKAFAEGLSAEPKGAAESSSKAALTSK
LDAAYKLAYKTAEGATPEAKYDAYVATLSEALRIIAGTLEVHAVKP
AAEEVKVIPAGELQVIEKVDSAFKVAATAANAAPANDKFTVFEAAF
NNAIKASTGGAYESYKFIPALEAAVKQAYAATVATAPEVKYTVFET
ALKKAFTAMSEAQKAAKPATEATATATAAVGAATGAATAATGGYKV
Phl p 5b
AAAAVPRRGPRGGPGRSYTADAGYAPATPAAAGAAAGKATTEEQKL
IEDINVGFKAAVAAAASVPAADKFKTFEAAFTSSSKAAAAKAPGLV
PKLDAAYSVAYKAAVGATPEAKFDSFVASLTEALRVIAGALEVHAV
KPVTEEPGMAKIPAGELQIIDKIDAAFKVAATAAATAPADDKFTVF
EAAFNKAIKESTGGAYDTYKCIPSLEAAVKQAYAATVAAAPQVKYA
VFEAALTKAITAMSEVQKVSQPATGAATVAAGAATTAAGAASGAAT
VAAGGYKV
Phl p 5a
ADLGYGPATPAAPAAGYTPATPAAPAGADAAGKATTEEQKLIEKIN
AGFKAALAGAGVQPADKYRTFVATFGPASNKAFAEGLSGEPKGAAE
SSSKAALTSKLDAAYKLAYKTAEGATPEAKYDAYVATLSEALRIIA
GTLEVHAVKPAAEEVKVIPAGELQVIEKVDAAFKVAATAANAAPAN
DKFTVFEAAFNDEIKASTGGAYESYKFIPALEAAVKQAYAATVATA
PEVKYTVFETALKKAITAMSEAQKAAKPAAAATATATAAVGAATGA
ATAATGGYKV
Phl p 5
AVPRRGPRGGPGRSYAADAGYAPATPAAAGAEAGKATTEEQKLIED
INVGFKAAVAAAASVPAGDKFKTFEAAFTSSSKAATAKAPGLVPKL
DAAYSVAYKAAVGATPEAKFDSFVASLTEALRVIAGALEVHAVKPV
TEEPGMAKIPAGELQIIDKIDAAFKVAATAAATAPADDKFTVFEAA
FNKAIKESTGGAYDTYKCIPSLEAAVKQAYAATVAAAPQVKYAVFE
AALTKAITAMSEVQKVSQPATGAATVAAGAATTATGAASGAATVAA
GGYKV
Phl p 5b
MAVPRRGPRGGPGRSYTADAGYAPATPAAAGAAAGKATTEEQKLIE
DINVGFKAAVAARQRPAADKFKTFEAASPRHPRPLRQGAGLVPKLD
AAYSVAYKAAVGATPEAKFDSFVASLTEALRVIAGALEVHAVKPVT
EEPGMAKIPAGELQIIDKIDAAFKVAATAAATAPADDKFTVFEAAF
NKAIKESTGGAYDTYKCIPSLEAAVKQAYAATVAAAAEVKYAVFEA
ALTKAITAMSEVQKVSQPATGAATVAAGAATTAAGAASGAATVAAG
GYKV
Phl p 5
MAVHQYTVALFLAVALVAGPAASYAADLGYGPATPAAPAAGYTPAT
PAAPAEAAPAGKATTEEQKLIEKINAGFKAALAAAAGVQPADKYRT
FVATFGAASNKAFAEGLSGEPKGAAESSSKAALTSKLDAAYKLAYK
TAEGATPEAKYDAYVATLSEALRIIAGTLEVHAVKPAAEEVKVIPA
GELQVIEKVDAAFKVAATAANAAPANDKFTVFEAAFNDAIKASTGG
AYESYKFIPALEAAVKQAYAATVATAPEVKYTVFETALKKAITAMS
EAQKAAKPAAAATATATAAVGAATGAATAATGGYKV
Phl p 5
EAPAGKATTEEQKLIEKINAGFKAALARRLQPADKYRTFVATFGPA
SNKAFAEGLSGEPKGAAESSSKAALTSKLDAAYKLAYKTAEGATPE
AKYDAYVATLSEALRIIAGTLEVHAVKPAAEEVKVIPAAELQVIEK
VDAAFKVAATAANAAPANDKFTVFEAAFNDEIKASTGGAYESYKFI
PALEAAVKQAYAATVATAPEVKYTVFETALKKAITAMSEAQKAAKP
PPLPPPPQPPPLAATGAATAATGGYKV
Phl p 5
MAVHQYTVALFLAVALVAGPAASYAADLGYGPATPAAPAAGYTPAT
PAAPAEAAPAGKATTEEQKLIEKINAGFKAALAAAAGVQPADKYRT
FVATFGAASNKAFAEGLSGEPKGAAESSSKAALTSKLDAAYKLAYK
TAEGATPEAKYDAYVATLSEALRIIAGTLEVHAVKPAAEEVKVIPA
GELQVIEKVDAAFKVAATAANAAPANDKFTVFEAAFNDAIKASTGG
AYESYKFIPALEAAVKQAYAATVATAPEVKYTVFETALKKAITAMS
EAQKAAKPAAAATATATAAVGAATGAATAATGGYKV
Phl p 5b
MAVPRRGPRGGPGRSYTADAGYAPATPAAAGAAAGKATTEEQKLIE
DINVGFKAAVAARQRPAADKFKTFEAASPRHPRPLRQGAGLVPKLD
AAYSVAYKAAVGATPEAKFDSFVASLTEALRVIAGALEVHAVKPVT
EEPGMAKIPAGELQIIDKIDAAFKVAATAAATAPADDKFTVFEAAF
NKAIKESTGGAYDTYKCIPSLEAAVKQAYAATVAAAAEVKYAVFEA
ALTKAITAMSEVQKVSQPATGAATVAAGAATTAAGAASGAATVAAG
GYKV
Phl p 5a
ADLGYGPATPAAPAAGYTPATPAAPAGADAAGKATTEEQKLIEKIN
AGFKAALAGAGVQPADKYRTFVATFGPASNKAFAEGLSGEPKGAAE
SSSKAALTSKLDAAYKLAYKTAEGATPEAKYDAYVATLSEALRIIA
GTLEVHAVKPAAEEVKVIPAGELQVIEKVDAAFKVAATAANAAPAN
DKFTVFEAAFNDEIKASTGGAYESYKFIPALEAAVKQAYAATVATA
PEVKYTVFETALKKAITAMSEAQKAAKPPPLPPPPQPPPLAATGAA
TAATGGYKV
Phl p 5
MAVHQYTVALFLAVALVAGPAASYAADLGYGPATPAAPAAGYTPAT
PAAPAEAAPAGKATTEEQKLIEKINAGFKAALAAAAGVQPADKYRT
FVATFGAASNKAFAEGLSGEPKGAAESSSKAALTSKLDAAYKLAYK
TAEGATPEAKYDAYVATLSEALRIIAGTLEVHAVKPAAEEVKVIPA
GELQVIEKVDAAFKVAATAANAAPANDKFTVFEAAFNDAIKASTGG
AYESYKFIPALEAAVKQAYAATVATAPEVKYTVFETALKKAITAMS
EAQKAAKPAAAATATATAAVGAATGAATAATGGYKV
Phl p 6
MAAHKFMVAMFLAVAVVLGLATSPTAEGGKATTEEQKLIEDVNASF
RAAMATTANVPPADKYKTFEAAFTVSSKRNLADAVSKAPQLVPKLD
EVYNAAYNAADHAAPEDKYEAFVLHFSEALRIIAGTPEVHAVKPGA
Phl p 6
SKAPQLVPKLDEVYNAAYNAADHAAPEDKYEAFVLHFSEALHIIAG
TPEVHAVKPGA
Phl p 6
ADKYKTFEAAFTVSSKRNLADAVSKAPQLVPKLDEVYNAAYNAADH
AAPEDKYEAFVLHFSEALHIIAGTPEVHAVKPGA
Phl p 6
TEEQKLIEDVNASFRAAMATTANVPPADKYKTLEAAFTVSSKRNLA
DAVSKAPQLVPKLDEVYNAAYNAADHAAPEDKYEAFVLHFSEALRI
IAGTPEVHAVKPGA
Phl p 6
MAAHKFMVAMFLAVAVVLGLATSPTAEGGKATTEEQKLIEDINASF
RAAMATTANVPPADKYKTFEAAFTVSSYKRNLADAVSKAPQLVPKL
DEVYNAAYNAADHAAPEDKYEAFVLHFSEALHIIAGTPEVHAVKPG
A
Phl p 6
MVAMFLAVAVVLGLATSPTAEGGKATTEEQKLIEDVNASFRAAMAT
TANVPPADKYKTFEAAFTVSSKRNLADAVSKAPQLVPKLDEVYNAA
YNAADHAAPEDKYEAFVLHFSEALRIIAGTPEVHAVKPGA
Phl p 7
MADDMERIFKRFDTNGDGKISLSELTDALRTLGSTSADEVQRMMAE
IDTDGDGFIDFNEFISFCNANPGLMKDVAKVF
Phl p 11
MSWQTYVDEHLMCEIEGHHLASAAILGHDGTVWAQSADFPQFKPEE
ITGIMKDFDEPGHLAPTGMFVAGAKYMVIQGEPGRVIRGKKGAGGI
TIKKTGQALVVGIYDEPMTPGQCNMVVERLGDYLVEQGM

Additional Phleum sequences (NCBI entrez accession):

458878; 548863; 2529314; 2529308; 2415702; 2415700; 2415698; 542168; 542167; 626037; 542169; 541814; 542171; 253337; 253336; 453976; 439960.

Wasp (and related)
Vespula sequences:
465054 ALLERGEN VES V 5
MEISGLVYLIIIVTIIDLPYGKANNYCKIKCLKGGVHTACKYGSLKPN
CGNKVVVSYGLTKQEKQDILKEHNDFRQKIARGLETRGNPGPQPPAKN
MKNLVWNDELAYVAQVWANQCQYGHDTCRDVAKYQVGQNVALTGSTAA
KYDDPVKLVKMWEDEVKDYNPKKKFSGNDFLKTGHYTQMVWANTKEVG
CGSIKYIQEKWHKHYLVCNYGPSGNFMNEELYQTK
1709545 ALLERGEN VES M 1
GPKCPFNSDTVSIIIETRENRNRDLYTLQTLQNHPEFKKKTITRPVVF
ITHGFTSSASEKNFINLAKALVDKDNYMVISIDWQTAACTNEYPGLKY
AYYPTAASNTRLVGQYIATITQKLVKDYKISMANIRLIGHSLGAHVSG
FAGKRVQELKLGKYSEIIGLDPARPSFDSNHCSERLCETDAEYVQIIH
TSNYLGTEKILGTVDFYMNNGKNNPGCGRFFSEVCSHTRAVIYMAECI
KHECCLIGIPRSKSSQPISRCTKQECVCVGLNAKKYPSRGSFYVPVES
TAPFCNNKGKII
1352699 ALLERGEN VES V 1
MEENMNLKYLLLFVYFVQVLNCCYGHGDPLSYELDRGPKCPFNSDTVS
IIIETRENRNRDLYTLQTLQNHPEFKKKTITRPVVFITHGFTSSASET
NFINLAKALVDKDNYMVISIDWQTAACTNEAAGLKYLYYPTAARNTRL
VGQYIATITQKLVKHYKISMANIRLIGHSLGAHASGFAGKKVQELKLG
KYSEIIGLDPARPSFDSNHCSERLCETDAEYVQIIHTSNYLGTEKTLG
TVDFYMNNGKNQPGCGRFFSEVCSHSRAVIYMAECIKHECCLIGIPKS
KSSQPISSCTKQECVCVGLNAKKYPSRGSFYVPVESTAPFCNNKGKII
1346323 ALLERGEN VES V 2
SERPKRVFNIYWNVPTFMCHQYDLYFDEVTNFNIKRNSKDDFQGDKIA
IFYDPGEFPALLSLKDGKYKKRNGGVPQEGNITIHLQKFIENLDKIYP
NRNFSGIGVIDFERWRPIFRQNWGNMKIHKNFSIDLVRNEHPTWNKKM
IELEASKRFEKYARFFMEETLKLAKKTRKQADWGYYGYPYCFNMSPNN
LVPECDVTAMHENDKMSWLFNNQNVLLPSVYVRQELTPDQRIGLVQGR
VKEAVRISNNLKHSPKVLSYWWYVYQDETNTFLTETDVKKTFQEIVIN
GGDGIIIWGSSSDVNSLSKCKRLQDYLLTVLGPIAINVTEAVN
549194 ALLERGEN VES V I
5KVNYCKIKCLKGGVHTACKYGTSTKPNCGKMVVKAYGLTEAEKQEIL
KVHNDFRQKVAKGLETRGNPGPQPPAKNMNNLVWNDELANIAQVWASQ
CNYGHDTCKDTEKYPVGQNIAKRSTTAALFDSPGKLVKMWENEVKDFN
PNIEWSKNNLKKTGHYTQMVWAKTKEIGCGSVKYVKDEWYTHYLVCNY
GPSGNFRNEKLYEKK

Additional vespula sequences (NCBI entrez accession):

549193; 549192; 549191; 549190; 549189; 117414; 126761; 69576; 625255; 627189; 627188; 627187; 482382; 112561; 627186; 627185; 1923233; 897645; 897647; 745570; 225764; 162551.

Tree allergen sequences (mainly birch)
sequences:
114922 Bet v 1
MGVFNYETETTSVIPAARLFKAFILDGDNLFPKVAPQAISSVENIEG
NGGPGTIKKISFPEGFPFKYVKDRVDEVDHTNFKYNYSVIEGGPIGD
TLEKISNEIKIVATPDGGSILKISNKYHTKGDHEVKAEQVKASKEMG
ETLLRAVESYLLAHSDAYN
130975 Bet v 2
MSWQTYVDEHLMCDIDGQASNSLASAIVGHDGSVWAQSSSFPQFKPQ
EITGIMKDFEEPGHLAPTGLHLGGIKYMVIQGEAGAVIRGKKGSGGI
TIKKTGQALVFGIYEEPVTPGQCNMVVERLGDYLIDQGL
1168696 Bet v 3
MPCSTEAMEKAGHGHASTPRKRSLSNSSFRLRSESLNTLRLRRIFDL
FDKNSDGIITVDELSRALNLLGLETDLSELESTVKSFTREGNIGLQF
EDFISLHQSLNDSYFAYGGEDEDDNEEDMRKSILSQEEADSFGGFKV
FDEDGDGYISARELQMVLGKLGFSEGSEIDRVEKMIVSVDSNRDGRV
DFFEFKDMMRSVLVRSS
809536 Bet v 4
MADDHPQDKAERERIFKRFDANGDGKISAAELGEALKTLGSITPDEV
KHMMAEIDTDGDGFISFQEFTDFGRANRGLLKDVAKIF
543675 Que a I-Quercus alba = oak trees
(fragment)
GVFTXESQETSVIAPAXLFKALFL
543509 Car b I-Carpinus betulus = hornbeam
trees (fragment)
GVFNYEAETPSVIPAARLFKSYVLDGDKLIPKVAPQAIXK
543491 Aln g I-Alnus glutinosa = alder trees
(fragment)
GVFNYEAETPSVIPAARLFKAFILDGDKLLPKVAPEAVSSVENI
1204056 Rubisco
VQCMQVWPPLGLKKFETLSYLPPLSSEQLAKEVDYLLRKNLIPCLEF
ELEHGFVYREHNRSPGYYDGRYWTMWKLPMFGCNDSSQVLKELEECK
KAYPSAFIRIIGFDDK

Additional tree allergen sequences (NCBI entrez accession number):

131919; 128193; 585564; 1942360; 2554672; 2392209; 2414158; 1321728; 1321726; 1321724; 1321722; 1321720; 1321718; 1321716; 1321714; 1321712; 3015520; 2935416; 464576; 1705843; 1168701; 1168710; 1168709; 1168708; 1168707; 1168706; 1168705; 1168704; 1168703; 1168702; 1842188; 2564228; 2564226; 2564224; 2564222; 2564220; 2051993; 1813891; 1536889; 534910; 534900; 534898; 1340000; 1339998; 2149808; 66207; 2129477; 1076249; 1076247; 629480; 481805; 81443; 1361968; 1361967; 1361966; 1361965; 1361964; 1361963; 1361962; 1361961; 1361960; 1361959; 320546; 629483; 629482; 629481; 541804; 320545; 81444; 541814:; 629484; 474911; 452742; 1834387; 298737; 298736; 1584322; 1584321; 584320; 1542873; 1542871; 1542869; 1542867; 1542865; 1542863; 1542861; 1542859; 1542857; 1483232; 1483230; 1483228; 558561; 551640; 488605; 452746; 452744; 452740; 452738; 452736; 452734; 452732; 452730; 452728; 450885; 17938; 17927; 17925; 17921; 297538; 510951; 289331; 289329; 166953.

Peanut
Peanut sequences
1168391 Ara h 1
MRGRVSPLMLLLGILVLASVSATHAKSSPYQKKTENPCAQRCLQSC
QQEPDDLKQKACESRCTKLEYDPRCVYDPRGHTGTTNQRSPPGERT
RGRQPGDYDDDRRQPRREEGGRWGPAGPREREREEDWRQPREDWRR
PSHQQPRKIRPEGREGEQEWGTPGSHVREETSRNNPFYFPSRRFST
RYGNQNGRIRVLQRFDQRSRQFQNLQNHRIVQIEAKPNTLVLPKHA
DADNILVIQQGQATVTVANGNNRKSFNLDEGHALRIPSGFISYILN
RHDNQNLRVAKISMPVNTPGQFEDFFPASSRDQSSYLQGFSRNTLE
AAFNAEFNEIRRVLLEENAGGEQEERGQRRWSTRSSENNEGVIVKV
SKEHVEELTKHAKSVSKKGSEEEGDITNPINLREGEPDLSNNFGKL
FEVKPDKKNPQLQDLDMMLTCVEIKEGALMLPHFNSKAMVIVVVNK
GTGNLELVAVRKEQQQRGRREEEEDEDEEEEGSNREVRRYTARLKE
GDVFIMPAAHPVAINASSELHLLGFGINAENNHRIFLAGDKDNVID
QIEKQAKDLAFPGSGEQVEKLIKNQKESHFVSARPQSQSQSPSSPE
KESPEKEDQEEENQGGKGPLLSILKAFN

Ragweed

Ambrosia Sequences

113478 Amb a 1
MGIKHICCYILYFTLALVTLLQPVRSAEDLQQILPSANETRSLTTCGT
YNIIDGCWRGKADWAENRKALADCAQGFAKGTIGGKDGDIYTVTS
ELDDDVANPKEGTLRFGAAQNRPLWIIFARDMVIRLDRELAINNDK
TIDGRGAKVEHNAGFAIYNVKNIIIHNIIMHDIVVNPGGLIKSHDGPP
VPRXGSDGDAIGISGGSQIWIDHCSLSKAVDGLIDAKHGSTHFTVSN
CLFTQHQYLLLFWDFDERGMLCTVAFNKFTDNVDQRMPNLRHGF
VQVVNNNYERWGSYALGGSAGPTILSQGNRFLASDIKKEVVGRYG
ESAMSESINWNWRSYMDVFENGAIFVPSGVDPVLTPEQNAGMIPAE
PGEAVLRLTSSAGVLSCQPGAPC
113479 Amb a 2
MGIKHCCYILYFTLALVTLVQAGRLGEEVDILPSPNDTRRSLQGCE
AHNIIDKCWRCKPDWAENRQALGNCAQGFGKATHGGKWGDIYM
VTSDQDDDVVNPKEGTLRFGATQDRPLWIIFQRDMIIYLQQEMVVT
SDKTIDGRGAKVELVYGGITLMNVKNVIIHNIDIHDVRVLPGGRIKS
NGGPAIPRHQSDGDAIHVTGSSDIWIDHCTLSKSFDGLVDVNWGST
GVTISNCKFTHHEKAVLLGASDTHFQDLKMHVTLAYNIFTNTVHE
RMPRCRFGFFQIVNNFYDRWDKYAIGGSSNPTILSQGNKFVAPDFIY
KKNVCLRTGAQEPEWMTWNWRTQNDVLENGAIFVASGSDPVLTA
EQNAGMMQAEPGDMVPQLTMNAGVLTCSPGAPC
113477 Amb a 1.3
MGIKQCCYILYFTLALVALLQPVRSAEGVGEILPSVNETRSLQACEA
LNIIDKCWRGKADWENNRQALADCAQGFAKGTYGGKWGDVYTV
TSNLDDDVANPKEGTLRFAAAQNRPLWIIFKNDMVINLNQELVVN
SDKTIDGRGVKVEIINGGLTLMNVKNIIIHNINIHDVKVLPGGMIKSN
DGPPILRQASDGDTINVAGSSQIWIDHCSLSKSFDGLVDVTLGSTHV
TISNCKFTQQSKAILLGADDTHVQDKGMLATVAFNMFTDNVDQR
MPRCRFGFFQVVNNNYDRWGTYAIGGSSAPTILCQGNRFLAPDDQI
KKNVLARTGTGAAESMAWNWRSDKDLLENGAIFVTSGSDPVLTPV
QSAGMIPAEPGEAAIKLTSSAGVFSCHPGAPC
113476 Amb a 1.2
MGIKHCCYILYFTLALVTLLQPVRSAEDVEEFLPSANETRRSLKACE
AHNIIDKCWRCKADWANNRQALADCAQGFAKGTYGGKHGDVYT
VISDKDDDVANPKEGTLRFAAAQNRPLWIIFKRNMVIHLNQELVV
NSDKTIDGRGVKVNIVNAGLTLMNVKNIIIHNINIHDIKVCPGGMIKS
NDGPPILRQQSDGDAINVAGSSQIWIDHCSLSKASDGLLDITLGSSHV
TVSNCKFTQHQFVLLLGADDTHYQDKGMLATVAFNMFTDHVDQR
MPRCRFGFFQVVNNNYDRWGTYAIGGSSAPTILSQGNRFFAPDDIIK
KNVLARTGTGNAESMSWNWRTDRDLLENGATFLPSGSDPVLTPEQ
KAGMIPAEPGEAVLRLTSSAGVLSCHQGAPC
113475 Amb a 1.1
MGIKHCCYILYFTLALVTLLQPVRSAEDLQEILPVNETRRLTTSGAY
NIIDGCWRGKADWAENRKALADCAQGFGKGTVGGKDGDIYTVTS
ELDDDVANPKEGTLRFGAAQNRPLWIIFERDMVIRLDKEMVVNSD
KTIDGRGAKVEIINAGFTLNGVKNVIIHNINMHDVKVNPGGLIKSND
GPAAPRAGSDGDAISISGSSQIWIDHCSLSKSVDGLVDAKLGTTRLT
VSNSLFTQHQFVLLFGAGDENIEDRGMLATVAFNTFTDNVDQRMP
RCRHGFFQVVNNNYDKWGSYAIGGSASPTILSQGNRFCAPDERSKK
NVLGRHGEAAAESMKWNWRTNKDVLENGAIFVASGVDPVLTPEQ
SAGMIPAEPGESALSLTSSAGVLSCQPGAPC

Cedar Sequences

493634 Cry j IB precursor
MDSPCLVALLVFSFVIGSCFSDNPIDSCWRGDSNWAQNRMKLADC
AVGFGSSTMGGKGGDLYTVTNSDDDPVNPPGLRYGATRDRPLWI
IFSGNMNIKLKMPMYIAGYKTFDGRGAQVYIGNGGPCVFIKRVSNV
IIHGLYLYGCSTSVLGNVLINESFGVEPVHPQDGDALTLRTATNIWI
DHNSFSNSSDGLVDVTLTSTGVTISNNLFFNHHKVMSLGHDDAYSD
DKSMKVTVAFNQFGPNCGQRMPRARYGLVHVANNNYDPWTIYAI
GGSSNPTILSEGNSFTAPNESYKKQVTIRIGCKTSSSCSNWVWQSTQ
DVFYNGAYFVSSGKYEGGNIYTKKEAFNVENGNATPHLTQNAGVL
TCSLSKRC
493632 Cry j IA precursor
MDSPCLVALLVLSFVIGSCFSDNPIDSCWRGDSNWAQNRMKLADC
AVGFGSSTMGGKGGDLYTVTNSDDDPVNPAPGTLRYGATRDRPL
WIIFSGNMNIKLKMPMYIAGYKTFDGRGAQVYIGNGGPCVFIKRVS
NVIIHGLHLYGCSTSVLGNVLINESFGVEPVHPQDGDALTLRTATNI
WIDHNSFSNSSDGLVDVTLSSTGVTISNNLFFNHHKVMLLGHDDAY
SDDKSMKVTVAFNQFGPNCGQRMPRARYGLVHVANNNYDPWTIY
AIGGSSNPTILSEGNSFTAPNESYKKQVTIRIGCKTSSSCSNWVWQST
QDVFYNGAYFVSSGKYEGGNIYTKKEAFNVENGNATPQLTKNAGV
LTCSLSKRC
1076242 Cry j II precursor-Japanese cedar
MAMKLIAPMAFLAMQLIIMAAAEDQSAQIMLDSVVEKYLRSNRSL
RKVEHSRHDAINIFNVEKYGAVGDGKHDCTEAFSTAWQAACKNPS
AMLLVPGSKKFVVNNLFFNGPCQPHFTFKVDGIIAAYQNPASWKN
NRIWLQFAKLTGFTLMGKGVIDGQGKQWWAGQCKWVNGREICND
RDRPTAIKFDFSTGLIIQGLKLMNSPEFHLVFGNCEGVKIIGISTAPR
DSPNTDGIDIFASKNFHLQKNTIGTGDDCVAIGTGSSNIVIEDLICGP
GHGISIGSLGRENSRAEVSYVHVNGAKYIDTQNGLPIKTWQGGSGM
ASHIIYENVEMINSENPILINQFYCTSASACQNQRSAVQIQDVTYKNI
RGTSATAAAIQLKCSDSMPCKDIKLSDISLKLTSGKIASCLNDNANG
YFSGHVIPACKNLSPSAKRKESKSHKHPKTVMVENMRAYDKGNRT
RILLGSRPPNCTNKCHGCSPCKAKLVIVHRIMPQEYYPQRWICSCHG
KIYHP
1076241 Cry j II protein-Japanese cedar
MAMKFIAPMAFVAMQLIIMAAAEDQSAQIMLDSDIEQYLRSNRSLR
KVEHSRHDAINIFNVEKYGAVGDGKHDCTEAFSTAWQAACKKPSA
MLLVPGNKKFVVNNLFFNGPCQPHFTFKVDGIIAAYQNPASWKNN
RIWLQFAKLTGFTLMGKGVIDGQGKQWWAGQCKWVNGREICNDR
DRPTAIKPDFSTGLIIQGLKLMNSPEFHLVFGNCEGVKIIGISITAPRD
SPNTDGIDIFASKNFHLQKNTIGTGDDCVAIGTGSSMVIEDLICGPG
HGISIGSLGRENSRAEVSYVHVNGAKFIDTQNGLRIKTWQGGSGMA
SHIIYENVEMINSENPILINQFYCTSASACQNQRSAVQIQDVTYKNIR
GTSATAAAIQLKCSDSMPCKDIKLSDISLKLTSGKIASCLNDNANGY
FSGHVIPACKNLSPSAKRKESKSHKHPKTVMVKNMGAYDKGNRTRI
LLGSRPPNCTNKCHGCSPCKAKLVIVHRIMPQEYYPQRWMCSRHG
KIYHP
541803 Cry j I precursor-Japanese cedar
MDSPCLVALLVLSFVIGSCFSDNPIDSCWRGDSNWAQNRMKLADC
AVGFGSSTMGGKGGDLYTVTNSDDDPVNPPGTLRYGATRDRPLWI
IFSGNMNIKLKMPMYIAGYKTFDGRGAQVYIGNGGPCVFIKRVSNV
IIHGLHLYGCSTSVLGNVLINESFGVEPVHPQDGDALTLRTATNIWI
DHNSFSNSSDGLVDVTLSSTGVTISNNLFFNHHKVMLLGHDDAYSD
DKSMKVTVAFNQFGPNCGQRMPRARYGLVHVANNNYDPWTIYAI
GGSSNPTILSEGNSFTAPNESYKKQVTIRIGCKTSSSCSNWVWQSTQ
DVFYNGAYFVSSGKYEGGNIYTKKEAFNVENGNATPQLTKNAGVL
TCSLSKRC
541802 Cry j I precursor-Japanese cedar
MDSPCLVALLVFSFVIGSCFSDNPIDSCWRGDSNWAQNRMKLADC
AVGFGSSTMGGKGGDLYTVTNSDDDPVNPAPGTLRYGATRDRPL
WIIFSGNMNIKLKMPMYIAGYKTFDGRGAQVYIGNGGPCVFIKRVS
NVIIHGLYLYGCSTSVLGNVLINESFGVEPVHPQDGDALTLRTATNI
WIDHNSFSNSSDGLVDVTLTSTGVTISNNLFFNHHKVMSLGHDDAY
SDDKSMKVTVAFNQFGPNCGQRMPRARYGLVHVANNNYDPWTIY
AIGGSSNPTILSEGNSFTAPNESYKKQVTIRIGCKTSSSCSNWVWQST
QDVFYNGAYFVSSGKYEGGNIYTKKEAFNVENGNATPHLTQNAGV
LTCSLSKRC

Dog

Canis Sequences:

Can f 1
MKTLLLTIGFSLIATLQAQDTPALGKDTVAVSGKWYLKAMTADQE
VPEKPDSVTPMILKAQKGGNLEAKITMLTNGQCQNITVVLHKTSEP
GKYTAYEGQRVVFIQPSPVRDHYILYCEGELHGRQIRMAKLLGRDP
EQSQEALEDFREFSRAKGLNQEILELAQSETCSPGGQ
Serum albumin fragment
EAYKSEIAHRYNDLGEEHFRGLVL
Serum albumin fragment
LSSAKERFKCASLQKFGDRAFKAWSVARLSQRFPKADFAEISKVVT
DLTKVHKECCHGDLLECADDRADLAKYMCENQDSISTKLKECCDK
PVLEKSQCLAEVERDELPGDLPSLAADFVEDKEVCKNYQEAKDVF
LGTFLYEYSRRHPEYSVSLLLRIAKEYEATLEKCCATDDPPTCYAK
VLDEFKPLVDEPQNLVKTNCELFEKLGEYGFQNALLVRYTKKAPQ
VSTPTLVVEVSRKLGKVGTKCCKKPESERMSCADDFLS
Can f 2
MQLLLLTVGLALICGLQAQEGNHEEPQGGLEELSGRWHSVALASN
KSDLIKPWGHFRVFIHSMSAKDGNLHGDILIPQDGQCEKVSLTAFKT
ATSNKFDLEYWGHNDLYLAEVDPKSYLILYMINQYNDDTSLVAHL
MVRDLSRQQDFLPAFESVCEDIGLHKDQIVVLSDDDRCQGSRD

Additional dog allergen protein (NCBI entrez accession):

1731859

Horse

Equus Sequences:

1575778 Equ c1
MKLLLLCLGLILVCAQQEENSDVAIRNFDISKISGEWYSIFLASDVK
EKIEENGSMRVFVDVIRALDNSSLYAEYQTKVNGECTEFPMVFDKT
EEDGVYSLNYDGYNVFRISEFENDEHIILYLVNFDKDRPFQLFEFYA
REPDVSPEIKEEFVKIVQKRGIVKENIIDLTKIDRCFQLRGNGVAQA
3121755 Equ c 2
SQXPQSETDYSQLSGEWNTIYGAASNIXK
Euroglyphus (mite)
Euroglyphus sequences:
Eur m 1 (variant)
TYACSINSVSLPSELDLRSLRTVTPIRMQGGCGSCWAFSGVASTESA
YLAYRNMSLDLAEQELVDCASQNGCHGDTIPRGIEYIQQNGVVQE
HYYPYVAREQSCHRPNAQRYGLKNYCQISPPDSNKIRQALTQTHTA
VAVIIGIKDLNAFRHYDGRTIMQHDNGYQPNYHAVNIVGYGNTQG
VDYWIVRNSWDTTWGDNGYGYFAANINL
Eur m 1 (variant)
TYACSINSVSLPSELDLRSLRTVTPIRMQGGCGSCWAFSGVASTESA
YLAYRNMSLDLAEQELVDCASQNGCHGDTIPRGIEYIQQNGVVQE
HYYPYVAREQSCHRPNAQRYGLKNYCQISPPDSNKIRQALTQTHTA
VAVIIGIKDLNAFRHYDGRTIMQHDNGYQPNYHAVNIVGYGNTQG
VDYWIVRNSWDTTWGDNGYGYFAANINL
Eur m 1 (variant)
ETNACSINGNAPAEIDLRQMRTVTPIRMQGGCGSCWAFSGVAATES
AYLAYRNQSLDLAEQELVDCASQHGCHGDTIPRGIEYIQHNGVVQE
SYYRYVAREQSCRRPNAQRFGISNYCQIYPPNANKIREALAQTHSAI
AVHGTKDLDAFRHYDGRTIIQRDNGYQPNYHAVNIVGYSNAQGVD
YWIVRNSWDTNWGDNGYGYFAANIDL
Eur m 1 (variant)
ETSACRINSVNVPSELDLRSLRTVTPIRMQGGCGSCWAFSGVAATES
AYLAYRNTSLDLSEQELVDCASQHGCHGDTIPRGIEYIQQNGVVEE
RSYPYVAREQQCRRPNSQHYGISNYCQIYPPDVKQIREALTQTHTAI
AVIIGIKDLRAFQHYDGRTIIQHDNGYQPNYHAVNIVGYGSTQGVD
YWIVRNSWDTTWGDSGYGYFQAGNNL
Poa (grass) sequences
113562 POLLEN ALLERGEN POA P 9
MAVQKYTVALFLVALVVGPAASYAADLSYGAPATPAAPAAGYTP
AAPAGAAPKATTDEQKMIEKINVGFKAAVAAAGGVPAANKYKTFV
ATFGAASNKAFAEALSTEPKGAAVDSSKAALTSKLDDAAYKLAYKS
AEGATPEAKYDDYVATLSEALRIIAGTLEVHGVKPAAEEVKATPAG
ELQVIDKVDAAFKVAATAANAAPANDKFTVFEAAFNDAIKASGG
AYQSYKFIPALEAAVKQSYAATVATAPAVKYTVFETALKKAITAMS
QAQKAAKPAAAATGTATAAVGAATGAATAAAGGYKV
113561 POA P 9
MAVHQYTVALFLAVALVAGPAASYAADVGYGAPATLATPATPAA
PAAGYTPAAPAGAAPKATTDEQKLIEKINAGFKAAVAAAAGVPAV
DKYKTFVATFGTASNKAFAEALSTEPKGAAAASSNAVLTSKLDAA
YKLAYKSAEGATPEAKYDAYVATLSEALRIIAGTLEVHAVKPAGEE
VKAIPAGELQVIDKVDAAFKVAATAANAAPANDKYTVFEAAFNDA
IKASTGGAYQSYKFIPALEAAVKQSYAATVATAPAVKYTVFETALK
KAITAMSQAQKAAKPAAAVTATATGAVGAATGAVGAATGAATAA
AGGYKTGAATPTAGGYKV
113560 POA P 9
MDKANGAYKTALKAASAVAPAEKFPVFQATFDKNLKEGLSGPDA
VGFAKKLDAFIQTSYLSTKAAEPKEKFDLFVLSLTEVLRFMAGAVK
APPASKFPAKPAPKVAAYTPAAPAGAAPKATTDEQKLIEKINVGFK
AAVAAAAGVPAASKYKTFVATFGAASNKAFAEALSTEPKGAAVAS
SKAVLTSKLDAAYKLAYKSAEGATPEAKYDAYVATLSEALRIIAGT
LEVHGVKPAAEEVKAIPAGELQVIDKVDAAFKVAATAANAAPAND
KFTVFEAAFNDAIKASTGGAYQSYKFIPALEAAVKQSYAATVATAP
AVKYTVFETALKKAITAMSQAQKAAKPAAAVTGTATSAVGAATGA
ATAAAGGYKV

Cockroach Sequences

2833325 Cr p1
MKTALVFAAVVAFVAARFPDHKDYKQLADKQFLAKQRDVLRLFH
RVHQHNILNDQVEVGIPMTSKQTSATTVPPSGEAVHGVLQEGHARP
RGEPFSVNYEKHREQAIMLYDLLYFANDYDTFYKTACWARDRVN
EGMFMYSFSIAVFHRDDMQGVMLPPPYEVYPYLFVDHDVIHMAQ
KYWMKNAGSGEHHSHVIPVNFTLRTQDHLLAYFTSDVNLNAFNTY
YRYYYPSWYNTTLYGHNIDRRGEQFYYTYKQIYARYFLERLSNDLP
DVYPFYYSKPVKSAYNPNLRYHNGEEMPVRPSNMYVTNFDLYYIA
DIKNYEKRVEDAIDFGYAPDEHMKPHSLYHDVHGMEYLADMIEG
NMDSPNFYFYGSIYHMYHSMIGHIVDPYHKMGLAPSLEHPETVLR
DPVFYQLWKRVDHLFQKYKNRLPRYTHDELAFEGVKVENVDVGK
LYTYFEQYDMSLDMAVYVNNVDQISNVDVQLAVRLNHKPFTYNIE
VSSDKAQDVYVAVFLGPKYDYLGREYDLNDRRHYFVEMDRFPYH
VGAGKTVIERNSHDSNIIAPERDSYRTFYKKVQEAYEGKSQYYVDK
GHNYCGYPENLLIPKGKKGGQAYTFYVIVTPYVKQDEHDFEPYNY
KAFSYCGVGSERKYPDNKPLGYPFDRKIYSNDFYTPNMYFKDVIIF
HKKYDEVGVQGH
2231297 Cr p2
INEIHSIIGLPPFVPPSRRHARRGVGINGLIDDVIAILPVDELKALFQE
KLETSPDFKALYDAIRSPEFQSIISTLNAMQRSEHHQNLRDKGVDVD
HFIQLIRALFGLSRAARNLQDDLNDFLHSLEPISPRHRHGLPRQRRR
SARVSAYLHADDFHKIITTIEALPEFANFYNFLKEHGLDVVDYINEI
HSIIGLPPFVPPSRARRGVGINGLIDDVIAILPVDELKALFQEKLET
SPDFKALYDAIRSPEFQSIISTLNAMPEYQELLQNLRDKGVDVDHFI
RVDQGTLRTLSSGQRNLQDDLNDFLALIPTDQILAIAMDYLANDAE
VQELVAYLQSDDFHKAITTIEALPEFANFYNFLKEHGLDVVDYINEI
HSIIGLPPFVPPSQRHARRGVGINGLIDDVIAILPVDELKALFQEKLET
SPDFKALYDAIDLRSSRA
1703445 Bla g 2
MIGLKLVTVLFAVATITHAAELQRVPLYKLVHVFINTQYAGITMGN
QNFLTVFDSTSCNVVVASQECVGGACVCPNLQKYEKLKPKYISDG
NVQVKFFDTGSAVGRGIEDSLTISNLTTSQQDIVLADELSQEVCILSA
DVVVGIAAPGCPNALKGKTVLENFVEENLIAPVFSIHHARFQDGEH
FGEIIFGGSDWKYVDGEFTYVPLVGDDSWKFRLDGVKIGDTTVAPA
GTQAHDTSKAIIVGPKAYVNPINEAIGCVVEKTTTRRICKLDCSKIPS
LPDVTFVINGRNFNISSQYYIQQNGNLCYSGFQPCPHSDHFFIGDFF
VDHYYSEFNWENKTMGFGRSVE
SV
1705483 Bla g 4
AVLALCATDTLANEDCFRHESLVPNLDYERFRGSWIIAAGTSEALT
QYKCWIDRFSYDDALVSKYTDSQGKNRTTIRGRTKFEGNKFTIDYN
DKGKAFSAPYSVLATDYENYAIVEGCPAAANCHVIYVQIRFSVRRF
HPKLGDKEMIQHYTLDQVNQHKKAIEEDLKHFNLKYEDLHSTCH
2326190 Bla g 5
YKLTYCPVKALGEPIRFLLSYGEKDFEDYRFQEGDWPNLKPSMPFG
KTPVLEIDGKQTHQSVAISRYLGKQFGLSGKDDWENLEIDMIVDTIS
DFRAAIANYHYDADENSKQKKWDPLKKETIPYYTKKFDEVVKANG
GYLAAGKLTWADFYFVAILDYLNHMAKEDLVANQPNLKALREKV
LGLPAIKAWVAKRPPTDL

Additional cockroach sequences (NCBI Entrez accession numbers):

2580504; 1580797; 1580794; 1362590; 544619; 544618; 1531589; 1580792; 1166573; 1176397; 2897849.

Allergen (General) Sequences:

NCBI Accession Numbers

2739154; 3719257; 3703107; 3687326; 3643813; 3087805; 1864024; 1493836; 1480457; 2598976; 2598974; 1575778; 763532; 746485; 163827; 163823; 3080761; 163825; 3608493; 3581965; 2253610; 2231297; 2897849; 3409499; 3409498; 3409497; 3409496; 3409495; 3409494; 3409493; 3409492; 3409491; 3409490; 3409489; 3409488; 3409487; 3409486; 3409485; 3409484; 3409483; 3409482; 3409481; 3409480; 3409479; 3409478; 3409477; 3409476; 3409475; 3409474; 3409473; 3409472; 3409471; 3409470; 3409469; 3409468; 3409467; 3409466; 3409465; 3409464; 3409463; 3409462; 3409461; 3409460; 3409459; 3409458; 3409457; 3409456; 3318885; 3396070; 3367732; 1916805; 3337403; 2851457; 2851456; 1351295; 549187; 136467; 1173367; 2499810; 2498582; 2498581; 1346478; 1171009; 126608; 114091; 2506771; 1706660; 1169665; 1169531; 232086; 416898; 114922; 2497701; 1703232; 1703233; 1703233; 1703232; 3287877; 3122132; 3182907; 3121758; 3121756; 3121755; 3121746; 3121745; 3319925; 3319923; 3319921; 3319651; 3318789; 3318779; 3309647; 3309047; 3309045; 3309043; 3309041; 3309039; 3288200; 3288068; 2924494; 3256212; 3256210; 3243234; 3210053; 3210052; 3210051; 3210050; 3210049; 3210048; 3210047; 3210046; 3210045; 3210044; 3210043; 3210042; 3210041; 3210040; 3210039; 3210038; 3210037; 3210036; 3210035; 3210034; 3210033; 3210032; 3210031; 3210030; 3210029; 3210028; 3210027; 3210026; 3210025; 3210024; 3210023; 3210022; 3210021; 3210020; 3210019; 3210018; 3210017; 3210016; 3210015; 3210014; 3210013; 3210012; 3210011; 3210010; 3210009; 3210008; 3210007; 3210006; 3210005; 3210004; 3210003; 3210002; 3210001; 3210000; 3209999; 3201547; 2781152; 2392605; 2392604; 2781014; 1942360; 2554672; 2392209; 3114481; 3114480; 2981657; 3183706; 3152922; 3135503 3135501; 3135499; 3135497; 2414158; 1321733; 1321731; 1321728; 1321726; 1321724; 1321722; 1321720; 1321718; 1321716; 1321714; 1321712; 3095075; 3062795; 3062793; 3062791; 2266625; 2266623; 2182106; 3044216; 2154736; 3021324; 3004467; 3005841; 3005839; 3004485; 3004473; 3004471; 3004469; 3004465; 2440053; 1805730; 2970629; 2959898; 2935527; 2935416; 809536; 730091; 585279; 584968; 2498195; 2833325; 2498604; 2498317; 2498299; 2493414; 2498586; 2498585; 2498576; 2497749; 2493446; 2493445; 1513216; 729944; 2498099; 548449; 465054; 465053; 465052; 548671; 548670; 548660; 548658; 548657; 2832430; 232084; 2500822; 2498118; 2498119; 2498119; 2498118; 1708296; 1708793; 416607; 416608; 416608; 416607; 2499791; 2498580; 2498579; 2498578; 2498577; 2497750; 1705483; 1703445; 1709542; 1709545; 1710589; 1352699; 1346568; 1346323; 1346322; 2507248; |1352240; 1352239; 1352237; 1352229; 1351935; 1350779; 1346806; 1346804; 1346803; 1170095; 1168701; 1352506; 1171011; 1171008; 1171005; 1171004; 1171002; 1171001; 1168710; 1168709; 1168708; 1168707; 1168706; 1168705; 1168704; 1168703; 1168702; 1168696; 1168391; 1168390; 1168348; 1173075; 1173074; 1173071; 1169290; 1168970; 1168402; 729764; 729320; 729979; 729970; 729315; 730050; 730049; 730048; 549194; 549193; 549192; 549191; 549190; 549189; 549188; 549185; 549184; 549183; 549182; 549181; 549180; 549179; 464471; 585290; 416731; 1169666; 113478; 113479; 113477; 113476; 113475; 130975; 119656; 113562; 113561; 113560; 416610; 126387; 126386; 126385; 132270; 416611; 416612; 416612; 416611; 730035; 127205; 1352238; 125887; 549186; 137395; 730036; 133174; 114090; 131112; 126949; 129293; 124757; 129501; 416636; 2801531; 2796177; 2796175; 2677826; 2735118; 2735116; 2735114; 2735112; 2735110; 2735108; 2735106; 2735104; 2735102; 2735100; 2735098; 2735096; 2707295; 2154730; 2154728; 1684720; 2580504; 2465137; 2465135; 2465133; 2465131; 2465129; 2465127; 2564228; 2564226; 2564224; 2564222; 2564220; 2051993; 1313972; 1313970; 1313968; 1313966; 2443824; 2488684; 2488683; 2488682; 2488681; 2488680; 2488679; 2488678; 2326190; 2464905; 2415702; 2415700; 2415698; 2398759; 2398757; 2353266; 2338288; 1167836; 414703; 2276458; 1684718 2293571; 1580797; 1580794; 2245508; 2245060; 1261972; 2190552; 1881574 511953; 1532058; 1532056; 1532054; 1359436; 666007; 487661; 217308; 1731859; 217306; 217304; 1545803; 1514943; 577696; 516728; 506858; 493634; 493632; 2154734; 2154732; 543659; 1086046: 1086045; 2147643; 2147642; 1086003; 1086002; 1086001; 543675; 543623; 543509; 543491; 1364099; 2147108; 2147107; 1364001; 1085628; 631913; 631912; 631911; 2147092; 477301; 543482; 345521; 542131; 542130; 542129; 100636; 2146809; 480443; 2114497; 2144915; 72355; 71728; 319828; 1082946; 1082945; 1082944; 539716; 539715; 423193; 423192; 423191; 423190; 1079187; 627190; 627189; 627188; 627187; 482382; 1362656; 627186; 627185; 627182; 482381; 85299; 85298; 2133756; 2133755; 1079186; 627181; 321044; 321043; 112559; 112558; 1362590; 2133564; 1085122; 1078971; 627144; 627143; 627142; 627141; 280576; 102835; 102834; 102833; 102832; 84703; 84702; 84700; 84699; 84698; 84696; 477888; 477505; 102575; 102572; 478272; 2130094; 629813; 629812; 542172; 542168; 542167; 481432; 320620; 280414; 626029; 542132; 320615; 320614; 100638; 100637; 100635; 82449; 320611; 320610; 280409; 320607; 320606; 539051; 539050; 539049; 539048; 322803; 280407; 100501; 100498; 100497; 100496; 1362137; 1362136; 1362135; 1362134; 1362133; 1362132; 1362131; 1362130; 1362129; 1362128; 100478; 2129891; 1076531; 1362049; 1076486; 2129817; 2129816; 2129815; 2129814; 2129813; 2129812; 2129805; 2129804; 2129802; 2129801; 2129800; 2129799; 479902; 479901; 2129477; 1076247; 629480; 1076242; 1076241; 541803; 541802; 280372; 280371; 1361968; 1361967; 1361966; 1361965; 1361964; 1361963; 1361962; 1361961; 1361960; 1361959; 320546; 2119763; 543622; 541804; 478825; 478824; 478823; 421788; 320545; 81444; 626037; 626028; 539056; 483123; 481398; 481397; 100733; 100732; 100639; 625532; 1083651; 322674; 322673; 81719; 81718; 2118430; 2118429; 2118428; 2118427; 419801; 419800; 419799; 419798; 282991; 100691; 322995; 322994; 101824; 626077; 414553 398830; 1311457; 1916292; 1911819; 1911818; 1911659; 1911582; 467629; 467627; 467619; 467617; 915347; 1871507; 1322185; 1322183; 897645; 897647; 1850544; 1850542; 1850540; 288917; 452742; 1842045; 1839305; 1836011; 1836010; 1829900; 1829899; 1829898; 1829897; 1829896; 1829895; 1829894; 1825459; 1808987 159653; 1773369; 1769849; 1769847; 608690; 1040877; 1040875; 1438761; 1311513; 1311512; 1311511; 1311510; 1311509; 1311689; 1246120; 1246119; 1246118; 1246117; 1246116; 1478293; 1478292; 1311642; 1174278; 1174276; 1086972; 1086974; 1086976; 1086978; 1086978; 1086976; 1086974; 1086972; 999009; 999356; 999355; 994866; 994865; 913758; 913757; 913756; 913285; 913283; 926885; 807138; 632782; 601807; 546852; 633938; 544619; 544618; 453094; 451275; 451274; 407610; 407609; 404371; 409328; 299551; 299550; 264742; 261407; 255657; 250902; 250525; 1613674; 1613673; 1613672; 1613671; 1613670; 1613304; 1613303; 1613302; 1613240; 1613239; 1613238; 1612181; 1612180; 1612179; 1612178; 1612177; 1612176; 1612175; 1612174; 1612173; 1612172; 1612171; 1612170; 1612169; 1612168; 1612167; 1612166; 1612165; 1612164; 1612163; 1612162; 1612161; 1612160; 1612159; 1612158; 1612157; 1612156; 1612155; 1612154; 1612153; 1612152; 1612151; 1612150; 1612149; 1612148; 1612147; 1612146; 1612145; 1612144; 1612143; 1612142; 1612141; 1612140; 1612139; 1093120; 447712; 447711; 447710; 1587177; 158542; 1582223; 1582222; 1531589; 1580792; 886215; 1545897; 1545895; 1545893; 1545891; 1545889; 1545887; 1545885; 1545883; 1545881; 1545879; 1545877; 1545875; 166486; 1498496; 1460058; 972513; 1009442; 1009440; 1009438; 1009436; 1009434; 7413; 1421808; 551228; 452606; 32905; 1377859; 1364213; 1364212; 395407; 22690; 22688; 22686; 22684; 488605; 17680; 1052817; 1008445; 1008443; 992612; 706811; 886683; 747852; 939932; 19003; 1247377; 1247375; 1247373; 862307; 312284; 999462; 999460; 999458; 587450; 763064; 886209; 1176397; 1173557; 902012; 997915; 997914; 997913; 997912; 997911; 997910; 99790; 997908; 997907; 997906; 997905; 997904; 997903; 997902; 997901; 997900; 997899; 997898; 997897; 997896; 997895; 997894; 997893; 997892; 910984; 910983; 910982; 910981; 511604; 169631; 169629; 169627; 168316; 168314; 607633; 555616; 293902; 485371; 455288; 166447; 166445; 166443; 166435; 162551; 160780; 552080; 156719; 156715; 515957; 515956; 515955; 515954; 515953; 459163; 166953; 386678; 169865.

### Example 7

### Desensitisation Using Multiple Overlapping Peptides (MOP) from Fel d I

We have obtained data with multiple overlapping peptides (MOP) which are derived from the sequence of Fel d I and include the three FC1P peptides. Originally, 16 peptides spanning both chain 1 and chain 2 of the Fel d I molecule were designed in order to increase the percentage of individuals reacting to the peptide injection. By using peptides covering the entire molecule, we believed that we would cover more MHC-peptide pairings and thus get more reactors. Of the 16 peptides, the first three of chain 2 displayed poor solubility in aqueous solution and were excluded from the in vivo preparation termed MOP. The sequences of the MOP peptides and how they relate to the parent molecule are given in FIG. 9.

We have carried-out a dose ranging study with this preparation to determine an appropriate dose to be used in a planned clinical trial in which four injections of increasing dose will be given over a two week period. For the dose ranging study, three doses have been tested: 1 μg (of each of the 13 peptides in a mixture), 2.5 μg and 5 μg.

Four cat asthmatic individuals received the 1 μg dose. One of them developed a LAR which was similar to those induced with FC1P. Five individuals received 2.5 μg and again one developed a LAR. At 5 μg, eight individuals were tested and four developed a LAR. This demonstrates the dose response effect that we expected and, more importantly, shows that the MOP preparation produces a similar effect to the FC1P preparation. An example of a LAR induced by MOP can be seen in FIG. 10.

Rather than move to a higher dose which may give a higher percentage of LAR reactors, we have decided to use the 5 μg dose as the starting dose for the trial. From the number of peptides in the MOP preparation and the observed dose response, it might be expected that some of the non-LAR reactors at 5 μg might develop a LAR at a higher dose, ie have the appropriate MHC molecules to recognise the peptides but experienced a “sub-clinical” reaction. For this reason, we decided to investigate the cutaneous late phase reaction to whole allergen extract as an alternative clinical outcome. Basically, if whole allergen extract is injected intradermally (in our case into the forearm) in an atopic allergic individual, an immediate wheal and flare reaction will result (classical IgE mediated early allergic reaction) in about 15 minutes. This reaction is then followed by a delayed in-time phase reaction in the skin. Like the lung reaction, this peaks at 6-9 hours and believed to be driven at least in part by T cells.

Previously, immunotherapy studies using conventional whole allergen extract have demonstrated that the size of this late phase skin reaction decreases after several months of treatment.

We have measured these skin reactions before any peptide injection (ie at baseline) and we have measured them again in six patients (to date) who have had either one or two injections only of MOP. All six have reduced reactions as shown in FIG. 11. These results are statistically significant with a p value of 0.036.

A further interesting observation was that some of these individuals did not develop a lung reaction (LAR) to the MOP injection but clearly their T cells were activated by one or more of the peptides giving them a measurable reduction in reactivity to skin challenge with whole allergen extract (the latter being perhaps even more significant since the whole dander extract contains multiple proteins (including Fel d I) to which the patient may be sensitised).

As mentioned above, some (three) of the MOP injected individuals who developed lung reactions have received a second injection. As found with FC1P, these individuals did not develop reactions (an example can be seen in FIG. 10). Importantly, these two individuals received the second injection several weeks (in one case about 4 months) after the first one. This suggests that hyporesponsiveness induced after the first injection could last four months or more.

We also have other longitudinal data regarding the length of duration of the hyporesponsiveness from some of the FC1P patients. In this case, three patients who had received FC1P more than one year ago and experienced LAR's were rechallenged with the same dose. All three reacted with almost exactly the same magnitude as the initial reaction (FIG. 12a, b & c). Of these three, one (FIG. 12a) had received a second injection of FC1P a few weeks after the first and had displayed no LAR. Thus, peptides can induce a LAR which is followed by hyporesponsiveness which seems to last for four months (possibly more) but less than one year.

Finally, we now have three FC1P patients who have had one injection followed by a LAR which on reinjection was not seen (ie hyporesponsiveness). We also have the same finding in two MOP patients. We have had the areas under these curves analysed statistically. We have compared a control day (either saline injection or injection or whole cat extract, the latter does not induce a lung reaction only a skin reaction at the dose used), with the lung measurements (FEV1) after the first FC1P or MOP injection and after the second FC1P/MOP injection.

We have compared the mean values from spirometry by area under the curve analysis:

1. Control day vs peptide day 1 (we expect to see a significant difference i.e. there has actually been a significant reaction)

2. Control day vs peptide day 2 (do not expect a significant reaction since lung responses are back to normal)

3. Peptide day 1 vs peptide day 2 (expect a significant difference).

The results (p values) are:

1. p=0.0205

2. p=0.0930

3. p=0.0119

A p value of less than or equal to 0.05 is considered statistically significant.

Thus, there is a significant response to the peptides following the first injection (1) which is significantly different to the second injection (3) as the FEVI values appear to return to baseline. The difference between the control day and the second injection is not statistically significant (2).

1. A method of desensitising a patient to a polypeptide allergen the method comprising administering to the patient a peptide derived from the allergen wherein restriction to a MHC Class II molecule possessed by the patient can be demonstrated for the peptide and the peptide is able to induce a late phase response in an individual who possesses the said MHC Class II molecule.

2. A method according to claim 1 wherein the peptide is included in a composition containing a plurality of peptides derived from the said allergen.

3. A method according to claim 2 wherein the plurality of peptides derived from said allergen includes peptides for which restriction to Class II DR molecules DR2, DR3, DR4 and DR7 can be demonstrated, provided that such peptides can be derived from the allergen.

4. A method according to claim 1 wherein the patient possesses any one of the MHC Class II DR molecules DR2, DR3, DR4 or DR7.

5. A method according to claim 1 wherein the patient possesses the MHC Class II molecule DR4:

6. A method according to claim 2 wherein the composition contains the Fel d I-derived peptides as given in SEQ ID Nos. 1, 2 and 3.

7. A method according to claim 2 wherein the composition contains the soluble MHC Class II-restricted peptides of the Fel d I-derived peptides described in FIG. 9.

8. A composition comprising a plurality of peptides derived from a polypeptide allergen wherein for at least one of the peptides in the composition restriction to a MHC Class II molecule can be demonstrated and the composition is able to induce a late phase response in an individual possessing the given MHC Class II molecule.

9. A composition according to claim 8 wherein at least one peptide is present in the composition for which restriction to each of MHC Class II DR molecules DR2, DR3, DR4 and DR7 can be demonstrated, provided that such peptides can be derived from the allergen.

10. A composition according to any one of claims 8 or 9 for use in medicine.

11. A pharmaceutical formulation comprising a composition according to any one of claims 8 or 9 and a pharmaceutically acceptable carrier.

12. A method according to claim 1 wherein a composition according to any one of claims 8 or 9 is administered to the patient.

13. A method according to claim 1, or a composition according to any one of claims 8 or 9, or a pharmaceutical preparation according to claim 11 wherein the polypeptide allergen is any one of Fel d 1, Der p I, Der p II, Der fI or Der fII and allergens present in any of the following: grass, tree and weed (including ragweed) pollens; fungi and moulds; foods, stinging insects, the chirnomidae (non-biting midges); spiders and mites, housefly, fruit fly, sheep blow fly, screw worm fly, grain weevil, silkworm, honeybee, non-biting midge larvae, bee moth larvae, mealworm, cockroach, larvae of Tenibriomolitor beetle, mammals such as cat, dog, horse, cow, pig, sheep, rabbit, rat, guinea pig, mice and gerbil.

14. A composition according to claims 8 or 9, or a pharmaceutical preparation according to claim 11 wherein the polypeptide allergen is Fel d I and as given in SEQ ID Nos. 1, 2 and 3, or the composition contains the soluble MHC Class II-restricted peptides of the Fel d I-derived peptides described in FIG. 9.

15. A method of selecting a peptide for use as an immunotherapeutic agent for desensitising a patient to a polypeptide allergen capable of eliciting an allergic response in the patient, which patient possesses a particular MHC Class II molecule, the method comprising the steps of (1) selecting a candidate peptide derived from the polypeptide allergen, (2) determining whether the candidate peptide demonstrates restriction to the said MHC Class II molecule, and (3) determining whether the candidate peptide is able to induce a late phase response in an individual who possesses the said MHC Class II molecule.

16. A method according to claim 15 wherein step (2) is carried out prior to step (3) and only candidate peptides which demonstrate restriction to the particular MHC Class II molecule are selected for testing in step (3).

17. A method according to claim 16 wherein candidate peptides capable of inducing a late phase response and which demonstrate restriction to the particular MHC Class II molecule are selected as an immunotherapeutic agent.

18. A method according to claim 15 wherein determination of whether the candidate peptide demonstrates restriction to the said MHC Class II molecule is by using a T cell proliferation assay.

19. A method according to claim 15 wherein the allergen is selected from the group as defined in claim 13.

20. A method according to claim 15 wherein in step (2) determination of whether the candidate peptide demonstrates restriction to the said MHC Class II molecule is by using the patient's cells in a T cell proliferation assay, and in step (3) determining whether the candidate peptide is able to induce a late phase response in the patient.

21. A method according to claim 15 wherein the MHC molecule is any one of HLA-DR, HLA-DP, HLA-DQ, or subclasses thereof.

22. A peptide when selected by claim 15.

23. A database of peptides characterised according to their ability to bind an MHC Class II molecule and induce a late phase response in an individual possessing the said MHC Class II molecule.

24. A peptide listed in a database according to claim 23, for use in therapy.

25. A method for selecting a peptide for use as an immunotherapeutic agent for desensitising a patient to an allergen comprising the steps of: a) tissue-typing the patient to determine MHC Class II type; and b) selecting, from a database of peptides which are known to bind to particular MHC Class II molecules and induce a late phase response in an individual possessing such MHC Class II molecules, one or more peptides capable of binding to the MHC Class II molecules possessed by the patient.

26. A method of determining an initial dose of an immunotherapeutic peptide for desensitising a patient to a polypeptide allergen, which peptide is derived from the allergen and wherein restriction to a MHC Class II molecule possessed by the patient can be demonstrated for the peptide and the peptide is able to induce a late phase response in an individual who possesses the said MHC molecule, the method comprising (1) determining the dose which is able to generate an observable late phase response in a given proportion of individuals who possess the said MHC molecule and in whom the peptide is able to induce a late phase response and (2) selecting a lower dose which is incapable of inducing an observable late phase response in substantially all individuals who possess the said MHC molecule and in whom the peptide is able to induce a late phase response.

27. A method according to claim 26 wherein in step (1) the given proportion is 50%.

28. A method according to claim 26 wherein the lower dose is 0.01% of the dose which is able to generate an observable late phase response in the given proportion of individuals.

29. A method according to claim 26 wherein in steps (1) and (2) the peptide is included in a plurality of peptides derived from the said allergen.
